(19) United States
(12) Patent Application Publication
(10) Pub. No.: US 2022/0002730 A1
(43) Pub. Date: Jan. 6, 2022

(54) OLIGONUCLEOTIDE INHIBITORS OF NUCLEAR FACTOR KAPPA-LIGHT-CHAIN-ENHANCER OF ACTIVATED B CELLS AND THE USES THEREOF

(71) Applicant: City of Hope, Duarte, CA (US)

(72) Inventors: Marcin Tomasz Kortylewski, Monrovia, CA (US); Piotr Marek Swiderski, San Dimas, CA (US)

(21) Appl. No.: 17/269,229

(22) PCT Filed: Aug. 23, 2019
(86) PCT No.: PCT/US19/47888

§ 371 Date: Feb. 17, 2021

Related U.S. Application Data
(60) Provisional application No. 62/722,728, filed on Aug. 24, 2018.

(51) Int. Cl. C12N 15/117; A61K 47/54; A61P 35/00

(52) U.S. Cl. CPC C12N 15/117; A61K 47/548; A61P 35/00; C12N 2320/30; C12N 2310/13; C12N 2310/17; C12N 2310/3519; C12N 2310/315

(57) ABSTRACT

Disclosed herein, inter alia, are oligonucleotide inhibitors of the Nuclear Factor Kappa-Light-Chain-Enhancer of Activated B Cells (NF-κB) signaling pathway and methods of use thereof.

### CROSS-REFERENCES TO RELATED APPLICATIONS

This application claims the benefit of U.S. Provisional Application No. 62/722,728, filed Aug. 24, 2018, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant nos. R01 CA213131 and P50 CA107399 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048440-710001WO_Sequence_Listing_ST25.txt, created Aug. 22, 2019, 41,482 bytes, machine format IBM-PC, MS Windows operating system, is hereby incorporated by reference.

### BACKGROUND

Despite of recent advances in treatment of non-Hodgkin B-cell lymphoma (BCL), significant number of patients develops resistance to therapy leading to cancer relapse. The Nuclear Factor Kappa-Light-Chain-Enhancer of Activated B Cells (NF-κB) signaling pathway, which plays critical role in cancer cell survival and proliferation, is known to be partly responsible for treatment resistance in BCL. Despite being an attractive molecular target, transcription factors such as NF-κB are challenging pharmacologic targets. Disclosed here, inter alia, are solutions to these and other problems in the art.

### BRIEF SUMMARY

In an aspect is provided a compound including a first nucleic acid sequence capable of binding to Nuclear Factor Kappa-Light-Chain-Enhancer of Activated B Cells (NF-κB) and a second nucleic acid sequence capable of binding a Toll-like receptor (TLR) protein covalently bound to the first nucleic acid.

In an aspect is provided a pharmaceutical composition including a pharmaceutically acceptable excipient and a compound, or pharmaceutically acceptable salt thereof, described herein (including in an aspect, embodiment, table, figure, claim, sequence listing, or example).

In an aspect is provided a method of treating cancer in a patient in need of such treatment, the method including administering a therapeutically effective amount of a compound as described herein, including in an aspect, embodiment, table, figure, claim, sequence listing, or example, to the patient.

In an aspect is provided a method of treating graft-versus-host disease in a patient in need of such treatment, the method including administering a therapeutically effective amount of a compound as described herein, including in an aspect, embodiment, table, figure, claim, sequence listing, or example, to the patient.

In an aspect is provided a method of treating an autoimmune disease in a patient in need of such treatment, the method including administering a therapeutically effective amount of a compound as described herein, including in an aspect, embodiment, table, figure, claim, sequence listing, or example, to the patient.

In an aspect is provided a method of treating an inflammatory disease in a patient in need of such treatment, the method including administering a therapeutically effective amount of a compound as described herein, including in an aspect, embodiment, table, figure, claim, sequence listing, or example, to the patient.

In an aspect is provided a method of treating an infectious disease in a patient in need of such treatment, the method including administering a therapeutically effective amount of a compound as described herein, including in an aspect, embodiment, table, figure, claim, sequence listing, or example, to the patient.

### BRIEF DESCRIPTION OF THE DRAWINGS

FIGS. 1A-1F. Design of B-cell specific CpG-NFκBdODN (or Bc-NFκBdODN) and its cell-selective internalization into B-cell lymphoma (BCL) and various immune cells. FIG. 1A: The design of Bc-NFκBdODN conjugate with the NF-κB consensus DNA binding sequence marked by a box. 5′ T*C*C*A*T*G*A*C*G*T*T*C*C*T*G*A*T*G*C*T-ooooo-C*C*T*TGA AGG GAT TTC CCT CC-oooo-GG AGG GAA ATC CCT TCA*A*G*G-ooo-(CH₂)₆NH₂-3′ (SEQ ID NO: 13). SEQ ID NO: 13 has a 5′ terminal —OH. The symbol “o” is an internal C3 spacer, which has the following chemical structure:

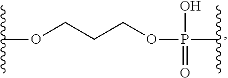 [SMILES: *OCCCOP(*)(=O)O]

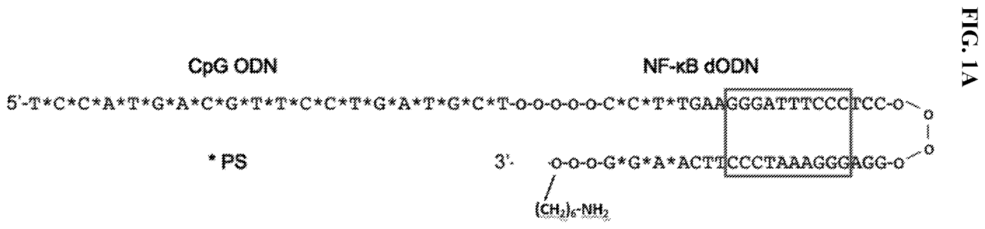

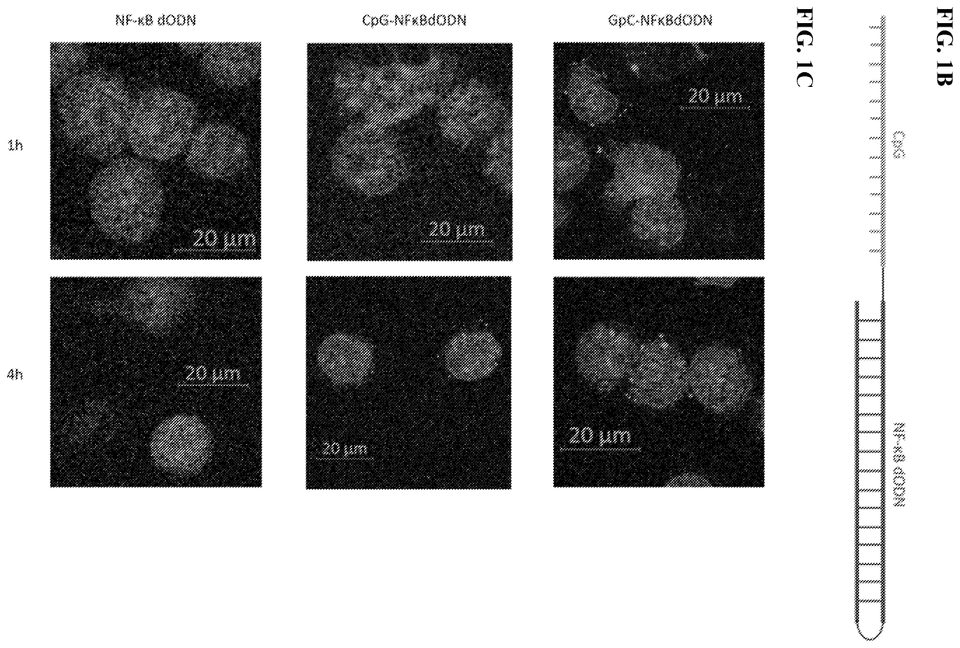

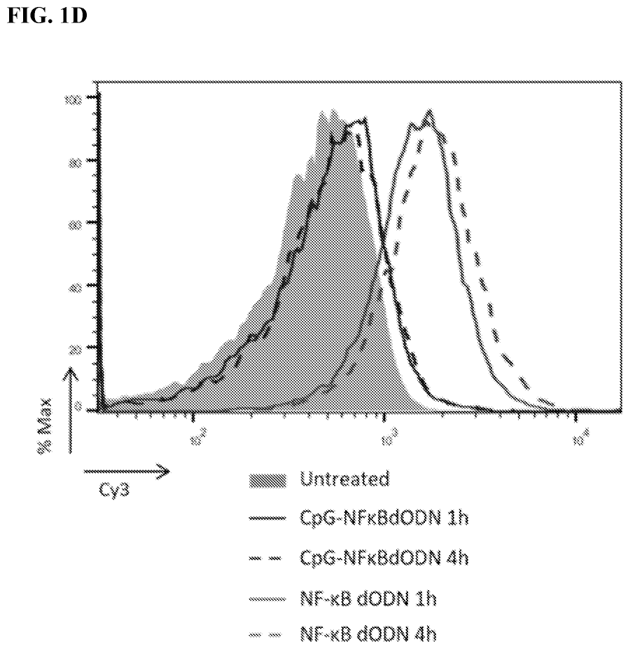

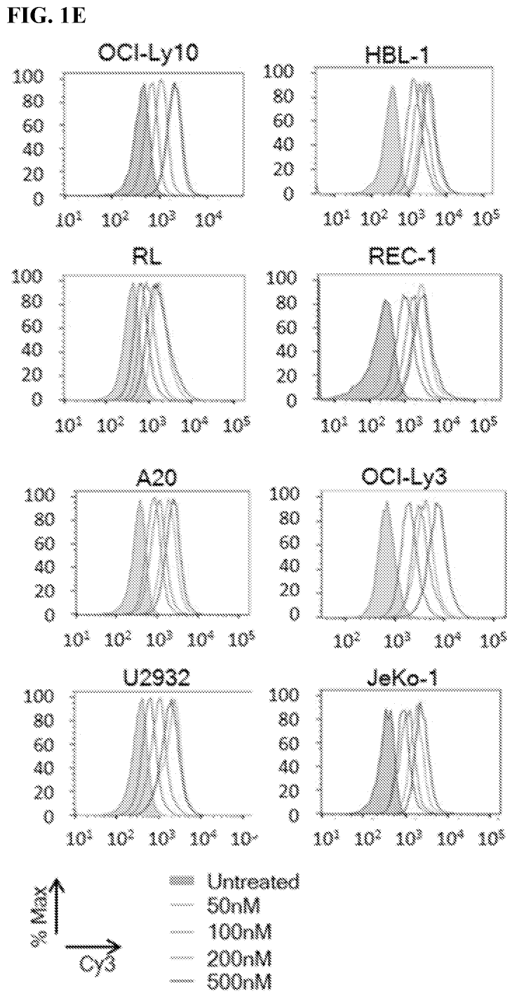

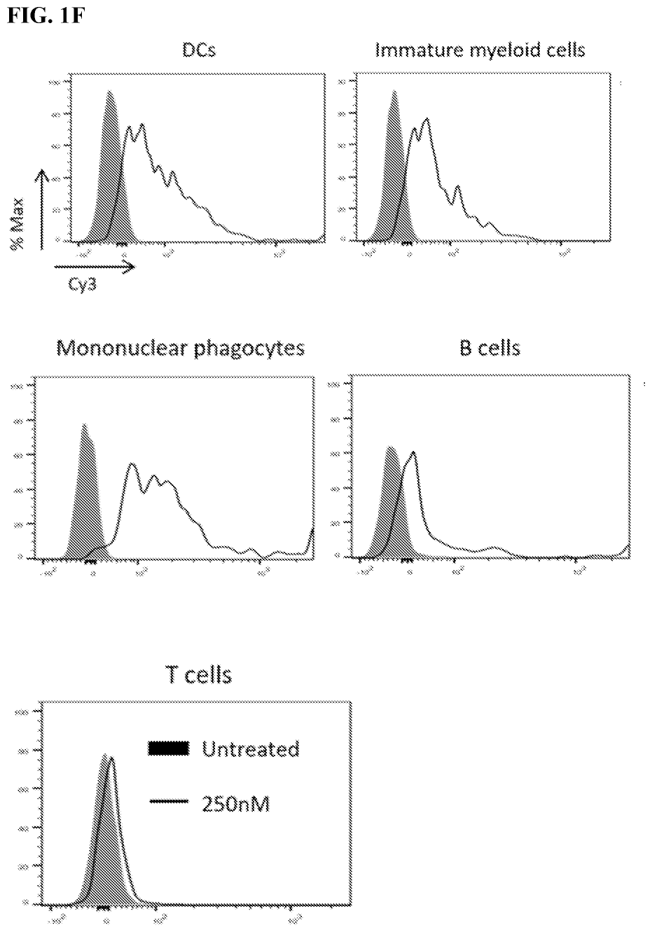

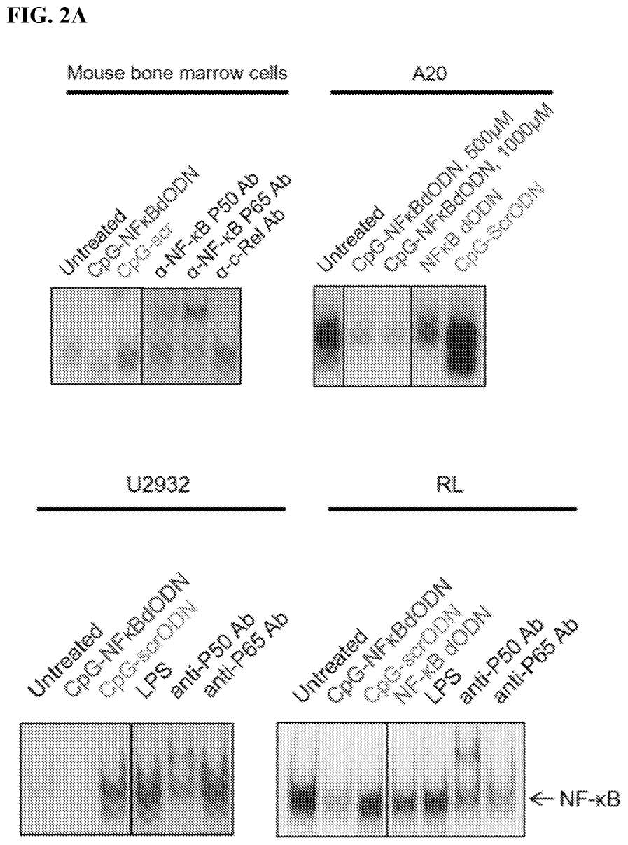

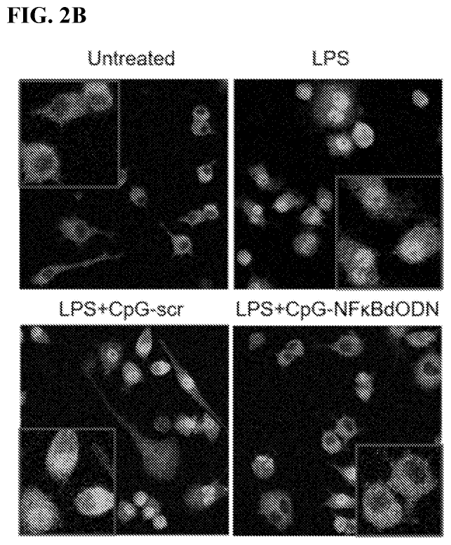

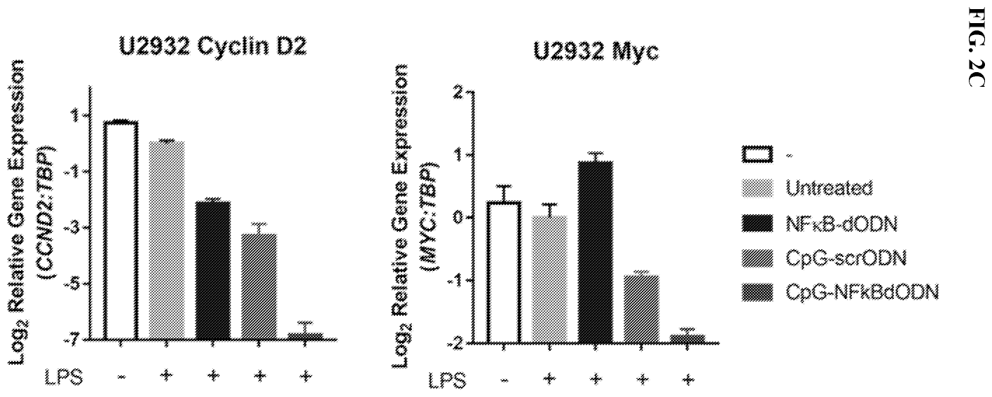

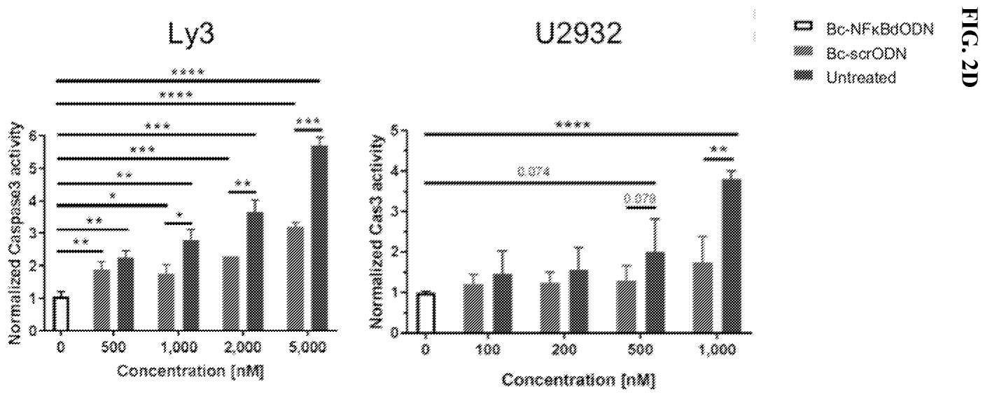

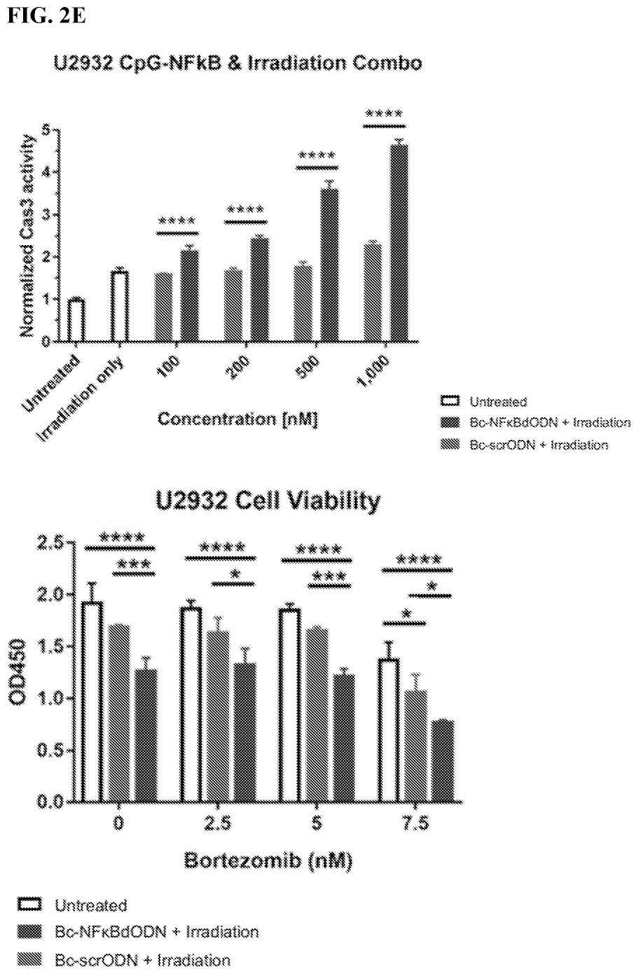

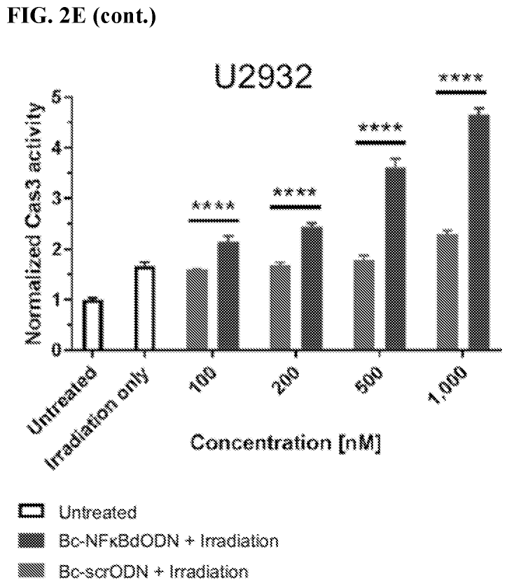

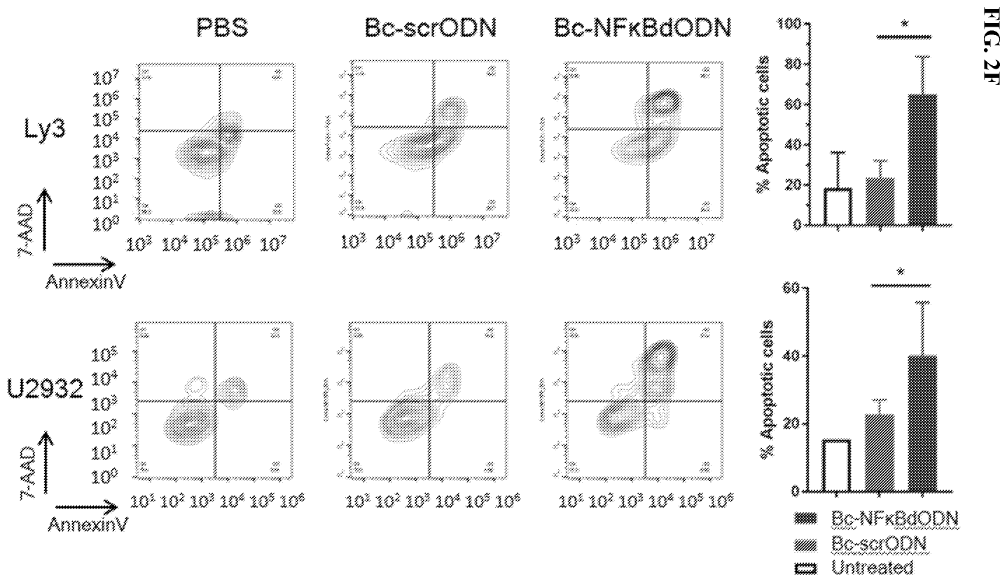

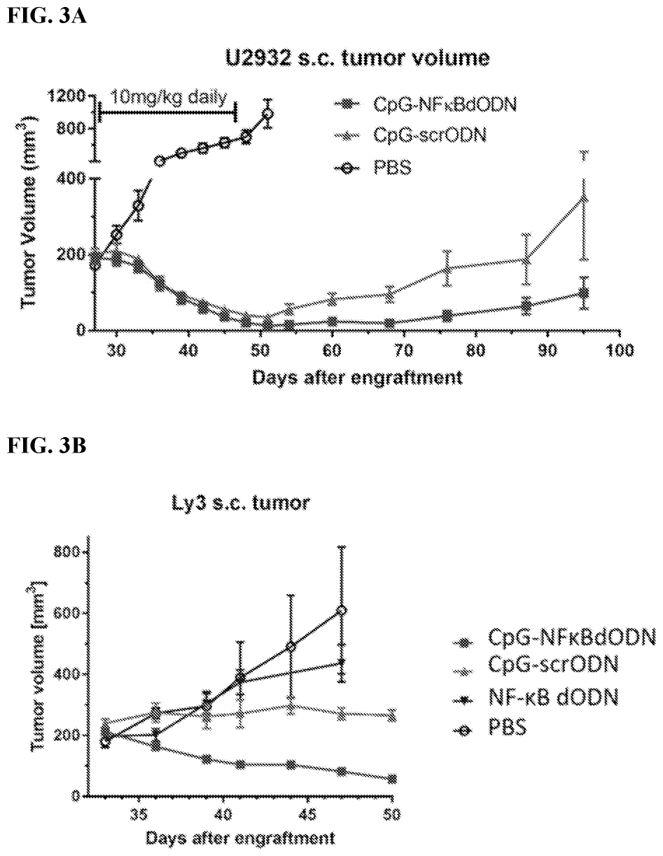

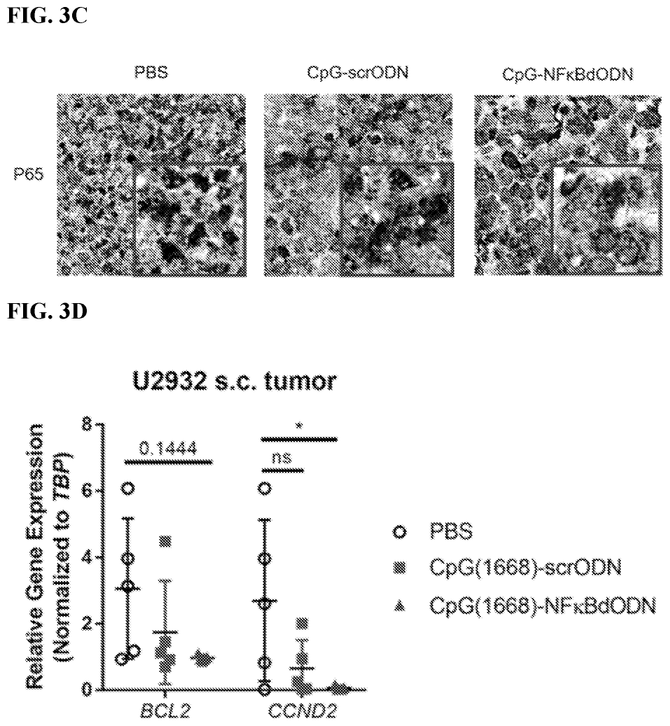

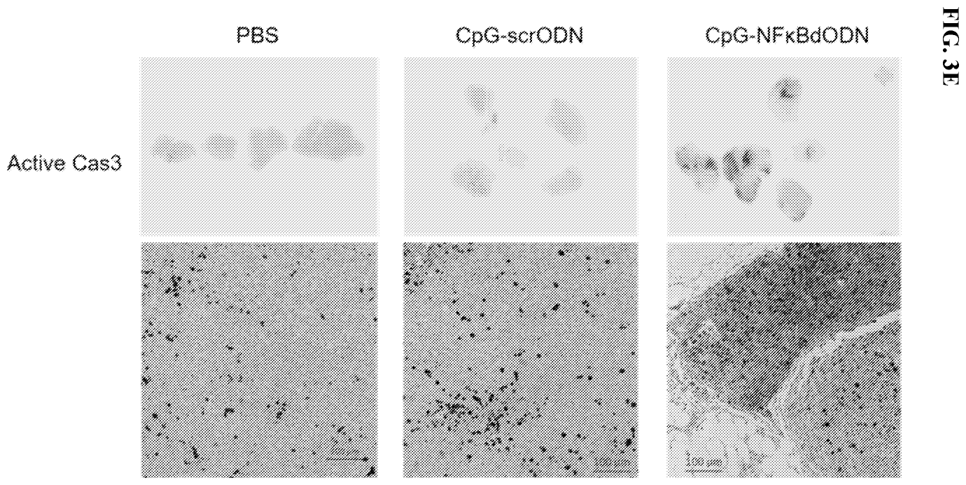

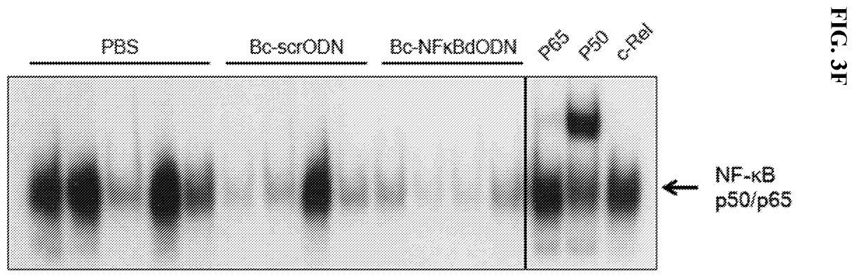

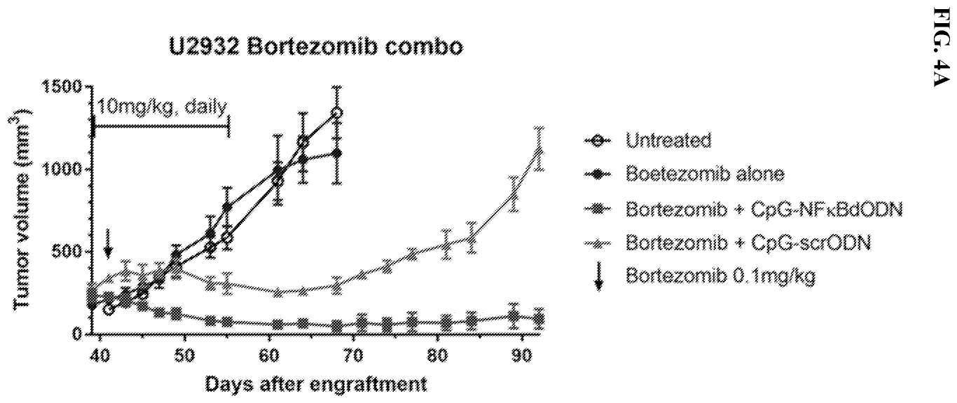

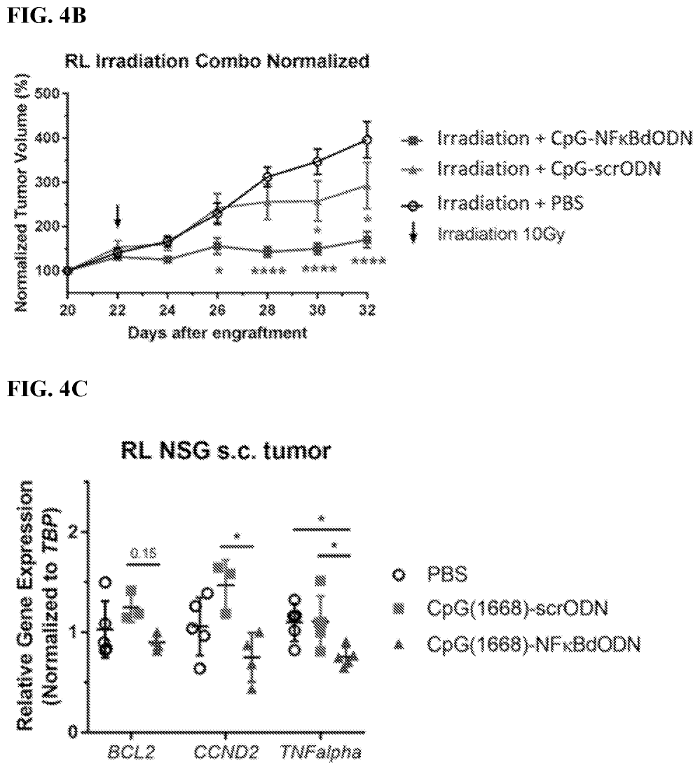

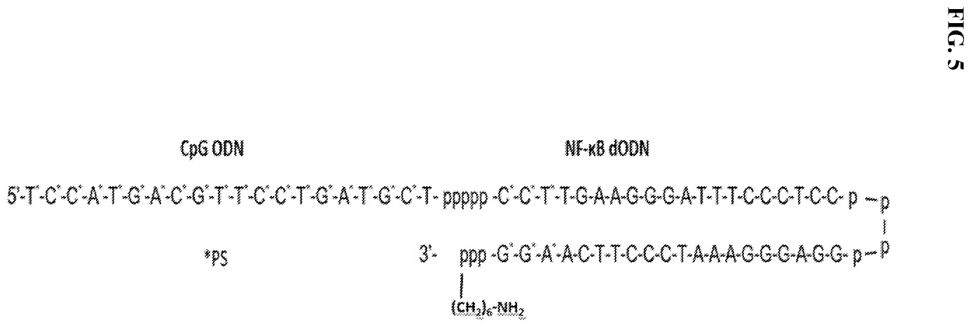

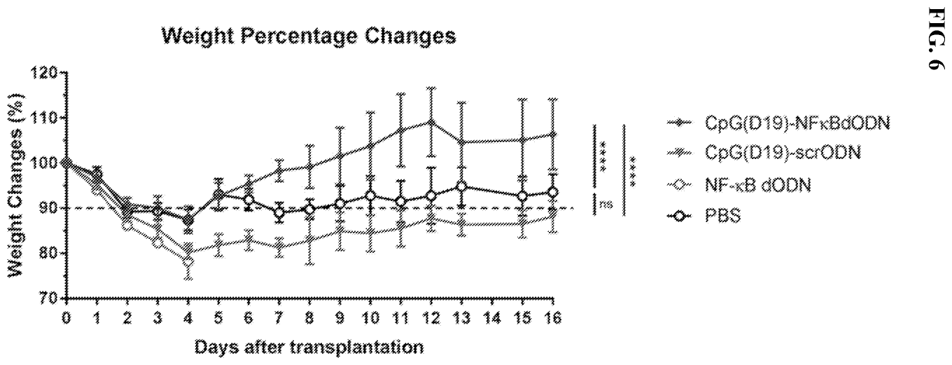

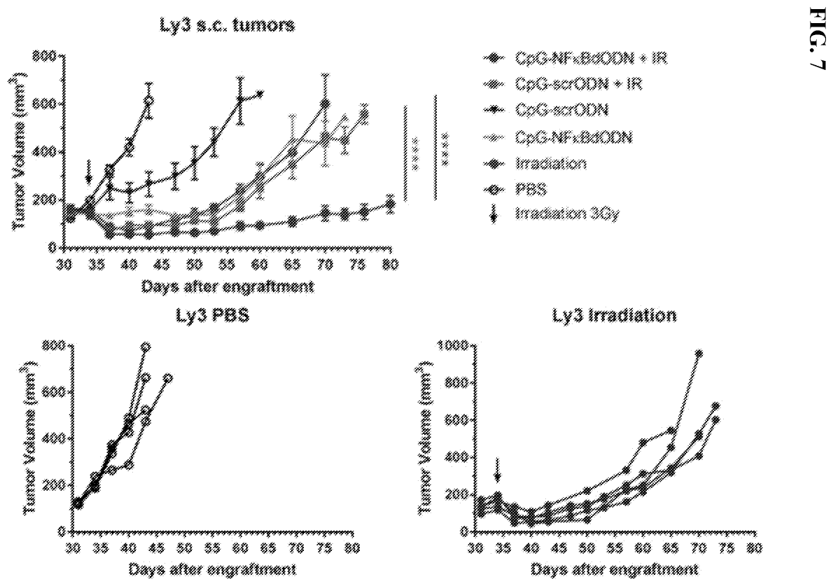

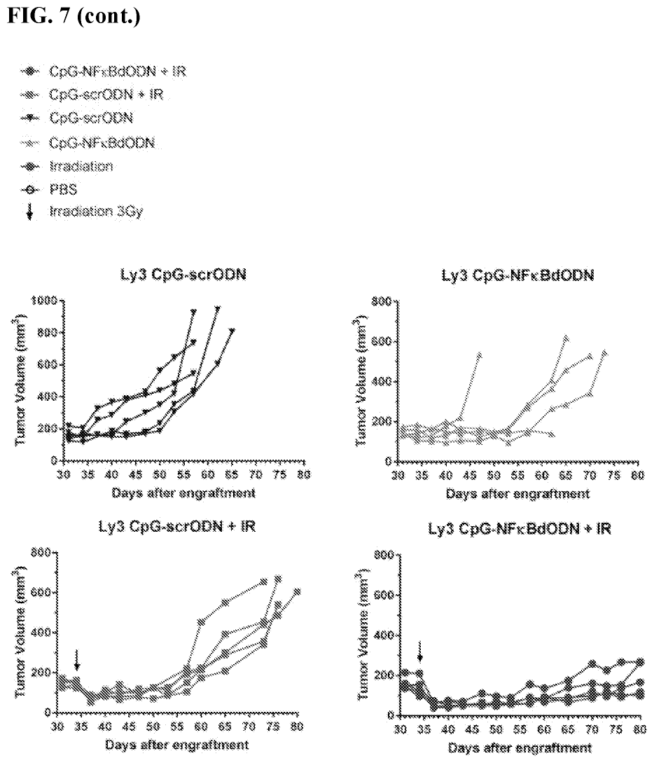

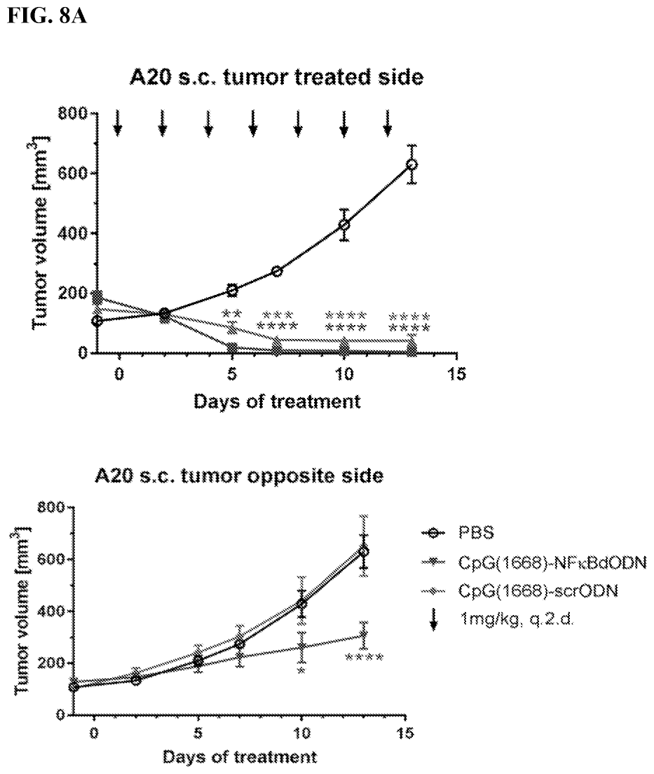

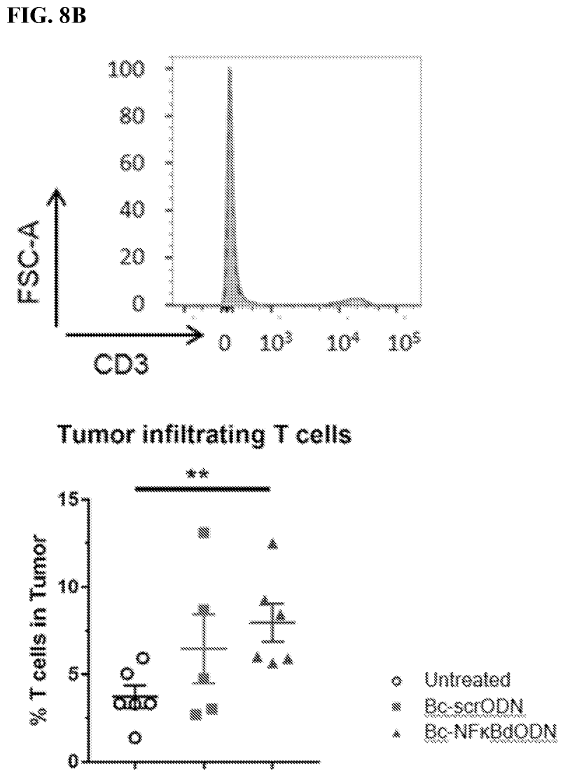

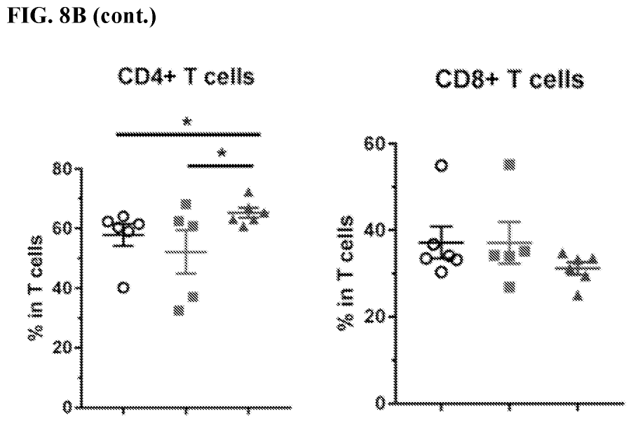

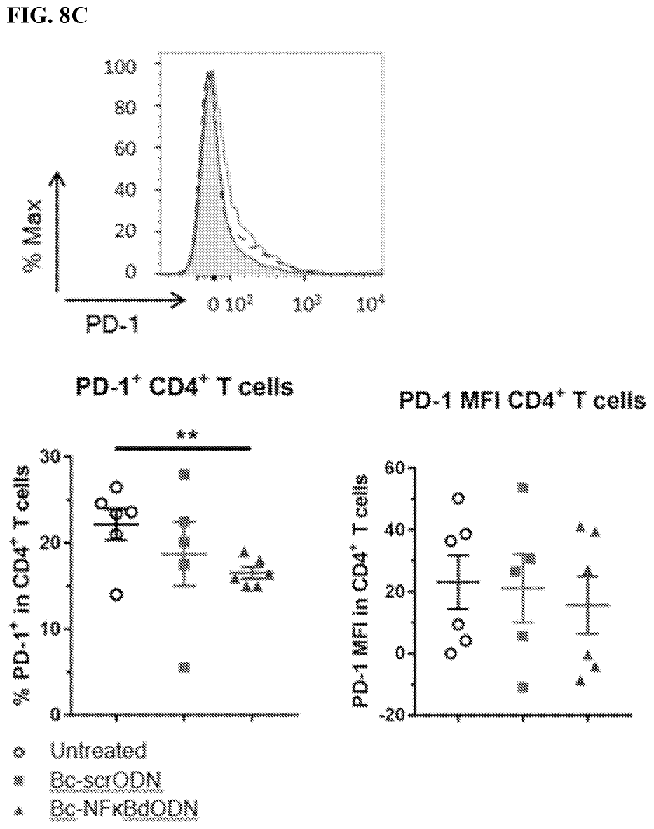

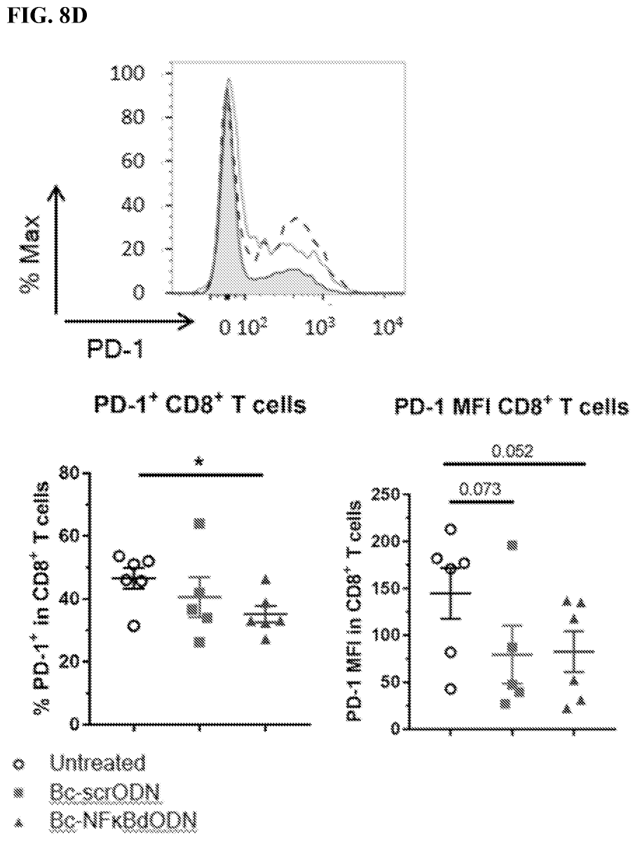

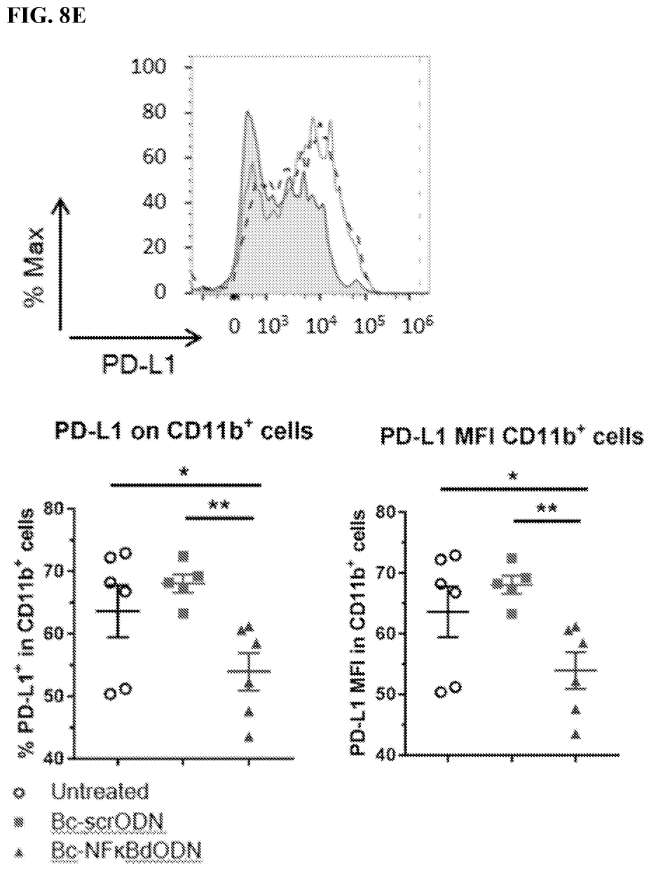

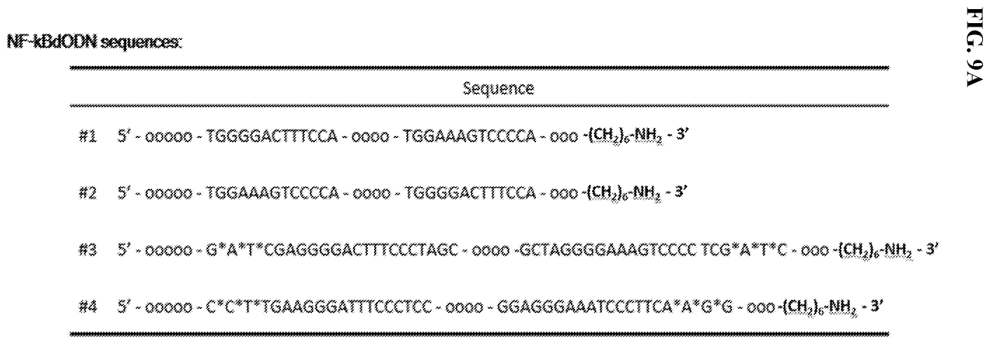

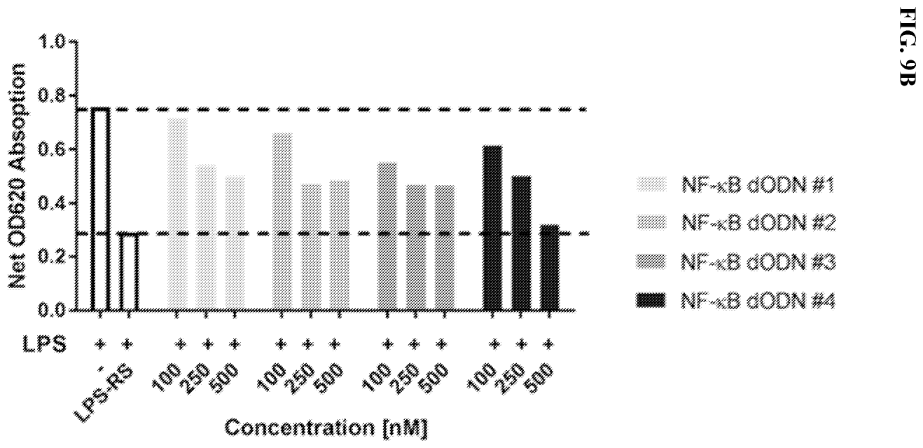

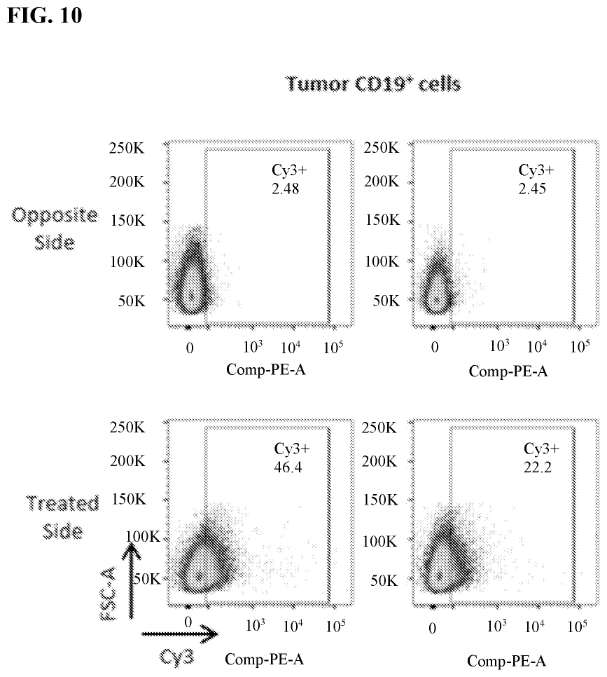

*=phosphorothioation. FIG. 1B: Schematics of CpG-NFκBdODN design. FIG. 1C. Uptake of CpG-NFκBdODN by mouse BCL A20 cells and the uptake was examined by confocal microscopy. FIG. 1D: Mouse A20 lymphoma cells were incubated with 500 nM of Cy3-labeled Bc-NFκBdODN or NF-κB dODN for 1 h or 4 h and the uptake of the constructs were examined by flow cytometry. FIG. 1E: Cy3-labeled Bc-NFκBdODN was incubated with a panel of human and mouse BCL cells at various concentrations for 4 h. The oligonucleotide uptake was assessed by flow cytometry. FIG. 1F: Uptake of Bc-NFκBdODN by dendritic cells (DCs), immature myeloid cells, mononuclear phagocytes, B cells and T cells. The oligonucleotide uptake was assessed by flow cytometry. The y-axis has a scale from 0 to 100 (in increments of 20, e.g., 0, 20, 40, 60, 80 and 100) and the x-axis has a scale from −10² to 10³ (e.g., −10², 0, 10², 10³).

FIGS. 2A-2F. CpG-NFκBdODN inhibits activity of NF-κB and induces apoptosis in primary mouse bone marrow cells and in human and mouse BCL cells. FIG. 2A-2B. CpG-NFκBdODN inhibits DNA-binding by NF-κB as a result of reduced nuclear translocation. FIG. 2C. Reduced downstream NF-κB target gene expression. FIGS. 2D-2E. CpG-NFκBdODN induces apoptosis in human Diffuse Large B-cell Lymphoma (DLBCL) cells. Ly3 and U2932 cells were treated with Bc-NFκBdODN or Bc-scrODN daily for three days and irradiated using 2.5 Gy (Ly3) or 10 Gy (U2932). The induction of cell death was examined by measuring the activity of caspase 3. FIG. 2F: Ly3 and U2932 cells were treated with designated concentration of Bc-NFκBdODN or Bc-scrODN daily for three days, then tested for the caspase 3 activity using Caspase-Glo3/7 Assay System (Promega).

FIGS. 3A-3F. Intratumoral administration of CpG-NFκBdODN triggers regression of human ABC-DLBCL xenotransplants. FIGS. 3A-3B. The antitumor effect of CpG-NFκBdODN vs. control CpG-scrODN in sc-engrafted U2932 (FIG. 3A) and Ly3 (FIG. 3B) lymphoma models. NSG mice were engrafted s.c. with 10⁷ U2932 at abdomen and treated with 10 mg/kg Bc-NFκBdODN or Bc-scrODN intratumorally every day from D28 through D48 (FIG. 3A). NSG mice were engrafted s.c. with 10⁷ Ly3 cells and treated intratumorally using 10 mg/kg Bc-NFκBdODN, Bc-scrODN or PBS from D33 through D47 post engraftment (FIG. 3B). FIGS. 3A-3E. CpG-NFκBdODN inhibits NF-κB nuclear localization (FIG. 3C), target gene expression (FIG. 3D) and induces apoptosis (caspase-3 activity) in U2932 lymphoma cells. FIG. 3F. NSG mice were engrafted s.c. with 10⁷ U2932 at abdomen and treated with 10 mg/kg Bc-NFκBdODN or Bc-scrODN intratumorally every day for three days. Tumor cells were collected and NF-κB activity was detected using EMSA assay.

FIGS. 4A-4C. CpG-NFκBdODN enhances efficacy of radiation therapy or treatment using proteosome inhibitor in vivo. FIG. 4A. CpG-NFκBdODN in combination with bortezomib treatment induces regression of U2932 lymphoma. FIG. 4B. The synergistic effect of CpG-NFκBdODN and single dose tumor irradiation against RL mantle cell lymphoma. NSG mice were engrafted s.c. with 10⁷ RL cells and received two i.t. injections of 10 mg/kg Bc-NFκBdODN, Bc-scrODN or PBS after 10 Gy irradiation on D22, then treated using Bc-NFκBdODN, Bc-scrODN or PBS treatment until D32 (n=5 in each group). FIG. 4C. Changes in expression of NF-κB target genes such as CCND2, BCL2 and TNFA.

FIG. 5. Schematic design of a compound comprising a first nucleic acid sequence capable of binding to Nuclear Factor Kappa-Light-Chain-Enhancer of Activated B Cells (NF-κB) and a second nucleic acid sequence capable of binding a Toll-like receptor (TLR) protein covalently bound to said first nucleic acid. The CpG motif is completely phosphorothioated (PS), while NF-κB dODN has PS modification on 3′- and 5′-ends. 5′ T*C*C*A*T*G*A*C*G*T*T*C*C*T*G*A*T*G*C*T-ppppp-C*C*T*TGA AGG GAT TTC CCT CC-pppp-GG AGG GAA ATC CCT TCA*A*G*G-ppp-(CH₂)₆NH₂ 3′ (SEQ ID NO: 13). The symbol “p” is an internal C3 spacer, which has the following chemical structure:

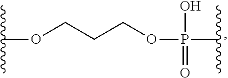 [SMILES: *OCCCOP(*)(=O)O]

*=phosphorothioation. SEQ ID NO: 13 has a 5′ terminal —OH.

FIG. 6. CpG-NFκBdODN suppressed acute and chronic GVHD in mouse allo-HCT models. BALB/c mice (recipient) were irradiated with 850 cGy and received 2.5×10⁶ T-cell depleted bone marrow cells and 1×10⁶ splenocytes from C57BL/6 mice (donor). Recipient mice were treated daily with 10 mg/kg CpG-NFκBdODN starting at 2 days prior to allo-HCT for total of 12 treatments. Mice were monitored for weight loss as an indication of severe inflammatory conditions.

FIG. 7. Targeting NF-kB signaling improves the efficacy of local irradiation in human B-cell lymphoma and mantle cell lymphoma tumor models. FIG. 7: NSG mice were engrafted s.c. with 10⁷ OCI-Ly3 cells and treated intratumorally everyday with 10 mg/kg CpG-NFκBdODN, CpG-scrODN or PBS for two days, followed by irradiation using 3 Gy on day 35. After tumor irradiation, oligonucleotide injections were continued for three weeks (n=5 in each group).

FIGS. 8A-8E. Bc-NFκBdODN induces immune-mediated tumor regression. A20 cells were engrafted s.c. at both sides of abdomen in BALB/c mice and one side of the tumors were treated every other day with 1 mg/kg Bc-NFκBdODN for 2 weeks. FIG. 8A: Tumor progression was analyzed using caliper measurements (n=9 in each group). FIGS. 8B-8E: Tumor from the opposite site were collected at the end of the experiment and processed into single cell suspension. Tumor infiltrating immune cells profiling were analyzed using flow cytometry for total and CD4⁺/CD8⁺ T cells (FIG. 8B), PD-1 expression on CD4⁺ (FIG. 8C) and CD8⁺ T cells (FIG. 8D) and PD-L1 expression on CD11b⁺ cells (FIG. 8E). (n=6 in each group, shown are representative flow cytometry figures).

FIGS. 9A-9B. Design of CpG-NFκBdODN conjugates for selective targeting of myeloid immune cells and B cell lymphoma cells. FIG. 9A: NF-κB decoy oligonucleotide sequences tested in this study. #1-5′-ooooo-TGGGGACTTTCCA-oooo-TGGAAAGTCCCCA-ooo-(CH₂)₆NH₂-3′ (SEQ ID NO: 9). #2-5′-ooooo-TGGAAAGTCCCCA-oooo-TGGGGACTTTCCA-ooo-(CH₂)₆NH₂-3′ (SEQ ID NO: 10). #3-5′-ooooo-G*A*T*CGAGGGGACTTTCCCTAGC-oooo-GCTAGGGAAAGTCCCC TCG*A*T*C-ooo-(CH₂)₆NH₂-3′ (SEQ ID NO: 11). #4-5′-ooooo-C*C*T*TGAAGGGATTTCCCT CC-oooo-GGAGGGAAATCCCTTCA*A*G*G-ooo-(CH₂)₆NH₂-3′ (SEQ ID NO: 12). The symbol “o” is an internal C3 spacer, which has the following chemical structure:

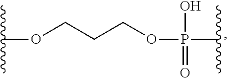 [SMILES: *OCCCOP(*)(=O)O]

*=phosphorothioation. FIG. 9B: RAWBlue cells were treated every day for three days with various concentrations of NF-κB dODNs or 1,000 ng/mL LPS-RS (LPS antagonist) as negative control, then stimulated using 100 ng/mL LPS overnight. SEAP activity was measured by OD620 absorption. Shown are mean values of duplicate samples.

FIG. 10. A20 cells were engrafted s.c. at both sides of abdomen in BALB/c mice and one side of the tumors were injected with 1 mg/kg Cy3-labeled Bc-NFκBdODN. Both sides of tumors were collected 3 h post injection to detect internalization of Cy3-labeled Bc-NFκBdODN.

In all figures and throughout the specification, the terms CpG(1668)-scrODN, CpG-scr, CpG-scrODN, and Bc-ScrODN are equivalent. In all figures and throughout the specification, the terms CpG(1668)-NFκBdODN, CpG-NFκBdODN, and Bc-NFκBdODN are equivalent and they refer to SEQ ID NO: 13.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

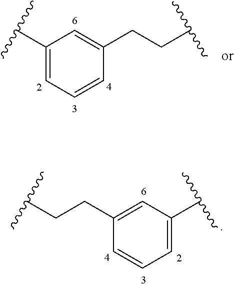 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃ unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
   (i) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃ unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
      (a) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Where a moiety is substituted (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene), the moiety is substituted with at least one substituent (e.g., a substituent group, a size-limited substituent group, or lower substituent group) and each substituent is optionally different. Additionally, where multiple substituents are present on a moiety, each substituent may be optionally differently.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc.;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g. phosphines) to form, for example, phosphate diester bonds;
(n) azides coupled to alkynes using copper catalyzed cycloaddition click chemistry; and
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D} etc., wherein each of R^{13A}, R^{13B}, R^{13C}, R^{13D}, etc. is defined within the scope of the definition of R¹³ and optionally differently.

A “detectable agent” or “detectable moiety” is a substance, agent, moiety, element, compound, or composition detectable by appropriate means such as spectroscopic, photochemical, biochemical, immunochemical, chemical, magnetic resonance imaging, or other physical means. For example, useful detectable agents include ¹⁸F, ³²P ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu ¹⁷⁷Lu, ¹⁸⁶Re, ¹⁸⁸Re, ¹⁸⁹Re, ¹⁹⁴Ir ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition. Detectable moieties also include any of the above compositions encapsulated in nanoparticles, particles, aggregates, coated with additional compositions, derivatized for binding to a targeting agent (e.g. compound described herein). Any method known in the art for conjugating an oligonucleotide or protein to the label may be employed, e.g., using methods described in Hermanson, Bioconjugate Techniques 1996, Academic Press, Inc., San Diego.

Radioactive substances (e.g., radioisotopes) that may be used as imaging and/or labeling agents in accordance with the embodiments of the disclosure include, but are not limited to, ¹⁸F, ³²P ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu ¹⁷⁷Lu, ¹⁸⁶Re, ¹⁸⁸Re, ¹⁸⁹Re, ¹⁹⁴Ir ¹⁹⁸Au, ¹⁹⁹Au, ²¹¹At, ²¹¹Pb, ²¹²Bi, ²¹²Pb, ²¹³Bi, ²²³Ra, ²²⁵Ac, Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M); (see, e.g., Creighton, Proteins (1984)).

Percentage of sequence identity is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleotide” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. In certain embodiments, the nucleic acids herein contain phosphodiester bonds. In other embodiments, nucleic acid analogs are included that may have alternate backbones (e.g. phosphodiester derivatives), including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Nucleotides can be peptide nucleic acids (PNAs), non-ribose backbones including phosphorodiamidate morpholino oligomers and locked nucleic acid (LNA), xeno nucleic acid, ribonucleotides, deoxyribonucleotides, or modified versions thereof. Other analog nucleic acids include those with positive backbones; non-ionic backbones, and modified sugars, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, or other phosphodiester derivatives, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

“Nucleic acid”, “polynucleotide,” “oligonucleotide,” “oligo”, “nucleotide”, or the like also encompass nucleotide analogs or modified backbone residues or linkages or nucleic acids containing known nucleotide analogs or modified backbone residues or linkages, which are synthetic, naturally occurring, and non-naturally occurring, which have similar binding properties as the reference nucleotide or nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “VL” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat cancer (e.g. non-Hodgkin's lymphoma, B-cell lymphoma (BCL), Mantle cell lymphoma (MCL), Diffuse large B-cell lymphoma (DLBCL), activated B-cell subtype Diffuse large B-cell lymphoma (ABC-DBLCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), Chronic lymphocytic leukemia (CLL), or acute lymphoblastic leukemia (ALL)). For example certain methods herein treat cancer by decreasing or reducing or preventing the occurrence, growth, metastasis, or progression of cancer; or treat cancer by decreasing a symptom of cancer. Symptoms of cancer (e.g. non-Hodgkin's lymphoma, B-cell lymphoma (BCL), Mantle cell lymphoma (MCL), Diffuse large B-cell lymphoma (DLBCL), activated B-cell subtype Diffuse large B-cell lymphoma (ABC-DBLCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), Chronic lymphocytic leukemia (CLL), or acute lymphoblastic leukemia (ALL)) would be known or may be determined by a person of ordinary skill in the art. The term “treating” and conjugations thereof, include prevention of an injury, pathology, condition, or disease (e.g. preventing the development of one or more symptoms of cancer (e.g. non-Hodgkin's lymphoma, B-cell lymphoma (BCL), Mantle cell lymphoma (MCL), Diffuse large B-cell lymphoma (DLBCL), activated B-cell subtype Diffuse large B-cell lymphoma (ABC-DBLCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), Chronic lymphocytic leukemia (CLL), or acute lymphoblastic leukemia (ALL)). Treating or treatment as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

An “effective amount” is an amount sufficient to accomplish a stated purpose (e.g. achieve the effect for which it is administered, treat a disease, reduce enzyme activity, increase enzyme activity, reduce transcriptional activity, increase transcriptional activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein (e.g. transcription factor) relative to the absence of the antagonist. An “activity increasing amount,” as used herein, refers to an amount of agonist (activator) required to increase the activity of an enzyme or protein (e.g. transcription factor) relative to the absence of the agonist. A “function disrupting amount,” as used herein, refers to the amount of antagonist (inhibitor) required to disrupt the function of an enzyme or protein (e.g. transcription factor) relative to the absence of the antagonist. A “function increasing amount,” as used herein, refers to the amount of agonist (activator) required to increase the function of an enzyme or protein (e.g. NFKB) relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g cancer (e.g. non-Hodgkin's lymphoma, B-cell lymphoma (BCL), Mantle cell lymphoma (MCL), Diffuse large B-cell lymphoma (DLBCL), activated B-cell subtype Diffuse large B-cell lymphoma (ABC-DBLCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), or Chronic lymphocytic leukemia (CLL))) means that the disease (e.g. cancer (e.g. non-Hodgkin's lymphoma, B-cell lymphoma (BCL), Mantle cell lymphoma (MCL), activated B-cell subtype Diffuse large B-cell lymphoma (ABC-DBLCL), Diffuse large B-cell lymphoma (DLBCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), Chronic lymphocytic leukemia (CLL), or acute lymphoblastic leukemia (ALL)) or infectious disease (e.g. ZIKA virus infection, herpes virus infection associated disease or hepatitis virus infection associated disease or HIV infection associated disease) is caused by (in whole or in part), or a symptom of the disease is caused by (in whole or in part) the substance or substance activity or function.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme (e.g. NF-κB). In some embodiments, contacting includes allowing a compound described herein to interact with a protein or enzyme that is involved in a signaling pathway (e.g. NF-κB pathway).

As defined herein, the term “inhibition”, “inhibit”, “inhibiting” and the like in reference to a protein-inhibitor (e.g. antagonist) interaction means negatively affecting (e.g. decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein. In some embodiments, inhibition refers to a decrease in the activity of a signal transduction pathway or signaling pathway (e.g. NF-κB activated pathway). Thus, inhibition may include, at least in part, partially or totally decreasing stimulation, decreasing or reducing activation, or inactivating, desensitizing, or down-regulating signal transduction or enzymatic activity or the amount of a protein increased in a disease (e.g. level of a NF-κB) activity or protein or level, wherein each is associated with cancer (e.g. non-Hodgkin's lymphoma, B-cell lymphoma (BCL), Mantle cell lymphoma (MCL), Diffuse large B-cell lymphoma (DLBCL), activated B-cell subtype Diffuse large B-cell lymphoma (ABC-DBLCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), Chronic lymphocytic leukemia (CLL), or acute lymphoblastic leukemia (ALL)) or graft-vs-host disease. Inhibition may include, at least in part, partially or totally decreasing stimulation, decreasing or reducing activation, or deactivating, desensitizing, or down-regulating signal transduction or enzymatic activity or the amount of a protein (e.g. NF-κB).

As defined herein, the term “activation”, “activate”, “activating” and the like in reference to a protein-activator (e.g. agonist) interaction means positively affecting (e.g. increasing) the activity or function of the protein (e.g. NF-κB), or a component of a pathway including a NF-κB relative to the activity or function of the protein in the absence of the activator (e.g. compound described herein). Thus, activation may include, at least in part, partially or totally increasing stimulation, increasing or enabling activation, or activating, sensitizing, or up-regulating signal transduction or enzymatic activity or the amount of a protein decreased in a disease (e.g. level of NF-κB or protein associated with cancer (e.g. non-Hodgkin's lymphoma, B-cell lymphoma (BCL), Mantle cell lymphoma (MCL), Diffuse large B-cell lymphoma (DLBCL), activated B-cell subtype Diffuse large B-cell lymphoma (ABC-DBLCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), or Chronic lymphocytic leukemia (CLL), or acute lymphoblastic leukemia (ALL)), infectious disease (e.g., a viral disease (e.g. herpesvirus infection associated disease or hepatitis virus infection associated disease or HIV infection associated disease), an autoimmune disease, an inflammatory disease, or graft-versus-host disease. Activation may include, at least in part, partially or totally increasing stimulation, increasing or enabling activation, or activating, sensitizing, or up-regulating signal transduction or enzymatic activity or the amount of a protein (e.g. NF-κB), protein downstream of NF-κB, a protein activated or upregulated by NF-κB) that may modulate the level of another protein or increase cell survival (e.g. increase in NF-κB) activity may increase cell survival in cells that may or may not have an increase in NF-κB) activity relative to a non-disease control).

The term “modulator” refers to a composition that increases or decreases the level of a target molecule or the function of a target molecule (e.g. NF-κB) or transcriptional activation or NF-κB. In some embodiments, a modulator of NF-κB pathway is a compound that reduces the severity of one or more symptoms of a disease associated with NF-κB, for example cancer (e.g. non-Hodgkin's lymphoma, B-cell lymphoma (BCL), Mantle cell lymphoma (MCL), Diffuse large B-cell lymphoma (DLBCL), activated B-cell subtype Diffuse large B-cell lymphoma (ABC-DBLCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), or Chronic lymphocytic leukemia (CLL), or acute lymphoblastic leukemia (ALL)) or graft-vs-host disease, or a disease that is not caused by NF-κB pathway but may benefit from modulation of NF-κB pathway activity (e.g. decreasing in level or level of activity of NF-κB). In embodiments, a modulator of NF-κB or NF-κB pathway is an anti-cancer agent. In embodiments, a modulator of NF-κB pathway is an anti-viral agent.

“Patient” or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a mammal. In some embodiments, a patient is a mouse. In some embodiments, a patient is an experimental animal. In some embodiments, a patient is a rat. In some embodiments, a patient is a test animal.

“Disease” or “condition” refer to a state of being or health status of a patient or subject capable of being treated with a compound, pharmaceutical composition, or method provided herein. In some embodiments, the disease is a disease related to (e.g. caused by) an increase in the level of a NF-κB, NF-κB pathway activity, or pathway activated by a NF-κB. In some embodiments, the disease is cancer (e.g. non-Hodgkin's lymphoma, B-cell lymphoma (BCL), Mantle cell lymphoma (MCL), Diffuse large B-cell lymphoma (DLBCL), activated B-cell subtype Diffuse large B-cell lymphoma (ABC-DBLCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), Chronic lymphocytic leukemia (CLL), or acute lymphoblastic leukemia (ALL)). In embodiments, the disease is a viral disease (e.g. herpesvirus infection associated disease or hepatitis virus infection associated disease or HIV infection associated disease) associated with NF-κB-dependent immunosuppression. In embodiments, the disease is an autoimmune disease. In embodiments, the disease is an infectious disease. In embodiments, the disease is an inflammatory disease. In embodiments, the disease is a graft-versus-host disease.

Examples of diseases, disorders, or conditions include, but are not limited to, cancer (e.g. prostate cancer, castration-resistant prostate cancer, breast cancer, triple negative breast cancer, glioblastoma, ovarian cancer, lung cancer, squamous cell carcinoma (e.g head, neck, or esophagus), colorectal cancer, leukemia, acute myeloid leukemia, lymphoma, B cell lymphoma, or multiple myeloma). In some instances, “disease” or “condition” refers to cancer. In some further instances, “cancer” refers to human cancers and carcinomas, sarcomas, adenocarcinomas, lymphomas, leukemias, melanomas, etc., including solid and lymphoid cancers, kidney, breast, lung, bladder, colon, ovarian, prostate, pancreas, stomach, brain, head and neck, skin, uterine, testicular, glioma, esophagus, liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), and/or multiple myeloma. In some further instances, “cancer” refers to lung cancer, breast cancer, ovarian cancer, leukemia, lymphoma, melanoma, pancreatic cancer, sarcoma, bladder cancer, bone cancer, brain cancer, cervical cancer, colon cancer, esophageal cancer, gastric cancer, liver cancer, head and neck cancer, kidney cancer, myeloma, thyroid cancer, prostate cancer, metastatic cancer, or carcinoma.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, acute lymphoblastic leukemia (ALL), chronic lymphocytic leukemia (CLL), acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, acute myeloid leukemia (AML), chronic myeloid leukemia (CML), leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myelodysplastic syndrome (MDS), myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma (MCL), follicular lymphoma, marginal zone B-cell lymphoma (MZL), mucosa-associated lymphatic tissue lymphoma (MALT), extranodal lymphoma, nodal (monocytoid B-cell) lymphoma, splenic lymphoma, diffuse large cell B-lymphoma (DLBCL), activated B-cell subtype diffuse large B-cell lymphoma (ABC-DBLCL), germinal center B-cell like diffuse large B-cell lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungocides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “autoimmune disease” or “autoimmune disorder” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), graft-vs-host disease, Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Systemic lupus erythematosus (SLE), Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “neurodegenerative disease” or “neurodegenerative disorder” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

As used herein, the term “inflammatory disease” refers to a disease or condition characterized by aberrant inflammation (e.g. an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). In embodiments, administration includes direct administration to a tumor. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent or chemotherapeutic). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

Pharmaceutical compositions provided by the present invention include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g. NF-κB), and/or reducing, eliminating, or slowing the progression of disease symptoms (e.g. symptoms of cancer (e.g., non-Hodgkin's lymphoma, B-cell lymphoma (BCL), Mantle cell lymphoma (MCL), Diffuse large B-cell lymphoma (DLBCL), activated B-cell subtype Diffuse large B-cell lymphoma (ABC-DBLCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), Chronic lymphocytic leukemia (CLL), or acute lymphoblastic leukemia (ALL)), or an infectious disease (e.g., a viral disease (e.g. herpesvirus infection associated disease or hepatitis virus infection associated disease or HIV infection associated disease), an autoimmune disease, an inflammatory disease, or graft-versus-host disease. Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer (e.g. prostate cancer, castration-resistant prostate cancer, breast cancer, triple negative breast cancer, glioblastoma, ovarian cancer, lung cancer, squamous cell carcinoma (e.g head, neck, or esophagus), colorectal cancer, leukemia, acute myeloid leukemia, lymphoma, B cell lymphoma, or multiple myeloma)), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating cancer (e.g. non-Hodgkin's lymphoma, B-cell lymphoma (BCL), Mantle cell lymphoma (MCL), Diffuse large B-cell lymphoma (DLBCL), activated B-cell subtype Diffuse large B-cell lymphoma (ABC-DBLCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), Chronic lymphocytic leukemia (CLL), or acute lymphoblastic leukemia (ALL)), or with other active agents known to be useful in treating infectious diseases, graft-versus-host disease, an autoimmune disease, an inflammatory disease, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for cancer (e.g. non-Hodgkin's lymphoma, B-cell lymphoma (BCL), Mantle cell lymphoma (MCL), Diffuse large B-cell lymphoma (DLBCL), activated B-cell subtype Diffuse large B-cell lymphoma (ABC-DBLCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), Chronic lymphocytic leukemia (CLL), or acute lymphoblastic leukemia (ALL)) such as surgery or with other treatments known to be useful in treating an infectious disease, graft-versus-host disease, an autoimmune disease, an inflammatory disease.

“Anti-viral agent” is used in accordance with its plain ordinary meaning and refers to a composition (e.g. compound, drug, antagonist, inhibitor, modulator) having anti-infective properties or the ability to inhibit the growth or proliferation of virus. In some embodiments, an anti-viral agent is an agent identified herein having utility in methods of treating viral disease (e.g. herpesvirus infection associated disease or hepatitis virus infection associated disease or HIV infection associated disease). In some embodiments, an anti-viral agent is an agent approved by the FDA or similar regulatory agency of a country other than the USA, for treating viral disease (e.g. herpesvirus infection associated disease or hepatitis virus infection associated disease or HIV infection associated disease). Examples of anti-viral agents are well known in the art and include agents for treating herpesvirus infection associated disease, hepatitis virus infection associated disease, and HIV infection associated disease. In some embodiments, an anti-viral agent is an agent identified herein having utility in methods of treating viral disease (e.g. Zika virus infection).

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002), mTOR inhibitors, antibodies (e.g., rituxan), 5-aza-2′-deoxycytidine, doxorubicin, vincristine, etoposide, gemcitabine, imatinib (Gleevec®), geldanamycin, 17-N-Allylamino-17-Demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-1 receptor inhibitor; interferon agonists; interferons; interleukins; letrozole; leukemia inhibiting factor; leukocyte alpha interferon; leuprolide+estrogen+progesterone; leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein kinase C inhibitors, protein tyrosine phosphatase inhibitors; purine nucleoside phosphorylase inhibitors; ras farnesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

Additionally, the compounds described herein can be co-administered with conventional immunotherapeutic agents including, but not limited to, immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, 90 or 131I, etc.).

In a further embodiment, the compounds described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y ⁸⁷Y, ⁹⁰Y ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

An amino acid residue in a protein “corresponds” to a given residue when it occupies the same essential structural position within the protein as the given residue.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 10 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

The phrase “selectively (or specifically) hybridizes to” refers to the binding, duplexing, or hybridizing of a molecule only to a particular nucleotide sequence with a higher affinity, e.g., under more stringent conditions, than to other nucleotide sequences (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

MRI can be used to non-invasively acquire tissue images with high resolution. Paramagnetic agents or USPIO nanoparticles or aggregates thereof enhance signal attenuation on T₂-weighted magnetic resonance images, and conjugation of such nanoparticles to binding ligands permits the detection of specific molecules at the cellular level. For example, MRI with nanoparticle detection agents can detect small foci of cancer. See e.g., Y. W. Jun et al., 2005, J. Am. Chern. Soc. 127:5732-5733; Y. M. Huh et al., 2005, J. Am. Chem. Soc. 127:12387-12391. Contrast-enhanced MRI is well-suited for the dynamic non-invasive imaging of macromolecules or of molecular events, but it requires ligands that specifically bind to the molecule of interest. J. W. Bulte et al., 2004, NMR Biomed. 17:484-499. Fluorescent dyes and fluorophores (e.g. fluorescein, fluorescein isothiocyanate, and fluorescein derivatives) can be used to non-invasively acquire tissue images with high resolution, with for example spectrophotometry, two-photon fluorescence, two-photon laser microscopy, or fluorescence microscopy (e.g. of tissue biopsies). MRI can be used to non-invasively acquire tissue images with high resolution, with for example paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, other nanoparticle contrast agents. MRI can be used to non-invasively acquire tissue images with high resolution, with for example Gadolinium, including liposomes or other delivery vehicles containing Gadolinium chelate (“Gd-chelate”) molecules. Positron emission tomography (PET), PET/computed tomography (CT), single photon emission computed tomography (SPECT), and SPECT/CT can be used to non-invasively acquire tissue images with high resolution, with for example radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia. Ultrasound (ultrasonography) and contrast enhanced ultrasound (contrast enhanced ultrasonography) can be used to non-invasively acquire tissue images with high resolution, with for example biocolloids or microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.). X-ray imaging (radiography) or CT can be used to non-invasively acquire tissue images with high resolution, with for example iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, or gold nanoparticle aggregates. These detection methods and instruments and detectable moieties capable of being measured or detected by the corresponding method are non-limiting examples.

As used herein, the term “ultrasmall superparamagnetic iron oxide nanoparticle” or “USPIO nanoparticle” refers to superparamagnetic iron oxide particles ranging from 1 to 50 nm in diameter, more typically between 5 and 40 nm in diameter (excluding any coating applied after synthesis). USPIO nanoparticles are commonly made of maghemite (Fe₂O₃) or magnetite (Fe₃O₄) having crystal-containing regions of unpaired spins. Those magnetic domains are disordered in the absence of a magnetic field, but when a field is applied (i.e., while taking an MRI), the magnetic domains align to create a magnetic moment much greater than the sum of the individual unpaired electrons without resulting in residual magnetization of the particles.

The term “TLR” refers to a toll-like receptor protein and homologs thereof. There are 13 toll-like receptors, referred to as “TLR1”, “TLR2”, “TLR3”, “TLR4”, “TLR5”, “TLR6”, “TLR7”, “TLR8”, “TLR9”, “TLR10”, “TLR11”, “TLR12”, and “TLR 13”. In embodiments, “TLR” refers to the human protein. Included in the term “TLR” are the wildtype and mutant forms of the protein. In embodiments, “TLR” refers to the wildtype protein. In embodiments, “TLR” refers to a mutant protein.

The term “TLR9” refers to toll-like receptor 9. In embodiments, “TLR9” refers to the protein associated with Entrez Gene 54106, or UniProt Q9NR96. In embodiments, “TLR9” refers to the human protein. Included in the term “TLR9” are the wildtype and mutant forms of the protein. In embodiments, “TLR9” refers to the wildtype protein. In embodiments, “TLR9” refers to a mutant protein. In embodiments, the reference numbers immediately above refer to the protein, and associated nucleic acids, known as of the date of filing of this application.

As used herein, the term “nucleic acid sequence capable of binding TLR” refers to a nucleic acid sequence optionally including one or more spacers within the sequence, wherein at least a portion of the sequence is a “TLR-binding site nucleic acid sequence”.

As used herein, the term “TLR-binding site nucleic acid sequence” or “TLR-binding nucleic acid substituent” refers to a substituent or moiety capable of binding to a toll-like receptor (“TLR”) or activating a toll-like receptor, including at least one nucleic acid. In embodiments, a TLR-binding nucleic acid substituent is capable of binding a TLR. In embodiments, a TLR-binding nucleic acid substituent is capable of activating a TLR. In embodiments, a TLR-binding nucleic acid substituent is capable of activating a TLR without directly binding the TLR. In embodiments, a TLR-binding nucleic acid substituent is capable of binding a TLR without activating the TLR. In embodiments, a TLR-binding nucleic acid substituent is a nucleic acid. In embodiments, the TLR-binding nucleic acid substituent includes at least one nucleic acid analog. In embodiments, the TLR-binding nucleic acid substituent includes at least one nucleic acid analog having an alternate backbone (e.g. phosphodiester derivative (e.g. phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite), peptide nucleic acid backbone(s), LNA, or linkages). In embodiments, a TLR-binding nucleic acid substituent includes or is DNA. In embodiments, a TLR-binding nucleic acid substituent includes or is RNA. In embodiments, a TLR-binding nucleic acid substituent includes or is a nucleic acid having internucleotide linkages selected from phosphodiesters and phosphodiester derivatives (e.g. phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, O-methylphosphoroamidite, or combinations thereof). In embodiments, a TLR-binding nucleic acid substituent consists of a nucleic acid having internucleotide linkages selected from phosphodiesters and phosphorothioates. In embodiments, a TLR-binding nucleic acid substituent includes or is a nucleic acid having backbone linkages selected from phosphodiesters and phosphorodithioates. In embodiments, a TLR-binding nucleic acid substituent includes or is a nucleic acid having phosphodiester backbone linkages. In embodiments, a TLR-binding nucleic acid substituent includes or is a nucleic acid having phosphorothioate backbone linkages. In embodiments, a TLR-binding nucleic acid substituent includes or is a nucleic acid having phosphorodithioate backbone linkages. In embodiments, a TLR-binding nucleic acid substituent preferentially binds TLR9 over other TLR. In embodiments, a TLR-binding nucleic acid substituent specifically binds TLR9. In embodiments, a TLR-binding nucleic acid substituent preferentially binds TLR3 over other TLR. In embodiments, a TLR-binding nucleic acid substituent specifically binds TLR3. In embodiments, a TLR-binding nucleic acid substituent preferentially binds TLR7 over other TLR. In embodiments, a TLR-binding nucleic acid substituent specifically binds TLR7. In embodiments, a TLR-binding nucleic acid substituent preferentially binds TLR8 over other TLR. In embodiments, a TLR-binding nucleic acid substituent specifically binds TLR8. In embodiments, a TLR-binding nucleic acid substituent specifically binds a cellular subcompartment (e.g. endosome) associated TLR (e.g. TLR3, TLR7, TLR8, or TLR9). In embodiments, a TLR-binding nucleic acid substituent includes or is a G-rich nucleic acid (e.g. about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% G nucleotides; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% G nucleotides). In embodiments, a TLR-binding nucleic acid substituent includes single stranded RNA (including phosphodiester internucleotide linkages, phosphodiester derivative internucleotide linkages, or a combination both). In embodiments, a TLR-binding nucleic acid substituent includes double stranded RNA (including phosphodiester internucleotide linkages, phosphodiester derivative internucleotide linkages, or a combination both) (e.g. poly (I:C). In embodiments, a TLR-binding nucleic acid substituent is a TLR3-binding nucleic acid substituent. In embodiments, a TLR-binding nucleic acid substituent is a TLR7-binding nucleic acid substituent. In embodiments, a TLR-binding nucleic acid substituent is a TLR8-binding nucleic acid substituent. In embodiments, a TLR-binding nucleic acid substituent is a TLR9-binding nucleic acid substituent. In embodiments, a TLR-binding nucleic acid substituent is a TLR-binding DNA substituent. In embodiments, a TLR-binding nucleic acid substituent is a TLR9-binding DNA substituent.

As used herein, the term “TLR-binding DNA substituent” refers to a substituent or moiety capable of binding to a toll-like receptor (“TLR”), including at least one deoxyribonucleic acid. In embodiments, a TLR-binding DNA substituent is a nucleic acid. In embodiments, the TLR-binding DNA substituent includes at least one nucleic acid analog. In embodiments, the TLR-binding DNA substituent includes at least one nucleic acid analog having an alternate backbone (e.g. phosphodiester derivative (e.g. phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite), peptide nucleic acid backbone(s), LNA, or linkages). In embodiments, a TLR-binding DNA substituent includes DNA. In embodiments, all nucleotide sugars in a TLR-binding DNA substituent are deoxyribose (e.g., all nucleotides are DNA). In embodiments, a TLR-binding DNA substituent consists of DNA. In embodiments, a TLR-binding DNA substituent includes or is DNA having internucleotide linkages selected from phosphodiesters and phosphodiester derivatives (e.g. phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, O-methylphosphoroamidite, or combinations thereof). In embodiments, a TLR-binding DNA substituent consists of DNA having internucleotide linkages selected from phosphodiesters and phosphorothioates. In embodiments, a TLR-binding DNA substituent includes or is DNA having backbone linkages selected from phosphodiesters and phosphorodithioates. In embodiments, a TLR-binding DNA substituent includes or is DNA including phosphodiester backbone linkages. In embodiments, a TLR-binding DNA substituent includes or is DNA including phosphorothioate backbone linkages. In embodiments, a TLR-binding DNA substituent includes or is DNA including phosphorodithioate backbone linkages. In embodiments, a TLR-binding DNA substituent preferentially binds TLR9 over other TLR. In embodiments, a TLR-binding DNA substituent specifically binds TLR9. In embodiments, a TLR-binding DNA substituent specifically binds TLR3. In embodiments, a TLR-binding DNA substituent specifically binds TLR7. In embodiments, a TLR-binding DNA substituent specifically binds TLR8. In embodiments, a TLR-binding DNA substituent specifically binds a cellular subcompartment (e.g. endosome) associated TLR (e.g. TLR3, TLR7, TLR8, or TLR9). In embodiments, a TLR-binding DNA substituent includes or is a G-rich oligonucleotide (e.g. about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% G nucleotides; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% G nucleotides). In embodiments, a TLR-binding DNA substituent includes a CpG motif, wherein C and G are nucleotides and p is the phosphate connecting the C and G. In embodiments, the CpG motif is unmethylated. In embodiments, a TLR-binding DNA substituent is a Class A CpG oligodeoxynucleotide (ODN). In embodiments, a TLR-binding DNA substituent is a Class B CpG oligodeoxynucleotide (ODN). In embodiments, a TLR-binding DNA substituent is a Class C CpG oligodeoxynucleotide (ODN). In embodiments, a TLR-binding DNA substituent (e.g., TLR9-binding DNA substituent) consists of deoxyribonucleic acids with A, G, C, or T bases and phosphodiester linkages and/or phosphodiester derivative linkages (e.g., phosphorothioate linkage(s)).

As used herein, the term “CpG motif” refers to a 5′ C nucleotide connected to a 3′ G nucleotide through a phosphodiester internucleotide linkage or a phosphodiester derivative internucleotide linkage. In embodiments, a CpG motif includes a phosphodiester internucleotide linkage. In embodiments, a CpG motif includes a phosphodiester derivative internucleotide linkage.

As used herein, the term “Class A CpG ODN” or “A-class CpG ODN” or “D-type CpG ODN” or “Class A CpG DNA sequence” is used in accordance with its common meaning in the biological and chemical sciences and refers to a CpG motif including oligodeoxynucleotide including one or more of poly-G sequence at the 5′, 3′, or both ends; an internal palindrome sequence including CpG motif, or one or more phosphodiester derivatives linking deoxynucleotides. In embodiments, a Class A CpG ODN includes poly-G sequence at the 5′, 3′, or both ends; an internal palindrome sequence including CpG motif, and one or more phosphodiester derivatives linking deoxynucleotides. In embodiments, the phosphodiester derivative is phosphorothioate.

As used herein, the term “Class B CpG ODN” or “B-class CpG ODN” or “K-type CpG ODN” or “Class B CpG DNA sequence” is used in accordance with its common meaning in the biological and chemical sciences and refers to a CpG motif including oligodeoxynucleotide including one or more of a 6mer motif including a CpG motif, phosphodiester derivatives linking all deoxynucleotides. In embodiments, a Class B CpG ODN includes one or more copies of a 6mer motif including a CpG motif and phosphodiester derivatives linking all deoxynucleotides. In embodiments, the phosphodiester derivative is phosphorothioate. In embodiments, a Class B CpG ODN includes one 6mer motif including a CpG motif. In embodiments, a Class B CpG ODN includes two copies of a 6mer motif including a CpG motif. In embodiments, a Class B CpG ODN includes three copies of a 6mer motif including a CpG motif. In embodiments, a Class B CpG ODN includes four copies of a 6mer motif including a CpG motif.

As used herein, the term “Class C CpG ODN” or “C-class CpG ODN” or “C-type CpG DNA sequence” is used in accordance with its common meaning in the biological and chemical sciences and refers to an oligodeoxynucleotide including a palindrome sequence including a CpG motif and phosphodiester derivatives (phosphorothioate) linking all deoxynucleotides.

The term “NF-κB” and “NF-κB” and “NFKB” refers to a Nuclear Factor Kappa-Light-Chain-Enhancer of Activated B Cells protein and homologs thereof. In embodiments, “NF-κB” refers to the protein associated with Entrez Gene 4790, OMIM 164011, or UniProt P19838. In embodiments, “NF-κB” refers to the human protein. Included in the term “NF-κB” are the wildtype and mutant forms of the protein. In embodiments, “NF-κB” refers to the wildtype protein. In embodiments, “NF-κB” refers to a mutant protein. In embodiments, the reference numbers immediately above refer to the protein, and associated nucleic acids, known as of the date of filing of this application.

As used herein, the term “nucleic acid sequence capable of binding NF-κB” refers to a nucleic acid sequence optionally including one or more spacers within the sequence, wherein at least a portion of the sequence is an “NF-κB-binding site nucleic acid sequence”.

The term “NF-κB-binding site nucleic acid sequence” or “NF-κB-binding substituent” refers to a nucleic acid including one or more nucleic acids capable of binding to a NF-κB.

In embodiments, a NF-κB-binding substituent includes DNA (e.g. including phosphodiester internucleotide linkages, phosphodiester derivative internucleotide linkages, or a combination of phosphodiester and phosphodiester derivative internucleotide linkages). In embodiments, a NF-κB binding substituent includes a DNA sequence identical (except that it may include one or more phosphodiester derivative linkage(s)) to the genomic DNA sequence a NF-κB binds when modulating transcription. In embodiments, a NF-κB-binding substituent is a DNA sequence identical (except that it may include one or more phosphodiester derivative linkage(s)) to the genomic DNA sequence a NF-κB transcription factor binds when modulating transcription. In embodiments, the NF-κB-binding nucleic acid substituent includes at least one nucleic acid analog having an alternate backbone (e.g. phosphodiester derivative (e.g. phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite), peptide nucleic acid backbone(s), LNA, or linkages). In embodiments, an NF-κB-binding nucleic acid substituent includes or is DNA. In embodiments, an NF-κB-binding nucleic acid substituent includes or is RNA. In embodiments, an NF-κB-binding nucleic acid substituent includes or is a nucleic acid having internucleotide linkages selected from phosphodiesters and phosphodiester derivatives (e.g. phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, O-methylphosphoroamidite, or combinations thereof).

As used herein, the term “NF-κB-binding DNA substituent” refers to a substituent or moiety capable of binding to a NF-κB, including at least one deoxyribonucleic acid. In embodiments, a NF-κB-binding DNA substituent is a nucleic acid. In embodiments, the NF-κB-binding DNA substituent includes at least one nucleic acid analog. In embodiments, the NF-κB-binding DNA substituent includes at least one nucleic acid analog having an alternate backbone (e.g. phosphodiester derivative (e.g. phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite), peptide nucleic acid backbone(s), LNA, or linkages). In embodiments, a NF-κB-binding DNA substituent includes DNA. In embodiments, all nucleotide sugars in a NF-κB-binding DNA substituent are deoxyribose (e.g., all nucleotides are DNA). In embodiments, a NF-κB-binding DNA substituent consists of DNA. In embodiments, a NF-κB-binding DNA substituent includes or is DNA having internucleotide linkages selected from phosphodiesters and phosphodiester derivatives (e.g. phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, O-methylphosphoroamidite, or combinations thereof). In embodiments, a NF-κB-binding DNA substituent consists of DNA having internucleotide linkages selected from phosphodiesters and phosphorothioates. In embodiments, a NF-κB-binding DNA substituent includes or is DNA having backbone linkages selected from phosphodiesters and phosphorodithioates. In embodiments, a NF-κB-binding DNA substituent includes or is DNA including phosphodiester backbone linkages. In embodiments, a NF-κB-binding DNA substituent includes or is DNA including phosphorothioate backbone linkages. In embodiments, a NF-κB-binding DNA substituent includes or is DNA including phosphorodithioate backbone linkages. In embodiments, a NF-κB-binding DNA substituent specifically binds a cellular subcompartment (e.g. endosome) associated NF-κB. In embodiments, a NF-κB-binding DNA substituent includes or is a G-rich oligonucleotide (e.g. about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% G nucleotides; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% G nucleotides). In embodiments, a NF-κB-binding DNA substituent includes a CpG motif, wherein C and G are nucleotides and p is the phosphate connecting the C and G. In embodiments, the CpG motif is unmethylated. In embodiments, a NF-κB-binding DNA substituent is a Class A CpG oligodeoxynucleotide (ODN). In embodiments, a NF-κB-binding DNA substituent is a Class B CpG oligodeoxynucleotide (ODN). In embodiments, a NF-κB-binding DNA substituent is a Class C CpG oligodeoxynucleotide (ODN). In embodiments, a NF-κB-binding DNA substituent consists of deoxyribonucleic acids with A, G, C, or T bases and phosphodiester linkages and/or phosphodiester derivative linkages (e.g., phosphorothioate linkage(s)).

As used herein, the term “checkpoint inhibitor” or “checkpoint inhibitor therapy” refers to an inhibitor or therapy, respectively, that target immune checkpoints, key regulators of the immune system that when stimulated can dampen the immune response to an immunologic stimulus. In embodiments, the checkpoint inhibitor is an anti-cancer drug. In embodiments, the checkpoint inhibitor inhibits PD-1, PD-L1, or CTLA-4 (e.g., inhibit level of target or inhibit level of activity of target). In embodiments, the checkpoint inhibitor is pembrolizumab, nivolumab, spartalizumab (PDR001), cemiplimab, AMP-224, AMP-514, PDR001, atezolizumab, avelumab, durvalumab, BMX-936559, CK-301, ipilimumab, or tremelimumab.

As used herein, the term “PD-1”, or “CD279” refers to the protein “programmed cell death protein i”. In embodiments, “PD-1”, or “CD279” refers to the human protein. Included in the term “PD-1”, or “CD279” are the wildtype and mutant forms of the protein. In embodiments, “PD-1”, or “CD279” refers to the protein associated with Entrez Gene 5133, UniProt Q15116, and/or RefSeq (protein) NP_005009. In embodiments, the reference numbers immediately above refer to the protein, and associated nucleic acids, known as of the date of filing of this application. In embodiments, “PD-1”, or “CD279” refers to the wildtype human protein. In embodiments, “PD-1”, or “CD279” refers to the wildtype human nucleic acid. In embodiments, the PD-1 receptor is a mutant PD-1 receptor. In embodiments, the mutant PD-1 receptor is associated with a disease that is not associated with wildtype PD-1 receptor. In embodiments, the PD-1 receptor includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype PD-1 receptor. In embodiments, the PD-1 receptor has the protein sequence corresponding to RefSeq NP_005009.2. In embodiments, the PD-1 receptor has the sequence corresponding to RefSeq NM_005018.3. In embodiments, the PD-1 receptor has the following amino acid sequence:

(SEQ ID NO: 18)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

As used herein, the term “PDL-1”, “PD-L1”, “CD274”, “cluster of differentiation 274”, “B7-H1”, or “B7 homolog 1” refers to the protein “programmed death-ligand 1”. In embodiments, “PDL-1”, “PD-L1”, “CD274”, “cluster of differentiation 274”, “B7-H1”, or “B7 homolog 1” refers to the human protein. Included in the term “PDL-1”, “PD-L1”, “CD274”, “cluster of differentiation 274”, “B7-H1”, or “B7 homolog 1” are the wildtype and mutant forms of the protein. In embodiments, “PDL-1”, “PD-L1”, “CD274”, “cluster of differentiation 274”, “B7-H1”, or “B7 homolog 1” refers to the protein associated with Entrez Gene 29126, UniProt Q9NZQ7, and/or RefSeq (protein) NP_054862. In embodiments, the reference numbers immediately above refer to the protein, and associated nucleic acids, known as of the date of filing of this application. In embodiments, “PDL-1”, “PD-L1”, “CD274”, “cluster of differentiation 274”, “B7-H1”, or “B7 homolog 1” refers to the wildtype human protein. In embodiments, “PDL-1”, “PD-L1”, “CD274”, “cluster of differentiation 274”, “B7-H1”, or “B7 homolog 1” refers to the wildtype human nucleic acid. In embodiments, the PDL-1 protein is a mutant PDL-1 protein. In embodiments, the mutant PDL-1 protein is associated with a disease that is not associated with wildtype PDL-1 protein. In embodiments, the PDL-1 protein includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype PDL-1 protein. In embodiments, PDL-1 has the protein sequence corresponding to RefSeq NP_054862.1. In embodiments, PDL-1 has the sequence corresponding to RefSeq NM_014143.4. In embodiments, the PDL-1 receptor has the following amino acid sequence:

(SEQ ID NO: 19)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET.

As used herein, the term “CTLA-4”, “CTLA4”, “CD152”, or “cluster of differentiation 152” refers to the protein receptor “cytotoxic T-lymphocyte-associated protein 4”. In embodiments, “CTLA-4”, “CTLA4”, “CD152”, or “cluster of differentiation 152” refers to the human protein. Included in the term “CTLA-4”, “CTLA4”, “CD152”, or “cluster of differentiation 152” are the wildtype and mutant forms of the protein. In embodiments, “CTLA-4”, “CTLA4”, “CD152”, or “cluster of differentiation 152” refers to the protein associated with Entrez Gene 1493, UniProt P16410, and/or RefSeq (protein) NP_005205. In embodiments, the reference numbers immediately above refer to the protein, and associated nucleic acids, known as of the date of filing of this application. In embodiments, “CTLA-4”, “CTLA4”, “CD152”, or “cluster of differentiation 152” refers to the wildtype human protein. In embodiments, “CTLA-4”, “CTLA4”, “CD152”, or “cluster of differentiation 152” refers to the wildtype human nucleic acid. In embodiments, the CTLA-4 protein is a mutant PDL-1 protein. In embodiments, the mutant CTLA-4 protein is associated with a disease that is not associated with wildtype CTLA-4 protein. In embodiments, the CTLA-4 protein includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype CTLA-4 protein. In embodiments, the CTLA-4 receptor has the protein sequence corresponding to RefSeq NP_005205.2. In embodiments, the CTLA-4 receptor has the sequence corresponding to RefSeq NM_005214.5. In embodiments, the CTLA-4 receptor has the following amino acid sequence:

(SEQ ID NO: 20)
MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
YVKMPPTEPECEKQFQPYFIPIN.

As used herein, the term “chimeric antigen receptor T cells” or “CAR-T cells” refer to T cells that have been genetically engineered to produce an artificial T-cell receptor for use in immunotherapy. In embodiments, CAR-T cells are autologous, meaning they are derived from T cells in a patient's own blood. In embodiments, CAR-T cells are allogenic, meaning they are derived from the T cells of another healthy donor. CAR-T cells have the ability to bind to a target antigen. In embodiments, the target antigen is CD19.

As used herein, the term “chimeric antigen receptors”, “CARs”, “chimeric immunoreceptors”, “chimeric T cell receptors”, or “artificial T cell receptors” refer to proteins that have been engineered to give T cells the new ability to target a specific protein. The receptors are chimeric because they combine both antigen-binding and T-cell activating functions into a single receptor.

As used herein, the term “Tisagenlecleucel”, “CTL019”, or “Kymriah™” is used in accordance with its well understood meaning and refers to a CAR-T cell that is approved for the treatment of B-cell acute lymphoblastic leukemia (ALL) and relapsed or refractory diffuse large B-cell lymphoma.

As used herein, the term “Axicabtagene ciloleucel”, “KTE-C19”, “Axi-cel”, or “Yescarta®” is used in accordance with its well understood meaning and refers to a CAR-T cell that is approved for the treatment of B-cell lymphomas, including diffuse large B-cell lymphoma, transformed follicular lymphoma, and primary mediastinal B-cell lymphoma.

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), van der waal's bond(s)/interactions, hydrogen bond(s), polar bond(s), or combinations or mixtures thereof).

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

The term “capable of binding” as used herein refers to a moiety (e.g. a compound as described herein) that is able to measurably bind to a target (e.g., a NF-κB, a Toll-like receptor protein). In embodiments, where a moiety is capable of binding a target, the moiety is capable of binding with a Kd of less than about 10 pM, 5 pM, 1 pM, 500 nM, 250 nM, 100 nM, 75 nM, 50 nM, 25 nM, 15 nM, 10 nM, 5 nM, 1 nM, or about 0.1 nM. In embodiments, where a moiety is capable of binding a target, the moiety is capable of binding with a Kd of less than about 10 pM. In embodiments, where a moiety is capable of binding a target, the moiety is capable of binding with a Kd of less than about 5 pM. In embodiments, where a moiety is capable of binding a target, the moiety is capable of binding with a Kd of less than about 1 pM. In embodiments, where a moiety is capable of binding a target, the moiety is capable of binding with a Kd of less than about 500 nM. In embodiments, where a moiety is capable of binding a target, the moiety is capable of binding with a Kd of less than about 250 nM. In embodiments, where a moiety is capable of binding a target, the moiety is capable of binding with a Kd of less than about 100 nM. In embodiments, where a moiety is capable of binding a target, the moiety is capable of binding with a Kd of less than about 75 nM. In embodiments, where a moiety is capable of binding a target, the moiety is capable of binding with a Kd of less than about 50 nM. In embodiments, where a moiety is capable of binding a target, the moiety is capable of binding with a Kd of less than about 25 nM. In embodiments, where a moiety is capable of binding a target, the moiety is capable of binding with a Kd of less than about 15 nM. In embodiments, where a moiety is capable of binding a target, the moiety is capable of binding with a Kd of less than about 10 nM. In embodiments, where a moiety is capable of binding a target, the moiety is capable of binding with a Kd of less than about 5 nM. In embodiments, where a moiety is capable of binding a target, the moiety is capable of binding with a Kd of less than about 1 nM. In embodiments, where a moiety is capable of binding a target, the moiety is capable of binding with a Kd of less than about 0.1 nM.

A “polyglycol” as used herein refers to a poly alkyl ether. In embodiments the poly glycol is substituted (e.g., e.g., substituted with a substituent group, a size-limited substituent group, or lower substituent group). In embodiments, the polyglycol has the formula

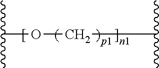

wherein p1 is an integer from 1 to 20 (e.g., 1, 2, 3, 4, 5, 6, 7, 8, 9, 10, 11, 12, 13, 14, 15, 16, 17, 18, 19, or 20); n1 is an integer from 1 to 100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100). In embodiments, wherein p1 is 2, the polyglycol may be referred to as polyethylene glycol. In embodiments, wherein p1 is 3, the polyglycol may be referred to as polypropylene glycol. In embodiments, a polyglycol spacer has the formula:

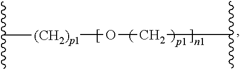

wherein p1 and n1 are described herein; when the polyglycol spacer connects two nucleic acid sequences, through the 3′ terminus of one nucleic acid sequence and the 5′ terminus of the second nucleic acid sequence, wherein one nucleic acid sequence includes a 3′ terminal phosphodiester or phosphodiester derivative, and the second nucleic acid sequence includes a 5′ terminal phosphodiester or phosphodiester derivative. In embodiments, p1 is

The terms “monophosphate” is used in accordance with its ordinary meaning in the arts and refers to a moiety having the formula:

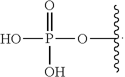 [SMILES: *OP(=O)(O)O]

The term “polyphosphate” refers to at least two phosphate groups, having the formula:

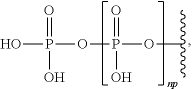

wherein np is an integer of 1 or greater. In embodiments, np is an integer from 0 to 5. In embodiments, np is an integer from 0 to 2. In embodiments, np is 2.

A terminal moiety is a chemically reactive moiety, detectable moiety, therapeutic moiety (e.g. anti-cancer agent or anti-viral agent), nucleic acid sequence, DNA sequence, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, a terminal moiety is a chemically reactive moiety, detectable moiety, therapeutic moiety (e.g. anti-cancer agent or anti-viral agent), nucleic acid sequence, DNA sequence, nucleic acid analogs, R¹-substituted or unsubstituted alkyl, R¹-substituted or unsubstituted heteroalkyl, R¹-substituted or unsubstituted cycloalkyl, R¹-substituted or unsubstituted heterocycloalkyl, R¹-substituted or unsubstituted aryl, or R¹-substituted or unsubstituted heteroaryl. In embodiments, a terminal moiety is a detectable moiety. In embodiments, the detectable moiety is a fluorescent dye, electron-dense reagent, enzyme, biotin, digoxigenin, paramagnetic molecule, paramagnetic nanoparticle, contrast agent, magnetic resonance contrast agent, X-ray contrast agent, Gadolinium, radioisotope, radionuclide, fluorodeoxyglucose, gamma ray emitting radionuclide, positron-emitting radionuclide, biocolloid, microbubble, iodinated contrast agent, barium sulfate, thorium dioxide, gold, gold nanoparticle, gold nanoparticle aggregate, fluorophore, two-photon fluorophore, hapten, protein, or fluorescent moiety. In embodiments, a terminal moiety is a therapeutic moiety (e.g. anti-cancer agent or anti-viral agent).

The term “infection” or “infectious disease” refers to a disease or condition that can be caused by organisms such as a bacterium, virus, fungi or any other pathogenic microbial agents. In embodiments, the infectious disease is caused by a pathogenic bacteria. Pathogenic bacteria are bacteria which cause diseases (e.g., in humans). In embodiments, the infectious disease is a bacteria associated disease (e.g., tuberculosis, which is caused by Mycobacterium tuberculosis). Non-limiting bacteria associated diseases include pneumonia, which may be caused by bacteria such as Streptococcus and Pseudomonas; or foodborne illnesses, which can be caused by bacteria such as Shigella, Campylobacter, and Salmonella. Bacteria associated diseases also includes tetanus, typhoid fever, diphtheria, syphilis, and leprosy. In embodiments, the disease is Bacterial vaginosis (i.e. bacteria that change the vaginal microbiota caused by an overgrowth of bacteria that crowd out the Lactobacilli species that maintain healthy vaginal microbial populations) (e.g., yeast infection, or Trichomonas vaginalis); Bacterial meningitis (i.e. a bacterial inflammation of the meninges); Bacterial pneumonia (i.e. a bacterial infection of the lungs); Urinary tract infection; Bacterial gastroenteritis; or Bacterial skin infections (e.g. impetigo, or cellulitis). In embodiments, the infectious disease is a Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, or Vibrio cholera infection.

The terms “graft-versus-host disease” and “GVHD” are used in accordance with its plain ordinary meaning and refers to a reaction of donor immune cells against host tissues following a tissue transplant. GVHD may be characterized by selective damage to the liver, skin, mucosa, or the gastrointestinal tract. GVHD is a clinical diagnosis that may be supported with appropriate biopsies. In embodiments, GVHD is caused by having white blood cells present within the transplanted tissue attack the recipient's body's cells. In embodiments, GVHD is not equivalent to a transplant rejection, which is understood to occur when the immune system of the transplant recipient rejects the transplanted tissue; GVHD occurs when the donor's immune system's white blood cells reject the recipient. Acute GVHD (aGVHD) is used in accordance with its ordinary meaning in the arts and refers to an onset of GVHD within about 100 days following a tissue transplant. Additional information about GVHD and aGVHD may be found in Jacobsohn et al (Jacobsohn, D. A., & Vogelsang, G. B. (2007). Acute graft versus host disease. Orphanet Journal of Rare Diseases, 2, 35. http://doi.org/10.1186/1750-1172-2-35), which is incorporated herein by reference.

The term “allotransplantation” refers to the transplantation of cells, tissues, or organs, to a recipient from a genetically non-identical donor of the same species.

The terms “allogeneic Hematopoietic cell transplantation” and “allo-HCT” refers to a medical procedure in which a person receives blood-forming stem cells from a genetically similar, though not necessarily identical, donor. This is often a sister or brother, but could be an unrelated donor.

### II. Compounds

In an aspect is provided a compound including a first nucleic acid sequence capable of binding to Nuclear Factor Kappa-Light-Chain-Enhancer of Activated B Cells (NF-κB) and a second nucleic acid sequence capable of binding a Toll-like receptor (TLR) protein, wherein the first nucleic acid sequence is covalently bound to the second nucleic acid sequence.

In embodiments, the first nucleic acid sequence capable of binding to NF-κB includes a first NF-κB binding site nucleic acid sequence and a second NF-κB binding site nucleic acid sequence that are connected through a first spacer. In embodiments, the first spacer is a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, the first spacer is substituted (e.g., substituted with a substituent group, a size-limited substituent group, or lower substituent group) or unsubstituted polyglycol, substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, where the first spacer is substituted, it is substituted with a substituent group. In embodiments, where the first spacer is substituted, it is substituted with a size-limited substituent group. In embodiments, where the first spacer is substituted, it is substituted with a lower substituent group.

In embodiments, the first spacer is unsubstituted polyglycol, unsubstituted alkylene, unsubstituted heteroalkylene, unsubstituted cycloalkylene, unsubstituted heterocycloalkylene, unsubstituted arylene, or unsubstituted heteroarylene.

In embodiments, the second nucleic acid sequence capable of binding a TLR protein includes a first TLR binding site nucleic acid sequence and a second TLR site nucleic acid sequence connected through a second spacer. In embodiments, the second spacer is a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, the compound includes a plurality of spacers (e.g., first spacer, second spacer, covalent spacer, third spacer, fourth spacer, aliphatic spacer, additional spacers). In embodiments, wherein a plurality of spacers are present, it is understood that each spacer is independent (e.g. optionally different).

In embodiments, the second spacer is substituted (e.g., substituted with a substituent group, a size-limited substituent group, or lower substituent group) or unsubstituted polyglycol, substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, where the second spacer is substituted, it is substituted with a substituent group. In embodiments, where the second spacer is substituted, it is substituted with a size-limited substituent group. In embodiments, where the second spacer is substituted, it is substituted with a lower substituent group.

In embodiments, the second spacer is unsubstituted polyglycol, unsubstituted alkylene, unsubstituted heteroalkylene, unsubstituted cycloalkylene, unsubstituted heterocycloalkylene, unsubstituted arylene, or unsubstituted heteroarylene.

In an aspect is provided a compound including a first nucleic acid sequence capable of binding to Nuclear Factor Kappa-Light-Chain-Enhancer of Activated B Cells (NF-κB) and a second nucleic acid sequence capable of binding a Toll-like receptor (TLR) protein, wherein said first nucleic acid sequence and said second nucleic acid sequence are covalently bound through a covalent spacer, wherein the covalent spacer is a bond, a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, the covalent spacer is a bond. In embodiments, the covalent spacer is a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, the covalent spacer is substituted (e.g., substituted with a substituent group, a size-limited substituent group, or lower substituent group) or unsubstituted polyglycol, substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, where the covalent spacer is substituted, it is substituted with a substituent group. In embodiments, where the covalent spacer is substituted, it is substituted with a size-limited substituent group. In embodiments, where the covalent spacer is substituted, it is substituted with a lower substituent group.

In embodiments, the covalent spacer is unsubstituted polyglycol, unsubstituted alkylene, unsubstituted heteroalkylene, unsubstituted cycloalkylene, unsubstituted heterocycloalkylene, unsubstituted arylene, or unsubstituted heteroarylene.

In embodiments, the compound includes a first terminal moiety that is covalently bound through a third spacer to the first nucleic acid sequence.

In embodiments, the compound includes a second terminal moiety that is covalently bound through a fourth spacer to the second nucleic acid sequence.

In embodiments, the compound includes a first terminal moiety that is covalently bound through a third spacer to the first nucleic acid sequence, and a second terminal moiety that is covalently bound through a fourth spacer to the second nucleic acid sequence.

In embodiments, the third spacer is a bond, substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, the third spacer is a bond. In embodiments, the third spacer is a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, the third spacer is substituted (e.g., substituted with a substituent group, a size-limited substituent group, or lower substituent group) or unsubstituted polyglycol, substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, where the third spacer is substituted, it is substituted with a substituent group. In embodiments, where the third spacer is substituted, it is substituted with a size-limited substituent group. In embodiments, where the third spacer is substituted, it is substituted with a lower substituent group.

In embodiments, the third spacer is unsubstituted polyglycol, unsubstituted alkylene, unsubstituted heteroalkylene, unsubstituted cycloalkylene, unsubstituted heterocycloalkylene, unsubstituted arylene, or unsubstituted heteroarylene.

In embodiments, the fourth spacer is a bond, substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, the fourth spacer is a bond. In embodiments, the fourth spacer is a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, the fourth spacer is substituted (e.g., substituted with a substituent group, a size-limited substituent group, or lower substituent group) or unsubstituted polyglycol, substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, where the fourth spacer is substituted, it is substituted with a substituent group. In embodiments, where the fourth spacer is substituted, it is substituted with a size-limited substituent group. In embodiments, where the fourth spacer is substituted, it is substituted with a lower substituent group.

In embodiments, the fourth spacer is unsubstituted polyglycol, unsubstituted alkylene, unsubstituted heteroalkylene, unsubstituted cycloalkylene, unsubstituted heterocycloalkylene, unsubstituted arylene, or unsubstituted heteroarylene.

In embodiments, the first spacer is a substituted or unsubstituted C₁-C₄₀ alkylene, substituted or unsubstituted 2 to 40 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

In embodiments, the second spacer is a substituted or unsubstituted C₁-C₄₀ alkylene, substituted or unsubstituted 2 to 40 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

In embodiments, the covalent spacer is a substituted or unsubstituted C₁-C₄₀ alkylene, substituted or unsubstituted 2 to 40 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

In embodiments, the third spacer is a substituted or unsubstituted C₁-C₄₀ alkylene, substituted or unsubstituted 2 to 40 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

In embodiments, the fourth spacer is a substituted or unsubstituted C₁-C₄₀ alkylene, substituted or unsubstituted 2 to 40 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

The first terminal moiety is hydrogen, chemically reactive moiety, detectable moiety, therapeutic moiety (e.g. anti-cancer agent or anti-viral agent), a nucleic acid sequence, DNA sequence, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the first terminal moiety is a chemically reactive moiety, detectable moiety, therapeutic moiety (e.g. anti-cancer agent or anti-viral agent), nucleic acid sequence, DNA sequence, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the first terminal moiety is a detectable moiety. In embodiments, the detectable moiety is a fluorescent dye, electron-dense reagent, enzyme, biotin, digoxigenin, paramagnetic molecule, paramagnetic nanoparticle, contrast agent, magnetic resonance contrast agent, X-ray contrast agent, Gadolinium, radioisotope, radionuclide, fluorodeoxyglucose, gamma ray emitting radionuclide, positron-emitting radionuclide, biocolloid, microbubble, iodinated contrast agent, barium sulfate, thorium dioxide, gold, gold nanoparticle, gold nanoparticle aggregate, fluorophore, two-photon fluorophore, hapten, protein, or fluorescent moiety. In embodiments, the first terminal moiety is a therapeutic moiety (e.g. anti-cancer agent or anti-viral agent).

The second terminal moiety is hydrogen, chemically reactive moiety, detectable moiety, therapeutic moiety (e.g. anti-cancer agent or anti-viral agent), a nucleic acid sequence, DNA sequence, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the second terminal moiety is a chemically reactive moiety, detectable moiety, therapeutic moiety (e.g. anti-cancer agent or anti-viral agent), nucleic acid sequence, DNA sequence, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the second terminal moiety is a detectable moiety. In embodiments, the detectable moiety is a fluorescent dye, electron-dense reagent, enzyme, biotin, digoxigenin, paramagnetic molecule, paramagnetic nanoparticle, contrast agent, magnetic resonance contrast agent, X-ray contrast agent, Gadolinium, radioisotope, radionuclide, fluorodeoxyglucose, gamma ray emitting radionuclide, positron-emitting radionuclide, biocolloid, microbubble, iodinated contrast agent, barium sulfate, thorium dioxide, gold, gold nanoparticle, gold nanoparticle aggregate, fluorophore, two-photon fluorophore, hapten, protein, or fluorescent moiety. In embodiments, the second terminal moiety is a therapeutic moiety (e.g. anti-cancer agent or anti-viral agent).

A spacer (e.g., first spacer, second spacer, covalent spacer, third spacer, or fourth spacer) is independently a bond, nucleic acid sequence, two nucleic acid sequences, DNA sequence, two DNA sequences, nucleic acid analog sequence, substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. The first spacer, second spacer, covalent spacer, third spacer, and fourth spacer are independently a bond, nucleic acid sequence, two nucleic acid sequences, DNA sequence, two DNA sequences, nucleic acid analog sequence, substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

It will be understood by a person having ordinary skill in the art that in embodiments, when the first spacer is a bond, the first NF-κB binding site nucleic acid sequence and second NF-κB binding site nucleic acid sequence that are connected through the first spacer are bonded through a single phosphodiester or phosphodiester derivative between the terminal 3′ carbon of one NF-κB binding site nucleic acid sequence and the terminal 5′ carbon of the other NF-κB binding site nucleic acid sequence.

It will be understood by a person having ordinary skill in the art that in embodiments, when the second spacer is a bond, the first TLR binding site nucleic acid sequence and second TLR site nucleic acid sequence that are connected through the second spacer are bonded through a single phosphodiester or phosphodiester derivative between the terminal 3′ carbon of one TLR binding site nucleic acid sequence and the terminal 5′ carbon of the other TLR binding site nucleic acid sequence.

It will be understood by a person having ordinary skill in the art that in embodiments, when the covalent spacer is a bond, the first nucleic acid sequence capable of binding to Nuclear Factor Kappa-Light-Chain-Enhancer of Activated B Cells (NF-κB) and the second nucleic acid sequence capable of binding a Toll-like receptor (TLR) protein that are connected through the covalent spacer are bonded through a single phosphodiester or phosphodiester derivative between the terminal 3′ carbon of one nucleic acid sequence (e.g., first nucleic acid sequence capable of binding to Nuclear Factor Kappa-Light-Chain-Enhancer of Activated B Cells (NF-κB) or second nucleic acid sequence capable of binding a Toll-like receptor (TLR) protein) and the terminal 5′ carbon of the other nucleic acid sequence (e.g., second nucleic acid sequence capable of binding a Toll-like receptor (TLR) protein or first nucleic acid sequence capable of binding to Nuclear Factor Kappa-Light-Chain-Enhancer of Activated B Cells (NF-κB)).

In embodiments, the third spacer and first terminal moiety are hydrogen and the compound includes a 5′ terminal OH group bonded to the 5′ terminal carbon of the compound.

In embodiments, the third spacer and first terminal moiety are hydrogen and the compound includes a 3′ terminal OH group bonded to the 3′ terminal carbon of the compound.

In embodiments, the fourth spacer and second terminal moiety are hydrogen and the compound includes a 5′ terminal OH group bonded to the 5′ terminal carbon of the compound.

In embodiments, the fourth spacer and second terminal moiety are hydrogen and the compound includes a 3′ terminal OH group bonded to the 3′ terminal carbon of the compound.

In embodiments, the compound includes an OH group bonded to the 5′ carbon of the 5′ terminal nucleotide and the compound does not include a third spacer and/or first terminal group. In embodiments, the compound includes an OH group bonded to the 3′ carbon of the 3′ terminal nucleotide and the compound does not include a third spacer and/or first terminal group.

In embodiments, the compound includes an OH group bonded to the 5′ carbon of the 5′ terminal nucleotide and the compound does not include a fourth spacer and/or second terminal group. In embodiments, the compound includes an OH group bonded to the 3′ carbon of the 3′ terminal nucleotide and the compound does not include a fourth spacer and/or second terminal group.

In embodiments, the third spacer and first terminal moiety are hydrogen and the compound includes a 5′ terminal phosphate or phosphate derivative group bonded to the 5′ terminal carbon of the compound.

In embodiments, the third spacer and first terminal moiety are hydrogen and the compound includes a 3′ terminal phosphate or phosphate derivative group bonded to the 3′ terminal carbon of the compound.

In embodiments, the fourth spacer and second terminal moiety are hydrogen and the compound includes a 5′ terminal phosphate or phosphate derivative group bonded to the 5′ terminal carbon of the compound.

In embodiments, the fourth spacer and second terminal moiety are hydrogen and the compound includes a 3′ terminal phosphate or phosphate derivative group bonded to the 3′ terminal carbon of the compound.

In embodiments, the compound includes a phosphate or phosphate derivative group bonded to the 5′ carbon of the 5′ terminal nucleotide and the compound does not include a third spacer and/or first terminal group. In embodiments, the compound includes a phosphate or phosphate derivative group bonded to the 3′ carbon of the 3′ terminal nucleotide and the compound does not include a third spacer and/or first terminal group.

In embodiments, the compound includes a phosphate or phosphate derivative group bonded to the 5′ carbon of the 5′ terminal nucleotide and the compound does not include a fourth spacer and/or second terminal group. In embodiments, the compound includes a phosphate or phosphate derivative group bonded to the 3′ carbon of the 3′ terminal nucleotide and the compound does not include a fourth spacer and/or second terminal group.

In embodiments, a spacer (e.g., first spacer, second spacer, covalent spacer, third spacer, or fourth spacer) is independently substituted (e.g., substituted with a substituent group, a size-limited substituent group, or lower substituent group) or unsubstituted polyglycol, substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, where the spacer is substituted, it is substituted with a substituent group. In embodiments, where the spacer is substituted, it is substituted with a size-limited substituent group. In embodiments, where the spacer is substituted, it is substituted with a lower substituent group.

In embodiments, a spacer (e.g., first spacer, second spacer, covalent spacer, third spacer, or fourth spacer) is independently unsubstituted polyglycol, unsubstituted alkylene, unsubstituted heteroalkylene, unsubstituted cycloalkylene, unsubstituted heterocycloalkylene, unsubstituted arylene, or unsubstituted heteroarylene.

In embodiments, the spacer (e.g., first spacer, second spacer, covalent spacer, third spacer, or fourth spacer) is independently a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, the spacer (e.g., first spacer, second spacer, covalent spacer, third spacer, or fourth spacer) is independently a substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, the spacer (e.g., first spacer, second spacer, covalent spacer, third spacer, or fourth spacer) is independently an unsubstituted C₁-C₂₀ alkylene, unsubstituted 2 to 20 membered heteroalkylene, unsubstituted C₃-C₈ cycloalkylene, unsubstituted 3 to 8 membered heterocycloalkylene, unsubstituted C₆-C₁₀ arylene, or unsubstituted 5 to 10 membered heteroarylene. In embodiments, the spacer (e.g., first spacer, second spacer, covalent spacer, third spacer, or fourth spacer) is independently an unsubstituted C₁-C₂₀ alkylene. In embodiments, the spacer (e.g., first spacer, second spacer, covalent spacer, third spacer, or fourth spacer) is independently a substituted or unsubstituted C₁-C₄₀ alkylene, substituted or unsubstituted 2 to 40 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, the spacer (e.g., first spacer, second spacer, covalent spacer, third spacer, or fourth spacer) is independently a substituted or unsubstituted C₁-C₄₀ alkylene. In embodiments, the spacer (e.g., first spacer, second spacer, covalent spacer, third spacer, or fourth spacer) is independently a substituted or unsubstituted 2 to 40 membered heteroalkylene. In embodiments, the spacer (e.g., first spacer, second spacer, covalent spacer, third spacer, or fourth spacer) is a substituted 2 to 40 membered heteroalkylene. In embodiments, the spacer (e.g., first spacer, second spacer, covalent spacer, third spacer, or fourth spacer) includes alkyl phosphates (e.g., propyl phosphates). In embodiments, the spacer (e.g., first spacer, second spacer, covalent spacer, third spacer, or fourth spacer) consists of alkyl phosphates (e.g., propyl phosphates) bonded to the remainder of the compound by phosphates at both ends. In embodiments, the spacer (e.g., first spacer, second spacer, covalent spacer, third spacer, or fourth spacer) consists of 1-5 alkyl phosphates (e.g., propyl phosphates) bonded to the remainder of the compound by phosphates at both ends. In embodiments, the spacer (e.g., first spacer, second spacer, covalent spacer, third spacer, or fourth spacer) consists of 1-4 alkyl phosphates (e.g., propyl phosphates) bonded to the remainder of the compound by phosphates at both ends. In embodiments, the spacer (e.g., first spacer, second spacer, covalent spacer, third spacer, or fourth spacer) consists of 4 alkyl phosphates (e.g., propyl phosphates) bonded to the remainder of the compound by phosphates at both ends. A person having ordinary skill in the art will recognize that a spacer consisting of alkyl phosphates that is bonded to the remainder of the compound by phosphates on both ends will have one more phosphate than alkylene groups (e.g., a spacer (e.g., first spacer, second spacer, covalent spacer, third spacer, or fourth spacer) consisting of 4 alkyl phosphates that is bonded to the remainder of the compound by phosphates at both ends will have five phosphates and four alkyl groups with alternating phosphate groups and alkyl groups).

In embodiments, the spacer (e.g., first spacer, second spacer, or aliphatic spacer) has the formula:

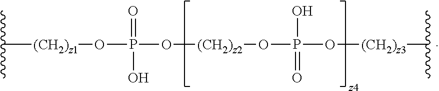

The symbols z1, z2, z3 and z4 are independently integers from 0 to 20.

Compounds as described herein, when they include nucleic acid sequences that are connected by a spacer (e.g., first spacer, second spacer, covalent spacer, third spacer, or fourth spacer), independently include a spacer that is covalently bound to a nucleic acid at the 3′ end, the 5′ end or both through a single phosphodiester group or phosphodiester derivative (e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite), wherein the phosphodiester group or phosphodiester derivative may be provided during the synthesis of the compound by the reactant nucleic acid component or the reactant spacer component.

In embodiments, the spacer (e.g., first spacer, second spacer or aliphatic spacer) has the formula:

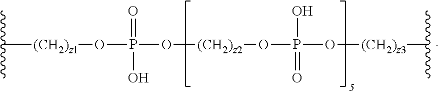

The symbols z1, z2, and z3 are independently integers from 0 to 20.

In embodiments, the spacer (e.g., first spacer, second spacer, or aliphatic spacer) has the formula:

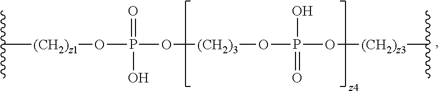

wherein z1, z4, and z3 are as described herein.

In embodiments, the spacer (e.g., first spacer, second spacer, or aliphatic spacer) has the formula:

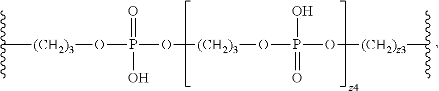

wherein z4 and z3 are as described herein.

In embodiments, the spacer (e.g., first spacer, second spacer or aliphatic spacer) has the formula:

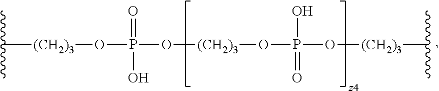

wherein z4 is as described herein.

In embodiments, the spacer (e.g., first spacer, second spacer, or aliphatic spacer) has the formula:

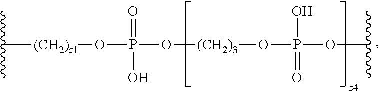

wherein z1 and z4 are as described herein.

In embodiments, the spacer (e.g., first spacer, second spacer, or aliphatic spacer) has the formula:

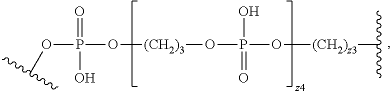

wherein z4 and z3 are as described herein.

In embodiments, the spacer (e.g., first spacer, second spacer, or aliphatic spacer) has the formula:

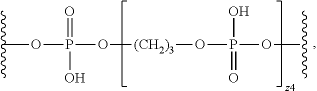

wherein z4 is as described herein.

In embodiments, the covalent spacer has the formula:

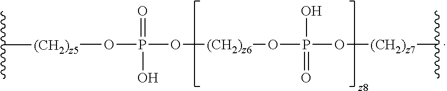

The symbols z5, z6, z7 and z8 are independently integers from 0 to 20.

In embodiments, the covalent spacer has the formula:

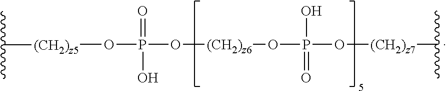

The symbols z5, z6, and z7 are independently integers from 0 to 20.

In embodiments, the covalent spacer has the formula:

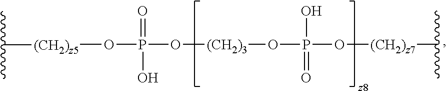

wherein z5, z7, and z8 are as described herein.

In embodiments, the covalent spacer has the formula:

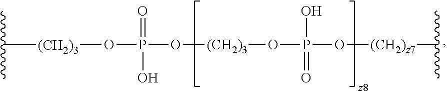

wherein z7 and z8 are as described herein.

In embodiments, the covalent spacer has the formula:

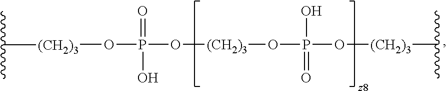

wherein z8 is as described herein.

In embodiments, the covalent spacer has the formula:

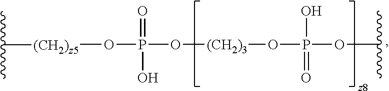

wherein z5 and z8 are as described herein.

In embodiments, the covalent spacer has the formula:

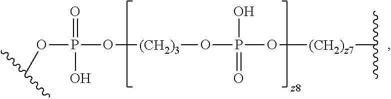

wherein z7 and z8 are as described herein.

In embodiments, the covalent spacer has the formula:

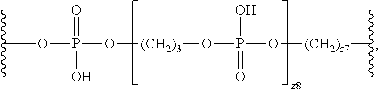

wherein z8 is as described herein.

In embodiments, the third spacer has the formula:

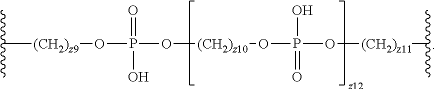

The symbols z9, z10, z11 and z12 are independently integers from 0 to 20.

In embodiments, the third spacer has the formula:

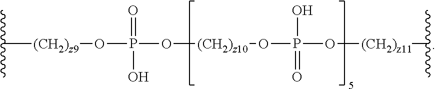

The symbols z9, z10, and z11 are independently integers from 0 to 20.

In embodiments, the third spacer has the formula:

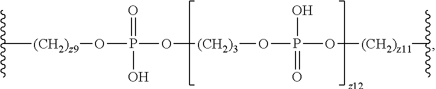

wherein z9, z11, and z12 are as described herein.

In embodiments, the third spacer has the formula:

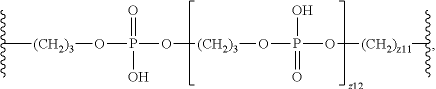

wherein z11 and z12 are as described herein.

In embodiments, the third spacer has the formula:

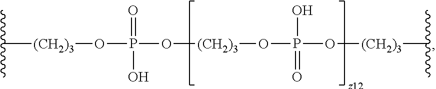

wherein z12 is as described herein.

In embodiments, the third spacer has the formula:

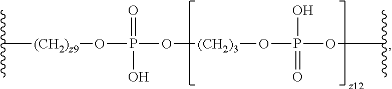

wherein z9 and z12 are as described herein.

In embodiments, the third spacer has the formula:

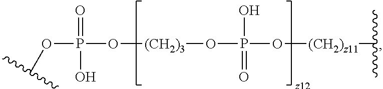

wherein z11 and z12 are as described herein.

In embodiments, the third spacer has the formula:

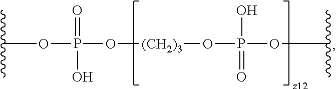

wherein z12 is as described herein.

In embodiments, the fourth spacer has the formula:

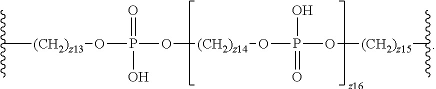

The symbols z13, z14, z15 and z16 are independently integers from 0 to 20.

In embodiments, the fourth spacer has the formula:

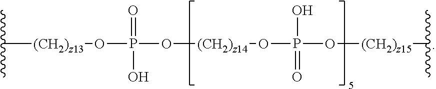

The symbols z13, z14, and z15 are independently integers from 0 to 20.

In embodiments, the fourth spacer has the formula:

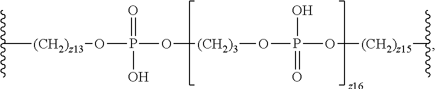

wherein z13, z15, and z16 are as described herein.

In embodiments, the fourth spacer has the formula:

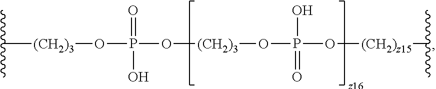

wherein z15 and z16 are as described herein.

In embodiments, the fourth spacer has the formula:

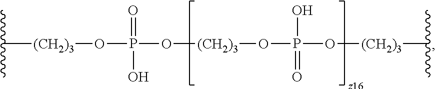

wherein z16 is as described herein.

In embodiments, the fourth spacer has the formula:

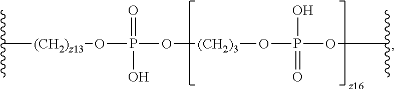

wherein z13 and z16 are as described herein.

In embodiments, the fourth spacer has the formula:

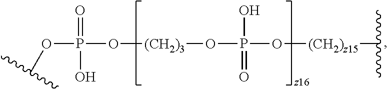

wherein z15 and z16 are as described herein.

In embodiments, the fourth spacer has the formula:

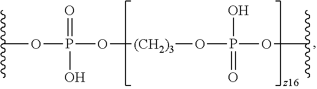

wherein z16 is as described herein.

In embodiments, a nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence) includes a monovalent or divalent phosphate or phosphate derivative bonded to the terminal 5′ carbon of the nucleic acid, the terminal 3′ carbon of the nucleic acid or both the terminal 5′ carbon and terminal 3′ carbon of the nucleic acid. A phosphate group may form a phosphodiester covalently connecting two separate moieties such as an internucleotide linkage between two nucleotides in a nucleic acid. A phosphate derivative may form a phosphodiester derivative covalently connecting two separate moieties such as an internucleotide linkage between two nucleotides in a nucleic acid.

In embodiments, a spacer (e.g., first spacer, second spacer, covalent spacer, third spacer, or fourth spacer) covalently bound to a nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence) is covalently bonded to a phosphodiester or phosphodiester derivative of the nucleic acid sequence, wherein the phosphodiester or phosphodiester derivative is bonded to the terminal 5′ carbon of the nucleic acid sequence.

In embodiments, a spacer (e.g., first spacer, second spacer, covalent spacer, third spacer, or fourth spacer) covalently bound to a nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence) is covalently bonded to a phosphodiester or phosphodiester derivative of the nucleic acid sequence, wherein the phosphodiester or phosphodiester derivative is bonded to the terminal 3′ carbon of the nucleic acid sequence.

In embodiments, the oxygen of the 5′ OH group of a 5′ terminal residue in a nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence) is directly bonded to a phosphorus atom in a phosphate group or phosphate derivative group; or phosphodiester or phosphodiester derivative, that is optionally further bonded to a spacer (e.g., first spacer, second spacer, covalent spacer, third spacer, or fourth spacer) as described herein.

In embodiments, the oxygen of the 3′ OH group of a 3′ terminal residue in a nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence) is directly bonded to a phosphorus atom in a phosphate group or phosphate derivative group; or phosphodiester or phosphodiester derivative, that is optionally further bonded to a spacer (e.g., first spacer, second spacer, covalent spacer, third spacer, or fourth spacer) as described herein.

In embodiments, when a nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence) is directly bonded to a spacer (e.g., first spacer, second spacer, covalent spacer, third spacer, or fourth spacer) at the 5′ terminus of the nucleic acid sequence, the 5′ OH group of the 5′ terminal residue is included in a phosphodiester or phosphodiester derivative covalently bonded to the spacer.

In embodiments, when a nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence) is directly bonded to a spacer (e.g., first spacer, second spacer, covalent spacer, third spacer, or fourth spacer) at the 3′ terminus of the nucleic acid sequence, the 3′ OH group of the 3′ terminal residue is included in a phosphodiester or phosphodiester derivative covalently bonded to the spacer.

In embodiments, when a nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence) is directly bonded to a spacer (e.g., first spacer, second spacer, covalent spacer, third spacer, or fourth spacer) at the 5′ terminus of the nucleic acid sequence, the 5′ phosphodiester or phosphodiester derivative of the 5′ terminal residue is covalently bonded to the spacer.

In embodiments, when a nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence) is directly bonded to a spacer (e.g., first spacer, second spacer, covalent spacer, third spacer, or fourth spacer) at the 3′ terminus of the nucleic acid sequence, the 3′ phosphodiester or phosphodiester derivative of the 3′ terminal residue is covalently bonded to the spacer.

In embodiments, when a spacer (e.g., first spacer, second spacer or aliphatic spacer) is bonded to a terminal 5′ phosphodiester or phosphodiester derivative of one nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence) and a terminal 3′ phosphodiester or phosphodiester derivative of a second nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence), the spacer has the formula:

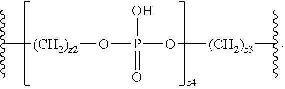

The symbols z2, z3 and z4 are as described herein. In embodiments of the spacer formula immediately above, z3 is not zero.

In embodiments, when a covalent spacer is bonded to a terminal 5′ phosphodiester or phosphodiester derivative of one nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence) and a terminal 3′ phosphodiester or phosphodiester derivative of a second nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence), the spacer has the formula:

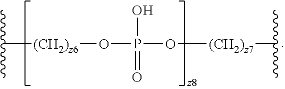

The symbols z6, z7 and z8 are as described herein. In embodiments of the spacer formula immediately above, z7 is not zero.

In embodiments, when a third spacer is bonded to a terminal 5′ phosphodiester or phosphodiester derivative of a nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence), the spacer has the formula:

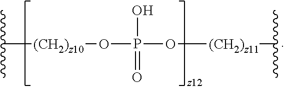

The symbols z10, z11 and z12 are as described herein. In embodiments of the spacer formula immediately above, z11 is not zero.

In embodiments, when a fourth spacer is bonded to a terminal 5′ phosphodiester or phosphodiester derivative of a nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence), the spacer has the formula:

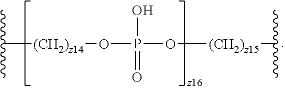

The symbols z14, z15 and z16 are as described herein. In embodiments of the spacer formula immediately above, z15 is not zero.

In embodiments, when a third spacer is bonded to a terminal 3′ phosphodiester or phosphodiester derivative of a nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence), the spacer has the formula:

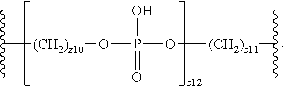

The symbols z10, z11 and z12 are as described herein. In embodiments of the spacer formula immediately above, z11 is not zero.

In embodiments, when a fourth spacer is bonded to a terminal 3′ phosphodiester or phosphodiester derivative of a nucleic acid sequence (e.g., first nucleic acid sequence capable of binding to NF-κB, second nucleic acid sequence capable of binding a TLR protein, first NF-κB binding site nucleic acid sequence, second NF-κB binding site nucleic acid sequence, first TLR binding site nucleic acid sequence, or second TLR site nucleic acid sequence), the spacer has the formula:

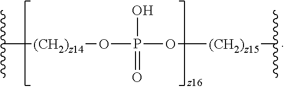

The symbols z14, z15 and z16 are as described herein. In embodiments of the spacer formula immediately above, z15 is not zero.

An example of a spacer, between two nucleotides or two nucleotide sequences, is shown below.

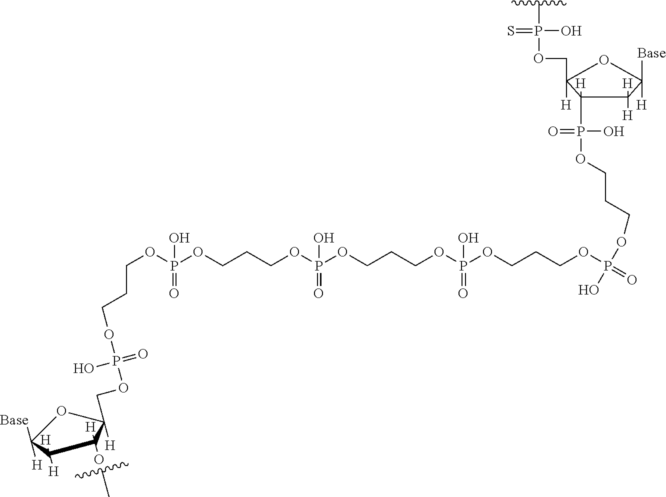

An example of a compound including spacers and a terminal moiety is shown below:

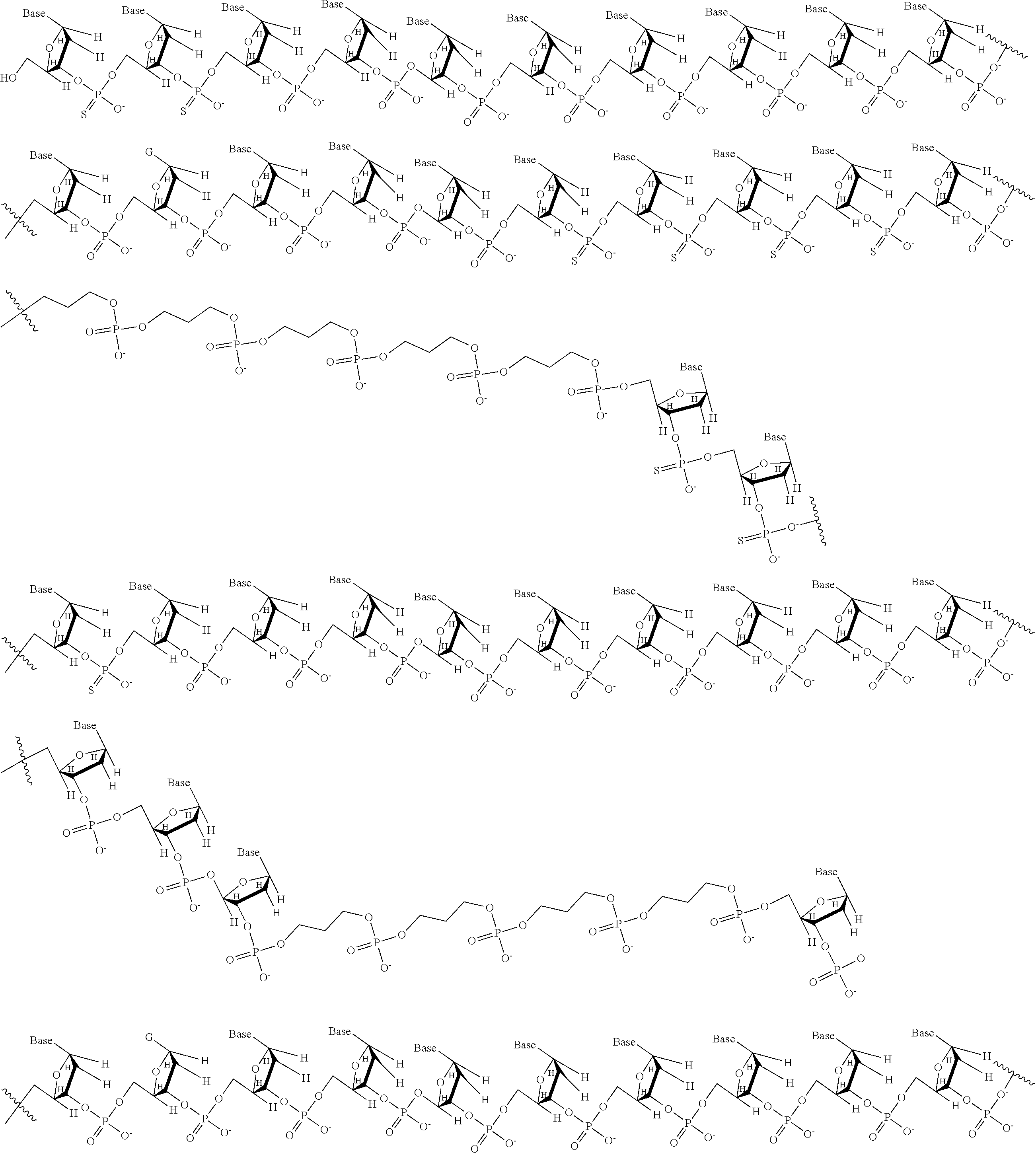

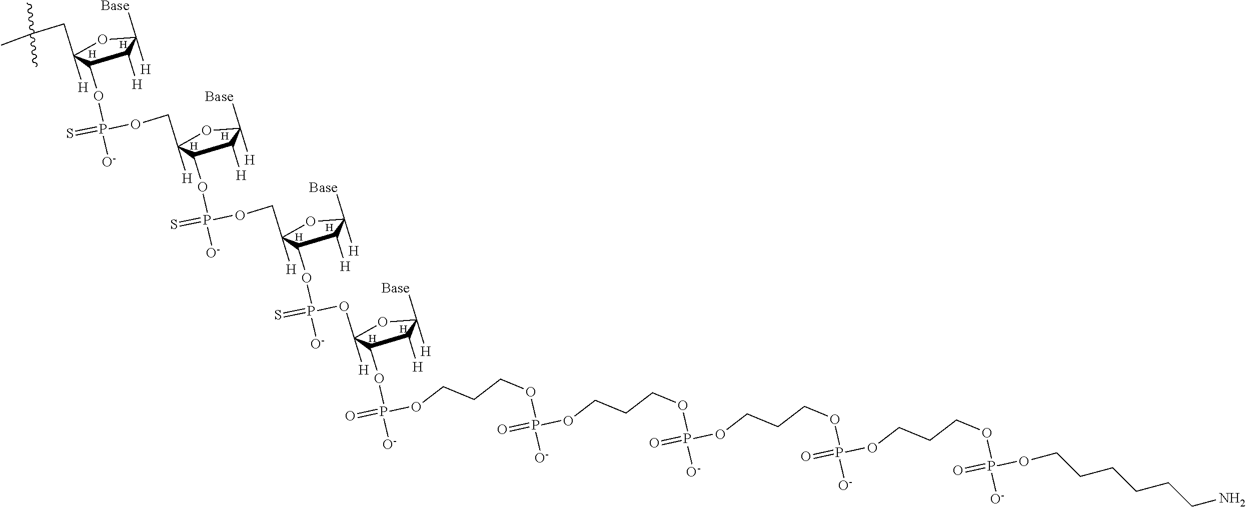

An example of a spacer connecting a terminal moiety and one nucleotide of a nucleic acid sequence, is shown below.

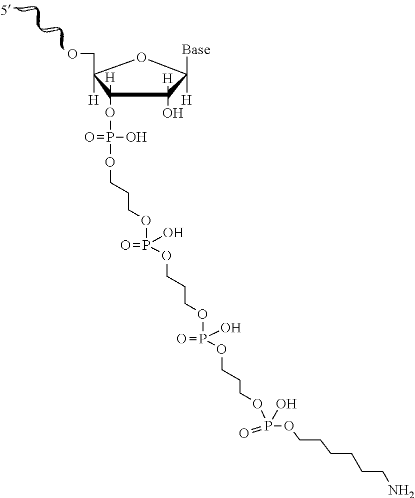

In embodiments, the spacer (e.g., first spacer, second spacer, covalent spacer, third spacer, or fourth spacer) is independently a substituted or unsubstituted C₁-C₄₀ alkylene, substituted or unsubstituted 2 to 40 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, the spacer (e.g., first spacer, second spacer, covalent spacer, third spacer, or fourth spacer) is independently a substituted or unsubstituted C₁-C₄₀ alkylene. In embodiments, the spacer (e.g., first spacer, second spacer, covalent spacer, third spacer, or fourth spacer) is independently substituted or unsubstituted 2 to 40 membered heteroalkylene. In embodiments, the spacer (e.g., first spacer, second spacer, covalent spacer, third spacer, or fourth spacer) is independently substituted or unsubstituted C₃-C₈ cycloalkylene. In embodiments, the spacer (e.g., first spacer, second spacer, covalent spacer, third spacer, or fourth spacer) is independently substituted or unsubstituted 3 to 8 membered heterocycloalkylene. In embodiments, the spacer (e.g., first spacer, second spacer, covalent spacer, third spacer, or fourth spacer) is independently substituted or unsubstituted C₆-C₁₀ arylene. In embodiments, the spacer (e.g., first spacer, second spacer, covalent spacer, third spacer, or fourth spacer) is independently substituted or unsubstituted 5 to 10 membered heteroarylene.

In embodiments, the spacer (e.g., first spacer, second spacer, covalent spacer, third spacer, or fourth spacer) is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, the spacer (e.g., first spacer, second spacer, covalent spacer, third spacer, or fourth spacer) is independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, where the spacer is substituted, it is substituted with a substituent group. In embodiments, where the spacer is substituted, it is substituted with a size-limited substituent group. In embodiments, where the spacer is substituted, it is substituted with a lower substituent group.

In embodiments, the spacer (e.g., first spacer, second spacer, covalent spacer, third spacer, or fourth spacer) is independently unsubstituted alkyl, unsubstituted heteroalkyl, unsubstituted cycloalkyl, unsubstituted heterocycloalkyl, unsubstituted aryl, or unsubstituted heteroaryl.

In embodiments, the spacer includes a first single nucleic acid strand connected to the first nucleic acid sequence capable of binding to Nuclear Factor Kappa-Light-Chain-Enhancer of Activated B Cells (NF-κB) and a second nucleic acid sequence capable of binding to Nuclear Factor Kappa-Light-Chain-Enhancer of Activated B Cells (NF-κB), wherein the first nucleic acid strand includes a nucleic acid sequence that is complementary to a nucleic acid sequence included in the second single nucleic acid strand (both single nucleic acid strands including their respective complementary sequences being collectively a “hybridized nucleic acid overhang”). In embodiments, the spacer includes a first single nucleic acid strand connected to the first NF-κB-binding nucleic acid sequence and a second single nucleic acid strand connected to the NF-κB-binding nucleic acid sequence, wherein the first nucleic acid strand includes a nucleic acid sequence that is complementary to a nucleic acid sequence included in the second single nucleic acid strand (both single nucleic acid strands including their respective complementary sequences being collectively a “hybridized nucleic acid overhang”). In embodiments, the hybridized nucleic acid overhang is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 base pairs long. In embodiments, the complementary nucleic acid sequence in the hybridized nucleic acid overhang is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 base pairs long. In embodiments, the first and second single nucleic acid strands in the hybridized nucleic acid overhang are complementary throughout their entire lengths. In embodiments, the spacer (e.g., first spacer, second spacer, covalent spacer, third spacer, or fourth spacer) is independently a substituted C₁-C₂₀ alkylene. In embodiments, the spacer (e.g., first spacer, second spacer, covalent spacer, third spacer, or fourth spacer) is independently a substituted linear C₁-C₂₀ alkylene. In embodiments, the spacer (e.g., first spacer, second spacer, covalent spacer, third spacer, or fourth spacer) is independently a substituted C₃-C₂₁ alkylene. In embodiments, the spacer (e.g., first spacer, second spacer, covalent spacer, third spacer, or fourth spacer) is independently a substituted C₃-C₁₈ alkylene. In embodiments, the spacer (e.g., first spacer, second spacer, covalent spacer, third spacer, or fourth spacer) is independently a substituted linear C₃-C₁₅ alkylene. In embodiments, the spacer (e.g., first spacer, second spacer, covalent spacer, third spacer, or fourth spacer) is independently a substituted linear C₆-C₂₁ alkylene. In embodiments, the spacer (e.g., first spacer, second spacer, covalent spacer, third spacer, or fourth spacer) is independently a substituted linear C₉-C₂₁ alkylene. In embodiments, the spacer (e.g., first spacer, second spacer, covalent spacer, third spacer, or fourth spacer) is independently a substituted linear C₉-C₁₈ alkylene. In embodiments, the spacer (e.g., first spacer, second spacer, covalent spacer, third spacer, or fourth spacer) is independently a substituted linear C₉-C₁₅ alkylene. In embodiments, the spacer (e.g., first spacer, second spacer, covalent spacer, third spacer, or fourth spacer) is independently a substituted linear C₁₂-C₁₅ alkylene. In embodiments, the spacer (e.g., first spacer, second spacer, covalent spacer, third spacer, or fourth spacer) is independently a substituted linear C₁₂ alkylene. In embodiments, the spacer (e.g., first spacer, second spacer, covalent spacer, third spacer, or fourth spacer) is independently a substituted linear C₁₃ alkylene. In embodiments, the spacer (e.g., first spacer, second spacer, covalent spacer, third spacer, or fourth spacer) is independently a substituted linear C₁₄ alkylene. In embodiments, the spacer (e.g., first spacer, second spacer, covalent spacer, third spacer, or fourth spacer) is independently a substituted linear C₁₅ alkylene. In embodiments, the spacer (e.g., first spacer, second spacer, covalent spacer, third spacer, or fourth spacer) is independently an unsubstituted C₁-C₂₀ alkylene. In embodiments, the spacer (e.g., first spacer, second spacer, covalent spacer, third spacer, or fourth spacer) is independently an unsubstituted linear C₁-C₂₀ alkylene. In embodiments, the spacer (e.g., first spacer, second spacer, covalent spacer, third spacer, or fourth spacer) is independently an unsubstituted C₃-C₂₁ alkylene. In embodiments, the spacer (e.g., first spacer, second spacer, covalent spacer, third spacer, or fourth spacer) is independently an unsubstituted C₃-C₁₈ alkylene. In embodiments, the spacer (e.g., first spacer, second spacer, covalent spacer, third spacer, or fourth spacer) is independently an unsubstituted linear C₃-C₁₅ alkylene. In embodiments, the spacer (e.g., first spacer, second spacer, covalent spacer, third spacer, or fourth spacer) is independently an unsubstituted linear C₆-C₂₁ alkylene. In embodiments, the spacer (e.g., first spacer, second spacer, covalent spacer, third spacer, or fourth spacer) is independently an unsubstituted linear C₉-C₂₁ alkylene. In embodiments, the spacer (e.g., first spacer, second spacer, covalent spacer, third spacer, or fourth spacer) is independently an unsubstituted linear C₉-C₁₈ alkylene. In embodiments, the spacer (e.g., first spacer, second spacer, covalent spacer, third spacer, or fourth spacer) is independently an unsubstituted linear C₉-C₁₅ alkylene. In embodiments, the spacer (e.g., first spacer, second spacer, covalent spacer, third spacer, or fourth spacer) is independently an unsubstituted linear C₁₂-C₁₅ alkylene. In embodiments, the spacer (e.g., first spacer, second spacer, covalent spacer, third spacer, or fourth spacer) is independently an unsubstituted linear C₁₂ alkylene. In embodiments, the spacer (e.g., first spacer, second spacer, covalent spacer, third spacer, or fourth spacer) is independently an unsubstituted linear C₁₃ alkylene. In embodiments, the spacer (e.g., first spacer, second spacer, covalent spacer, third spacer, or fourth spacer) is independently an unsubstituted linear C₁₄ alkylene. In embodiments, the spacer (e.g., first spacer, second spacer, covalent spacer, third spacer, or fourth spacer) is independently an unsubstituted linear C₁₅ alkylene. A NF-κB-binding nucleic acid (e.g. DNA) sequence is a nucleic acid (e.g. DNA) including phosphodiester linkages, phosphodiester derivative linkages, and/or nucleic acid analogs, capable of binding NF-κB. In embodiments, the spacer (e.g., first spacer, second spacer, covalent spacer, third spacer, or fourth spacer) is independently a substituted 2 to 40 membered heteroalkylene. In embodiments, the spacer (e.g., first spacer, second spacer, covalent spacer, third spacer, or fourth spacer) is independently a substituted 10 to 50 membered heteroalkylene. In embodiments, the spacer (e.g., first spacer, second spacer, covalent spacer, third spacer, or fourth spacer) is independently a substituted 20 to 40 membered heteroalkylene. In embodiments, the spacer (e.g., first spacer, second spacer, covalent spacer, third spacer, or fourth spacer) is independently a substituted 25 to 40 membered heteroalkylene. In embodiments, the spacer (e.g., first spacer, second spacer, covalent spacer, third spacer, or fourth spacer) is independently a substituted 30 to 40 membered heteroalkylene. In embodiments, the spacer (e.g., first spacer, second spacer, covalent spacer, third spacer, or fourth spacer) is independently a substituted linear 2 to 40 membered heteroalkylene. In embodiments, the spacer (e.g., first spacer, second spacer, covalent spacer, third spacer, or fourth spacer) is a substituted linear 10 to 50 membered heteroalkylene. In embodiments, the spacer (e.g., first spacer, second spacer, covalent spacer, third spacer, or fourth spacer) is independently a substituted linear 20 to 40 membered heteroalkylene. In embodiments, the spacer (e.g., first spacer, second spacer, covalent spacer, third spacer, or fourth spacer) is independently a substituted linear 25 to 40 membered heteroalkylene. In embodiments, the spacer (e.g., first spacer, second spacer, covalent spacer, third spacer, or fourth spacer) is independently a substituted linear 30 to 40 membered heteroalkylene. In embodiments, the spacer (e.g., first spacer, second spacer, covalent spacer, third spacer, or fourth spacer) is independently an unsubstituted 2 to 40 membered heteroalkylene. In embodiments, the spacer (e.g., first spacer, second spacer, covalent spacer, third spacer, or fourth spacer) is independently an unsubstituted 10 to 50 membered heteroalkylene. In embodiments, the spacer (e.g., first spacer, second spacer, covalent spacer, third spacer, or fourth spacer) is independently an unsubstituted 20 to 40 membered heteroalkylene. In embodiments, the spacer (e.g., first spacer, second spacer, covalent spacer, third spacer, or fourth spacer) is independently an unsubstituted 25 to 40 membered heteroalkylene. In embodiments, the spacer (e.g., first spacer, second spacer, covalent spacer, third spacer, or fourth spacer) is independently an unsubstituted 30 to 40 membered heteroalkylene. In embodiments, the spacer (e.g., first spacer, second spacer, covalent spacer, third spacer, or fourth spacer) is independently an unsubstituted linear 2 to 40 membered heteroalkylene. In embodiments, the spacer (e.g., first spacer, second spacer, covalent spacer, third spacer, or fourth spacer) is an unsubstituted linear 10 to 50 membered heteroalkylene. In embodiments, the spacer (e.g., first spacer, second spacer, covalent spacer, third spacer, or fourth spacer) is independently an unsubstituted linear 20 to 40 membered heteroalkylene. In embodiments, the spacer (e.g., first spacer, second spacer, covalent spacer, third spacer, or fourth spacer) is independently an unsubstituted linear 25 to 40 membered heteroalkylene. In embodiments, the spacer (e.g., first spacer, second spacer, covalent spacer, third spacer, or fourth spacer) is independently an unsubstituted linear 30 to 40 membered heteroalkylene.

In embodiments, the spacer is a “third nucleic acid sequence”. In embodiments, the “third nucleic acid sequence may be 1 to 100 nucleotides in length. In embodiments, the “third nucleic acid sequence may be 1 to 90 nucleotides in length. In embodiments, the “third nucleic acid sequence may be 1 to 80 nucleotides in length. In embodiments, the “third nucleic acid sequence may be 1 to 70 nucleotides in length. In embodiments, the “third nucleic acid sequence may be 1 to 60 nucleotides in length. In embodiments, the “third nucleic acid sequence may be 1 to 50 nucleotides in length. In embodiments, the “third nucleic acid sequence may be 1 to 40 nucleotides in length. In embodiments, the “third nucleic acid sequence may be 1 to 30 nucleotides in length. In embodiments, the “third nucleic acid sequence may be 1 to 20 nucleotides in length. In embodiments, the “third nucleic acid sequence may be 1 to 10 nucleotides in length. In embodiments, the “third nucleic acid sequence may be 1 to 5 nucleotides in length. In embodiments, the “third nucleic acid sequence may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In embodiments, the compound further includes a phosphorothioate linkage in the first nucleic acid sequence or the second nucleic acid sequence. In embodiments, the compound further includes a phosphorothioate linkage in the first nucleic acid sequence. In embodiments, the compound further includes a phosphorothioate linkage in the second nucleic acid sequence. In embodiments, the compound further includes a plurality of phosphorothioate linkages.

In embodiments, the Toll-like receptor protein is human Toll-like receptor 3, Toll-like receptor 7, Toll-like receptor 8, or Toll-like receptor 9. In embodiments, the Toll-like receptor protein is human Toll-like receptor 3. In embodiments, the Toll-like receptor protein is human Toll-like receptor 7. In embodiments, the Toll-like receptor protein is human Toll-like receptor 8. In embodiments, the Toll-like receptor protein is human Toll-like receptor 9 (TLR9).

In embodiments, the second nucleic acid sequence includes an aliphatic spacer, CpG motif, a GpC motif, or a phosphorothioated nucleic acid sequence including at least 10 nucleotides; or corresponding in length to 10 nucleotides. In embodiments, the second nucleic acid sequence includes an aliphatic spacer, CpG motif, a GpC motif, or a phosphorothioated nucleic acid sequence including at least 10 nucleotides. In embodiments, the second nucleic acid sequence includes an aliphatic spacer, CpG motif, a GpC motif, or a phosphorothioated nucleic acid sequence corresponding in length to 10 nucleotides. In embodiments, the second nucleic acid sequence includes a CpG motif, a GpC motif, or a phosphorothioated nucleic acid sequence including at least 10 nucleotides. In embodiments, the second nucleic acid sequence includes a CpG motif. In embodiments, the second nucleic acid sequence includes a GpC motif. In embodiments, the second nucleic acid sequence includes a phosphorothioated nucleic acid sequence including at least 10 nucleotides. In embodiments, the second nucleic acid sequence includes an unmethylated CpG motif.

An aliphatic spacer is a compound having the formula of a hydrocarbon between two phosphodiesters (or phosphodiester derivatives), two phosphates (or phosphate derivatives), or a mixture thereof, where the aliphatic spacer can be independently monovalent or divalent.

In embodiments, the second nucleic acid sequence includes a Class A CpG DNA sequence, Class B CpG DNA sequence, or Class C CpG DNA sequence. In embodiments, the second nucleic acid sequence includes a Class A CpG DNA sequence. In embodiments, the second nucleic acid sequence includes a Class B CpG DNA sequence. In embodiments, the second nucleic acid sequence includes a Class C CpG DNA sequence.

In embodiments, the compound has the sequence: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, or SEQ ID NO: 21. In embodiments, the compound has the sequence: SEQ ID NO: 1. In embodiments, the compound has the sequence SEQ ID NO: 2. In embodiments, the compound has the sequence SEQ ID NO: 3. In embodiments, the compound has the sequence SEQ ID NO: 4. In embodiments, the compound has the sequence SEQ ID NO: 5. In embodiments, the compound has the sequence SEQ ID NO: 6. In embodiments, the compound has the sequence SEQ ID NO: 7. In embodiments, the compound has the sequence SEQ ID NO: 8. In embodiments, the compound has the sequence SEQ ID NO: 9. In embodiments, the compound has the sequence SEQ ID NO: 10. In embodiments, the compound has the sequence SEQ ID NO: 11. In embodiments, the compound has the sequence SEQ ID NO: 12. In embodiments, the compound has the sequence SEQ ID NO: 13. In embodiments, the compound has the sequence SEQ ID NO: 14. In embodiments, the compound has the sequence SEQ ID NO: 15. In embodiments, the compound has the sequence SEQ ID NO: 16. In embodiments, the compound has the sequence SEQ ID NO: 17. In embodiments, the compound has the sequence SEQ ID NO: 21.

In an aspect is provided a nucleic acid sequence (e.g., first nucleic acid sequence, second nucleic acid sequence, third nucleic acid sequence, first NF-κB binding site nucleic acid sequence, second NF-κB binding site nucleic acid sequence, first TLR binding site nucleic acid sequence, second TLR site nucleic acid sequence, spacer (e.g., first spacer, second spacer, covalent spacer, third spacer, or fourth spacer), or terminal moiety) having at least 80% identity to a sequence (e.g. a first nucleic acid sequence, second nucleic acid sequence, third nucleic acid sequence, first NF-κB binding site nucleic acid sequence, second NF-κB binding site nucleic acid sequence, first TLR binding site nucleic acid sequence, second TLR site nucleic acid sequence, spacer (e.g., first spacer, second spacer, covalent spacer, third spacer, or fourth spacer), or terminal moiety respectively) disclosed herein (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17, or SEQ ID NO: 21); or portion thereof (e.g., first nucleic acid sequence, second nucleic acid sequence, third nucleic acid sequence, first NF-κB binding site nucleic acid sequence, second NF-κB binding site nucleic acid sequence, first TLR binding site nucleic acid sequence, second TLR site nucleic acid sequence, spacer (e.g., first spacer, second spacer, covalent spacer, third spacer, or fourth spacer), or terminal moiety). In one example, a nucleic acid sequence as disclosed herein (e.g., first nucleic acid sequence, second nucleic acid sequence, third nucleic acid sequence, first NF-κB binding site nucleic acid sequence, second NF-κB binding site nucleic acid sequence, first TLR binding site nucleic acid sequence, second TLR site nucleic acid sequence, spacer (e.g., first spacer, second spacer, covalent spacer, third spacer, or fourth spacer), or terminal moiety) comprises sequence at least about 85% or 90% or 95% or 97% or 98% or 99% identical to a sequence disclosed herein (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, or SEQ ID NO: 21); or portion thereof (e.g., first nucleic acid sequence, second nucleic acid sequence, third nucleic acid sequence, first NF-κB binding site nucleic acid sequence, second NF-κB binding site nucleic acid sequence, first TLR binding site nucleic acid sequence, second TLR site nucleic acid sequence, spacer (e.g., first spacer, second spacer, covalent spacer, third spacer, or fourth spacer), or terminal moiety). In embodiments, a first nucleic acid sequence, second nucleic acid sequence, third nucleic acid sequence, first NF-κB binding site nucleic acid sequence, second NF-κB binding site nucleic acid sequence, first TLR binding site nucleic acid sequence, second TLR site nucleic acid sequence, spacer (e.g., first spacer, second spacer, covalent spacer, third spacer, or fourth spacer), or terminal moiety) comprises sequence at least about 85% or 90% or 95% or 97% or 98% or 99% identical to a first nucleic acid sequence, second nucleic acid sequence, third nucleic acid sequence, first NF-κB binding site nucleic acid sequence, second NF-κB binding site nucleic acid sequence, first TLR binding site nucleic acid sequence, second TLR site nucleic acid sequence, spacer (e.g., first spacer, second spacer, covalent spacer, third spacer, or fourth spacer), or terminal moiety) respectively, which is disclosed herein (e.g., included in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, or SEQ ID NO: 21). In one example, a nucleic acid sequence as disclosed herein (e.g., first nucleic acid sequence, second nucleic acid sequence, third nucleic acid sequence, first NF-κB binding site nucleic acid sequence, second NF-κB binding site nucleic acid sequence, first TLR binding site nucleic acid sequence, second TLR site nucleic acid sequence, spacer (e.g., first spacer, second spacer, covalent spacer, third spacer, or fourth spacer), or terminal moiety) is a sequence at least about 85% or 90% or 95% or 97% or 98% or 99% identical to a sequence disclosed herein (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17, or SEQ ID NO: 21; or portion thereof (e.g., first nucleic acid sequence, second nucleic acid sequence, third nucleic acid sequence, first NF-κB binding site nucleic acid sequence, second NF-κB binding site nucleic acid sequence, first TLR binding site nucleic acid sequence, second TLR site nucleic acid sequence, spacer (e.g., first spacer, second spacer, covalent spacer, third spacer, or fourth spacer), or terminal moiety). In embodiments, a first nucleic acid sequence, second nucleic acid sequence, third nucleic acid sequence, first NF-κB binding site nucleic acid sequence, second NF-κB binding site nucleic acid sequence, first TLR binding site nucleic acid sequence, second TLR site nucleic acid sequence, spacer (e.g., first spacer, second spacer, covalent spacer, third spacer, or fourth spacer), or terminal moiety) is a sequence at least about 85% or 90% or 95% or 97% or 98% or 99% identical to a first nucleic acid sequence, second nucleic acid sequence, third nucleic acid sequence, first NF-κB binding site nucleic acid sequence, second NF-κB binding site nucleic acid sequence, first TLR binding site nucleic acid sequence, second TLR site nucleic acid sequence, spacer (e.g., first spacer, second spacer, covalent spacer, third spacer, or fourth spacer), or terminal moiety) respectively, which is disclosed herein (e.g., included in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, or SEQ ID NO: 21). In embodiments, when a nucleic acid sequence as disclosed herein (e.g., first nucleic acid sequence, second nucleic acid sequence, third nucleic acid sequence, first NF-κB binding site nucleic acid sequence, second NF-κB binding site nucleic acid sequence, first TLR binding site nucleic acid sequence, second TLR site nucleic acid sequence, spacer (e.g., first spacer, second spacer, covalent spacer, third spacer, or fourth spacer), or terminal moiety) is a sequence or comprises a sequence at least about 85% or 90% or 95% or 97% or 98% or 99% identical to a sequence disclosed herein (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17, or SEQ ID NO: 21; or portion thereof (e.g., first nucleic acid sequence, second nucleic acid sequence, third nucleic acid sequence, first NF-κB binding site nucleic acid sequence, second NF-κB binding site nucleic acid sequence, first TLR binding site nucleic acid sequence, second TLR site nucleic acid sequence, spacer (e.g., first spacer, second spacer, covalent spacer, third spacer, or fourth spacer), or terminal moiety), the nucleic acid sequence % identify refers to the % identity of the nucleosides in the sequence. In embodiments the nucleic acid sequence % identity to a sequence disclosed herein includes all possible phosphodiester and/or phosphodiester derivatives bonded to each nucleoside in the nucleic acid sequence. In embodiments the nucleic acid sequence % identity to a sequence disclosed herein is measured by % identify of nucleotides (e.g., including the phosphodiester and/or phosphodiester derivatives of the sequence disclosed herein).

In embodiments, the first terminal moiety is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the first terminal moiety is a substituted or unsubstituted C₁-C₄₀ alkyl, substituted or unsubstituted 2 to 40 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, the first terminal moiety is a substituted C₁-C₄₀ alkyl, substituted 2 to 40 membered heteroalkyl, substituted C₃-C₈ cycloalkyl, substituted 3 to 8 membered heterocycloalkyl, substituted C₆-C₁₀ aryl, or substituted 5 to 10 membered heteroaryl.

In embodiments, the first terminal moiety is independently a hydrogen, monophosphate, polyphosphate, —OH, —NH₂, or

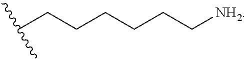 [SMILES: *CCCCCCN]

In embodiments, the first terminal moiety is independently a hydrogen. In embodiments, the first terminal moiety is independently monophosphate. In embodiments, the first terminal moiety is independently polyphosphate. In embodiments, the first terminal moiety is independently —OH. In embodiments, the terminal moiety is independently —NH₂. In embodiments, the first terminal moiety is independently

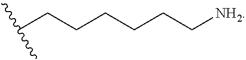 [SMILES: *CCCCCCN]

In embodiments, the first terminal moiety is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the first terminal moiety is a substituted or unsubstituted C₁-C₄₀ alkyl, substituted or unsubstituted 2 to 40 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, the first terminal moiety is a substituted C₁-C₄₀ alkyl, substituted 2 to 40 membered heteroalkyl, substituted C₃-C₈ cycloalkyl, substituted 3 to 8 membered heterocycloalkyl, substituted C₆-C₁₀ aryl, or substituted 5 to 10 membered heteroaryl. In embodiments, the first terminal moiety is an R¹-substituted C₁-C₄₀ alkyl, R¹-substituted 2 to 40 membered heteroalkyl, R¹-substituted C₃-C₈ cycloalkyl, R¹-substituted 3 to 8 membered heterocycloalkyl, R¹-substituted C₆-C₁₀ aryl, or R¹-substituted 5 to 10 membered heteroaryl. In embodiments, the first terminal moiety is an R¹-substituted C₁-C₄₀ alkyl. In embodiments, the first terminal moiety is an -(unsubstituted C₁-C₄₀ alkylene)-R¹. In embodiments, the first terminal moiety is an -(unsubstituted linear C₁-C₄₀ alkylene)-R¹. In embodiments, the first terminal moiety is an -(unsubstituted C₃-C₂₁ alkylene)-R¹. In embodiments, the first terminal moiety is an -(unsubstituted C₃-C₁₅ alkylene)-R¹. In embodiments, the first terminal moiety is an -(unsubstituted linear C₃-C₁₅ alkylene)-R¹. In embodiments, the first terminal moiety is an -(unsubstituted linear C₆-C₂₁ alkylene)-R¹. In embodiments, the first terminal moiety is an -(unsubstituted linear C₉-C₂₁ alkylene)-R¹. In embodiments, the first terminal moiety is an -(unsubstituted linear C₉-C₁₈ alkylene)-R¹. In embodiments, the first terminal moiety is an -(unsubstituted linear C₉-C₁₅ alkylene)-R¹. In embodiments, the first terminal moiety is an -(unsubstituted linear C₁₂-C₁₅ alkylene)-R¹. In embodiments, the first terminal moiety is an -(unsubstituted linear C₁₂ alkylene)-R¹. In embodiments, the first terminal moiety is an -(unsubstituted linear C₁₃ alkylene)-R¹. In embodiments, the first terminal moiety is an -(unsubstituted linear C₁₄ alkylene)-R¹. In embodiments, the first terminal moiety is an -(unsubstituted linear Cis alkylene)-R¹. In embodiments, the first terminal moiety is an R¹-substituted 2 to 40 membered heteroalkyl. In embodiments, the first terminal moiety is an -(unsubstituted 2 to 40 membered heteroalkylene)-R¹. In embodiments, the first terminal moiety is a -(substituted linear 2 to 40 membered heteroalkylene)-R¹. In embodiments, the first terminal moiety is a -(substituted 5 to 40 membered heteroalkylene)-R¹. In embodiments, the first terminal moiety is a -(substituted 10 to 40 membered heteroalkylene)-R¹. In embodiments, the first terminal moiety is a -(substituted 15 to 40 membered heteroalkylene)-R¹. In embodiments, the first terminal moiety is a -(substituted 20 to 40 membered heteroalkylene)-R¹. In embodiments, the first terminal moiety is a -(substituted 30 to 40 membered heteroalkylene)-R¹. In embodiments, the first terminal moiety is a -(substituted 2 to 35 membered heteroalkylene)-R¹. In embodiments, the first terminal moiety is a -(substituted 2 to 30 membered heteroalkylene)-R¹. In embodiments, the first terminal moiety is a -(substituted 2 to 25 membered heteroalkylene)-R¹. In embodiments, the first terminal moiety is a -(substituted 2 to 20 membered heteroalkylene)-R¹. In embodiments, the first terminal moiety is a -(substituted 2 to 10 membered heteroalkylene)-R¹. In embodiments, the first terminal moiety is a -(substituted 2 to 50 membered heteroalkylene)-R¹. In embodiments, the first terminal moiety is a -(substituted 2 to 60 membered heteroalkylene)-R¹. In embodiments, the first terminal moiety is an R¹-substituted 2 to 40 membered heteroalkyl. In embodiments, the first terminal moiety is an R¹-substituted 10 to 50 membered heteroalkyl. In embodiments, the first terminal moiety is an R¹-substituted 20 to 40 membered heteroalkyl. In embodiments, the first terminal moiety is an R¹-substituted 25 to 40 membered heteroalkyl. In embodiments, the first terminal moiety is an R¹-substituted 30 to 40 membered heteroalkyl. In embodiments, the first terminal moiety is a substituted 2 to 40 membered heteroalkyl. In embodiments, the first terminal moiety is a substituted 10 to 50 membered heteroalkyl. In embodiments, the first terminal moiety is a substituted 20 to 40 membered heteroalkyl. In embodiments, the first terminal moiety is a substituted 25 to 40 membered heteroalkyl. In embodiments, the first terminal moiety is a substituted 30 to 40 membered heteroalkyl.

R¹ is halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered), detectable moiety, or a therapeutic moiety. In embodiments, R¹ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered), detectable moiety, or a therapeutic moiety. In embodiments, R¹ is a detectable moiety. In embodiments, the detectable moiety is a fluorescent dye, electron-dense reagent, enzyme, biotin, digoxigenin, paramagnetic molecule, paramagnetic nanoparticle, contrast agent, magnetic resonance contrast agent, X-ray contrast agent, Gadolinium, radioisotope, radionuclide, fluorodeoxyglucose, gamma ray emitting radionuclide, positron-emitting radionuclide, biocolloid, microbubble, iodinated contrast agent, barium sulfate, thorium dioxide, gold, gold nanoparticle, gold nanoparticle aggregate, fluorophore, two-photon fluorophore, hapten, protein, or fluorescent moiety. In embodiments, R¹ is a therapeutic moiety (e.g. anti-cancer agent or anti-viral agent).

In embodiments, the first terminal moiety is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, the first terminal moiety is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, where the first terminal moiety is substituted, it is substituted with a substituent group. In embodiments, where the first terminal moiety is substituted, it is substituted with a size-limited substituent group. In embodiments, where the first terminal moiety is substituted, it is substituted with a lower substituent group.

In embodiments, the first terminal moiety is unsubstituted alkyl, unsubstituted heteroalkyl, unsubstituted cycloalkyl, unsubstituted heterocycloalkyl, unsubstituted aryl, or unsubstituted heteroaryl.

In embodiments, the second terminal moiety is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the second terminal moiety is a substituted or unsubstituted C₁-C₄₀ alkyl, substituted or unsubstituted 2 to 40 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, the second terminal moiety is a substituted C₁-C₄₀ alkyl, substituted 2 to 40 membered heteroalkyl, substituted C₃-C₈ cycloalkyl, substituted 3 to 8 membered heterocycloalkyl, substituted C₆-C₁₀ aryl, or substituted 5 to 10 membered heteroaryl.

In embodiments, the second terminal moiety is independently a hydrogen, monophosphate, polyphosphate, —OH, —NH₂, or

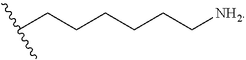 [SMILES: *CCCCCCN]

In embodiments, the second terminal moiety is independently a hydrogen. In embodiments, the second terminal moiety is independently monophosphate. In embodiments, the second terminal moiety is independently polyphosphate. In embodiments, the second terminal moiety is independently —OH. In embodiments, the terminal moiety is independently —NH₂. In embodiments, the second terminal moiety is independently

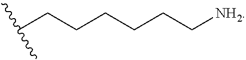 [SMILES: *CCCCCCN]

In embodiments, the second terminal moiety is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the second terminal moiety is a substituted or unsubstituted C₁-C₄₀ alkyl, substituted or unsubstituted 2 to 40 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, the second terminal moiety is a substituted C₁-C₄₀ alkyl, substituted 2 to 40 membered heteroalkyl, substituted C₃-C₈ cycloalkyl, substituted 3 to 8 membered heterocycloalkyl, substituted C₆-C₁₀ aryl, or substituted 5 to 10 membered heteroaryl. In embodiments, the second terminal moiety is an R¹-substituted C₁-C₄₀ alkyl, R¹-substituted 2 to 40 membered heteroalkyl, R¹-substituted C₃-C₈ cycloalkyl, R¹-substituted 3 to 8 membered heterocycloalkyl, R¹-substituted C₆-C₁₀ aryl, or R¹-substituted 5 to 10 membered heteroaryl. In embodiments, the second terminal moiety is an R¹-substituted C₁-C₄₀ alkyl. In embodiments, the second terminal moiety is an -(unsubstituted C₁-C₄₀ alkylene)-R¹. In embodiments, the second terminal moiety is an -(unsubstituted linear C₁-C₄₀ alkylene)-R¹. In embodiments, the second terminal moiety is an -(unsubstituted C₃-C₂₁ alkylene)-R¹. In embodiments, the second terminal moiety is an -(unsubstituted C₃-C₁₅ alkylene)-R¹. In embodiments, the second terminal moiety is an -(unsubstituted linear C₃-C₁₅ alkylene)-R¹. In embodiments, the second terminal moiety is an -(unsubstituted linear C₆-C₂₁ alkylene)-R¹. In embodiments, the second terminal moiety is an -(unsubstituted linear C₉-C₂₁ alkylene)-R¹. In embodiments, the second terminal moiety is an -(unsubstituted linear C₉-C₁₈ alkylene)-R¹. In embodiments, the second terminal moiety is an -(unsubstituted linear C₉-C₁₅ alkylene)-R¹. In embodiments, the second terminal moiety is an -(unsubstituted linear C₁₂-C₁₅ alkylene)-R¹. In embodiments, the second terminal moiety is an -(unsubstituted linear C₁₂ alkylene)-R¹. In embodiments, the second terminal moiety is an -(unsubstituted linear C₁₃ alkylene)-R¹. In embodiments, the second terminal moiety is an -(unsubstituted linear C₁₄ alkylene)-R¹. In embodiments, the second terminal moiety is an -(unsubstituted linear C₁₅ alkylene)-R¹. In embodiments, the second terminal moiety is an R¹-substituted 2 to 40 membered heteroalkyl. In embodiments, the second terminal moiety is an -(unsubstituted 2 to 40 membered heteroalkylene)-R¹. In embodiments, the second terminal moiety is a -(substituted linear 2 to 40 membered heteroalkylene)-R¹. In embodiments, the second terminal moiety is a -(substituted 5 to 40 membered heteroalkylene)-R¹. In embodiments, the second terminal moiety is a -(substituted 10 to 40 membered heteroalkylene)-R¹. In embodiments, the second terminal moiety is a -(substituted 15 to 40 membered heteroalkylene)-R¹. In embodiments, the second terminal moiety is a -(substituted 20 to 40 membered heteroalkylene)-R¹. In embodiments, the second terminal moiety is a -(substituted 30 to 40 membered heteroalkylene)-R¹. In embodiments, the second terminal moiety is a -(substituted 2 to 35 membered heteroalkylene)-R¹. In embodiments, the second terminal moiety is a -(substituted 2 to 30 membered heteroalkylene)-R¹. In embodiments, the second terminal moiety is a -(substituted 2 to 25 membered heteroalkylene)-R¹. In embodiments, the second terminal moiety is a -(substituted 2 to 20 membered heteroalkylene)-R¹. In embodiments, the second terminal moiety is a -(substituted 2 to 10 membered heteroalkylene)-R¹. In embodiments, the second terminal moiety is a -(substituted 2 to 50 membered heteroalkylene)-R¹. In embodiments, the second terminal moiety is a -(substituted 2 to 60 membered heteroalkylene)-R¹. In embodiments, the second terminal moiety is an R¹-substituted 2 to 40 membered heteroalkyl. In embodiments, the second terminal moiety is an R¹-substituted 10 to 50 membered heteroalkyl. In embodiments, the second terminal moiety is an R¹-substituted 20 to 40 membered heteroalkyl. In embodiments, the second terminal moiety is an R¹-substituted 25 to 40 membered heteroalkyl. In embodiments, the second terminal moiety is an R¹-substituted 30 to 40 membered heteroalkyl. In embodiments, the second terminal moiety is a substituted 2 to 40 membered heteroalkyl. In embodiments, the second terminal moiety is a substituted 10 to 50 membered heteroalkyl. In embodiments, the second terminal moiety is a substituted 20 to 40 membered heteroalkyl. In embodiments, the second terminal moiety is a substituted 25 to 40 membered heteroalkyl. In embodiments, the second terminal moiety is a substituted 30 to 40 membered heteroalkyl.

In embodiments, the second terminal moiety is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, the second terminal moiety is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, where the second terminal moiety is substituted, it is substituted with a substituent group. In embodiments, where the second terminal moiety is substituted, it is substituted with a size-limited substituent group. In embodiments, where the second terminal moiety is substituted, it is substituted with a lower substituent group.

In embodiments, the second terminal moiety is unsubstituted alkyl, unsubstituted heteroalkyl, unsubstituted cycloalkyl, unsubstituted heterocycloalkyl, unsubstituted aryl, or unsubstituted heteroaryl.

In embodiments, the compound includes a covalently bonded terminal moiety. In embodiments, the compound is covalently bonded to a terminal moiety.

A terminal moiety is hydrogen, a nucleic acid sequence, DNA sequence, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, a terminal moiety is a chemically reactive moiety, detectable moiety, therapeutic moiety (e.g. anti-cancer agent or anti-viral agent), nucleic acid sequence, DNA sequence, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, a terminal moiety is a detectable moiety. In embodiments, the detectable moiety is a fluorescent dye, electron-dense reagent, enzyme, biotin, digoxigenin, paramagnetic molecule, paramagnetic nanoparticle, contrast agent, magnetic resonance contrast agent, X-ray contrast agent, Gadolinium, radioisotope, radionuclide, fluorodeoxyglucose, gamma ray emitting radionuclide, positron-emitting radionuclide, biocolloid, microbubble, iodinated contrast agent, barium sulfate, thorium dioxide, gold, gold nanoparticle, gold nanoparticle aggregate, fluorophore, two-photon fluorophore, hapten, protein, or fluorescent moiety. In embodiments, a terminal moiety is a therapeutic moiety (e.g. anti-cancer agent or anti-viral agent).

In embodiments, the terminal moiety is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the terminal moiety is a substituted or unsubstituted C₁-C₄₀ alkyl, substituted or unsubstituted 2 to 40 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, the terminal moiety is a substituted C₁-C₄₀ alkyl, substituted 2 to 40 membered heteroalkyl, substituted C₃-C₈ cycloalkyl, substituted 3 to 8 membered heterocycloalkyl, substituted C₆-C₁₀ aryl, or substituted 5 to 10 membered heteroaryl.

In embodiments, the terminal moiety is independently a hydrogen, monophosphate, polyphosphate, —OH, —NH₂, or

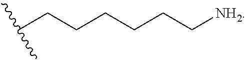 [SMILES: *CCCCCCN]

In embodiments, the terminal moiety is independently a hydrogen. In embodiments, the terminal moiety is independently monophosphate. In embodiments, the terminal moiety is independently polyphosphate. In embodiments, the terminal moiety is independently —OH. In embodiments, the terminal moiety is independently —NH₂. In embodiments, the terminal moiety is independently

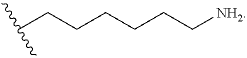 [SMILES: *CCCCCCN]

In embodiments, the terminal moiety is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the terminal moiety is a substituted or unsubstituted C₁-C₄₀ alkyl, substituted or unsubstituted 2 to 40 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, the terminal moiety is a substituted C₁-C₄₀ alkyl, substituted 2 to 40 membered heteroalkyl, substituted C₃-C₈ cycloalkyl, substituted 3 to 8 membered heterocycloalkyl, substituted C₆-C₁₀ aryl, or substituted 5 to 10 membered heteroaryl. In embodiments, the terminal moiety is an R¹-substituted C₁-C₄₀ alkyl, R¹-substituted 2 to 40 membered heteroalkyl, R¹-substituted C₃-C₈ cycloalkyl, R¹-substituted 3 to 8 membered heterocycloalkyl, R¹-substituted C₆-C₁₀ aryl, or R¹-substituted 5 to 10 membered heteroaryl. In embodiments, the terminal moiety is an R¹-substituted C₁-C₄₀ alkyl. In embodiments, the terminal moiety is an -(unsubstituted C₁-C₄₀ alkylene)-R¹. In embodiments, the terminal moiety is an -(unsubstituted linear C₁-C₄₀ alkylene)-R¹. In embodiments, the terminal moiety is an -(unsubstituted C₃-C₂₁ alkylene)-R¹. In embodiments, the terminal moiety is an -(unsubstituted C₃-C₁₈ alkylene)-R¹. In embodiments, the terminal moiety is an -(unsubstituted linear C₃-C₁₅ alkylene)-R¹. In embodiments, the terminal moiety is an -(unsubstituted linear C₆-C₂₁ alkylene)-R¹. In embodiments, the terminal moiety is an -(unsubstituted linear C₉-C₂₁ alkylene)-R¹. In embodiments, the terminal moiety is an -(unsubstituted linear C₉-C₁₈ alkylene)-R¹. In embodiments, the terminal moiety is an -(unsubstituted linear C₉-C₁₅ alkylene)-R¹. In embodiments, the terminal moiety is an -(unsubstituted linear C₁₂-C₁₅ alkylene)-R¹. In embodiments, the terminal moiety is an -(unsubstituted linear C₁₂ alkylene)-R¹. In embodiments, the terminal moiety is an -(unsubstituted linear C₁₃ alkylene)-R¹. In embodiments, the terminal moiety is an -(unsubstituted linear C₁₄ alkylene)-R¹. In embodiments, the terminal moiety is an -(unsubstituted linear C₁₅ alkylene)-R¹. In embodiments, the terminal moiety is an R¹-substituted 2 to 40 membered heteroalkyl. In embodiments, the terminal moiety is an -(unsubstituted 2 to 40 membered heteroalkylene)-R¹. In embodiments, the terminal moiety is a -(substituted linear 2 to 40 membered heteroalkylene)-R¹. In embodiments, the terminal moiety is a -(substituted 5 to 40 membered heteroalkylene)-R¹. In embodiments, the terminal moiety is a -(substituted 10 to 40 membered heteroalkylene)-R¹. In embodiments, the terminal moiety is a -(substituted 15 to 40 membered heteroalkylene)-R¹. In embodiments, the terminal moiety is a -(substituted 20 to 40 membered heteroalkylene)-R¹. In embodiments, the terminal moiety is a -(substituted 30 to 40 membered heteroalkylene)-R¹. In embodiments, the terminal moiety is a -(substituted 2 to 35 membered heteroalkylene)-R¹. In embodiments, the terminal moiety is a -(substituted 2 to 30 membered heteroalkylene)-R¹. In embodiments, the terminal moiety is a -(substituted 2 to 25 membered heteroalkylene)-R¹. In embodiments, the terminal moiety is a -(substituted 2 to 20 membered heteroalkylene)-R¹. In embodiments, the terminal moiety is a -(substituted 2 to 10 membered heteroalkylene)-R¹. In embodiments, the terminal moiety is a -(substituted 2 to 50 membered heteroalkylene)-R¹. In embodiments, the terminal moiety is a -(substituted 2 to 60 membered heteroalkylene)-R¹. In embodiments, the terminal moiety is an R¹-substituted 2 to 40 membered heteroalkyl. In embodiments, the terminal moiety is an R¹-substituted 10 to 50 membered heteroalkyl. In embodiments, the terminal moiety is an R¹-substituted 20 to 40 membered heteroalkyl. In embodiments, the terminal moiety is an R¹-substituted 25 to 40 membered heteroalkyl. In embodiments, the terminal moiety is an R¹-substituted 30 to 40 membered heteroalkyl. In embodiments, the terminal moiety is a substituted 2 to 40 membered heteroalkyl. In embodiments, the terminal moiety is a substituted 10 to 50 membered heteroalkyl. In embodiments, the terminal moiety is a substituted 20 to 40 membered heteroalkyl. In embodiments, the terminal moiety is a substituted 25 to 40 membered heteroalkyl. In embodiments, the terminal moiety is a substituted 30 to 40 membered heteroalkyl.

In embodiments, the terminal moiety is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, the terminal moiety is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, where the terminal moiety is substituted, it is substituted with a substituent group. In embodiments, where the terminal moiety is substituted, it is substituted with a size-limited substituent group. In embodiments, where terminal moiety is substituted, it is substituted with a lower substituent group.

In embodiments, the terminal moiety is unsubstituted alkyl, unsubstituted heteroalkyl, unsubstituted cycloalkyl, unsubstituted heterocycloalkyl, unsubstituted aryl, or unsubstituted heteroaryl.

In embodiments, the Toll-like receptor protein is human Toll-like receptor 3 (TLR3), Toll-like receptor 7 (TLR7), Toll-like receptor 8 (TLR8), or Toll-like receptor 9 (TLR9). In embodiments, the Toll-like receptor protein is human Toll-like receptor 3 (TLR3). In embodiments, the Toll-like receptor protein is Toll-like receptor 7 (TLR7). In embodiments, the Toll-like receptor protein is Toll-like receptor 8 (TLR8). In embodiments, the Toll-like receptor protein is Toll-like receptor 9 (TLR9).

In embodiments, the second nucleic acid sequence includes an unmethylated CpG motif.

In embodiments, the compound includes a CpG motif. In embodiments, the compound includes an unmethylated CpG motif. In embodiments, the compound includes a CpG motif wherein the CpG is not methylated. In embodiments, the compound includes a nucleic acid sequence capable of forming a G-quadruplex. In embodiments, the compound includes a DNA sequence capable of forming a G-quadruplex. In embodiments, the compound includes a Class A CpG DNA sequence. In embodiments, the compound includes a Class B CpG DNA sequence. In embodiments, the compound includes a C-type CpG DNA sequence. In embodiments, the compound binds an endosomal TLR. In embodiments, the compound preferentially binds an endosomal TLR over other TLR. In embodiments, the compound specifically binds an endosomal TLR. In embodiments, the compound binds TLR3. In embodiments, the compound preferentially binds TLR3 over other TLR. In embodiments, the compound specifically binds TLR3. In embodiments, the compound binds TLR7. In embodiments, the compound preferentially binds TLR7 over other TLR. In embodiments, the compound specifically binds TLR7. In embodiments, the compound binds TLR8. In embodiments, the compound preferentially binds TLR8 over other TLR. In embodiments, the compound specifically binds TLR8. In embodiments, the compound binds TLR9. In embodiments, the compound preferentially binds TLR9 over other TLR. In embodiments, the compound specifically binds TLR9. In embodiments, the compound includes CpG, wherein C and G are nucleotides connected by a phosphodiester internucleotide linkage or phosphodiester derivative internucleotide linkage. In embodiments, the compound includes CpG, wherein C and G are nucleotides connected by a phosphodiester internucleotide linkage. In embodiments, the compound includes CpG, wherein C and G are nucleotides connected by a phosphodiester derivative internucleotide linkage. In embodiments, the CpG is unmethylated.

In embodiments, the compound enters a cell following administration (e.g. to a patient, to the blood stream of a patient, or to the extracellular milieu of the cell) in about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 minutes, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours. In embodiments, the compound enters a cell following administration (e.g. to a patient, to the blood stream of a patient, or to the extracellular milieu of the cell using, for example, oral, suppository, topical, intravenous, parenteral, transmucosal (e.g., buccal, sublingual, palatal, gingival, nasal, vaginal, rectal, or transdermal), intraperitoneal, intramuscular, intralesional, intrathecal, intracranial, intranasal, or subcutaneous administration) in less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 minutes, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours. In embodiments, the compound enters a cell following administration (e.g. to a patient, to the blood stream of a patient, or to the extracellular milieu of the cell using, for example, oral, suppository, topical, intravenous, parenteral, transmucosal (e.g., buccal, sublingual, palatal, gingival, nasal, vaginal, rectal, or transdermal), intraperitoneal, intramuscular, intralesional, intrathecal, intracranial, intranasal, or subcutaneous administration) without co-administration of an agent to facilitate transfection (e.g. an agent with the sole purpose of assisting the compound to enter a cell). In embodiments, the cell is a plasmacytoid dendritic cell, myeloid dendritic cell, myeloid-derived suppressor cell, granulocytic myeloid-derived suppressor cell, macrophage, B cell, activated NK cell, or activated neutrophil. In embodiments, the cell is in the brain, an organ, bone, or bone marrow of a subject. In embodiments, the cell is a plasmacytoid dendritic cell. In embodiments, the cell is a myeloid dendritic cell. In embodiments, the cell is a myeloid-derived suppressor cell. In embodiments, the cell is a granulocytic myeloid-derived suppressor cell. In embodiments, the cell is a macrophage. In embodiments, the cell is a B cell. In embodiments, the cell is an activated NK cell. In embodiments, the cell is an activated neutrophil. In embodiments, the plasmacytoid dendritic cell is in a tumor. In embodiments, the myeloid dendritic cell is in a tumor. In embodiments, the myeloid-derived suppressor cell is in a tumor. In embodiments, the granulocytic myeloid-derived suppressor cell is in a tumor. In embodiments, the macrophage is in a tumor. In embodiments, the B cell is in a tumor. In embodiments, the activated NK cell is in a tumor. In embodiments, the activated neutrophil is in a tumor. In embodiments, the cell is in the brain, an organ, bone, or bone marrow of a subject. In embodiments, the cell is in the brain of a subject. In embodiments, the cell is in an organ of a subject. In embodiments, the cell is in the bone of a subject. In embodiments, the cell is in the bone marrow of a subject.

In embodiments, the compound is not degraded (e.g. in a patient, in the blood stream, at the site of administration, or in the extracellular milieu) for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 minutes, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours. In embodiments, the compound is not degraded (e.g. in a patient, in the blood stream, at the site of administration, or in the extracellular milieu) for an average of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 minutes, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours. In embodiments, the compound is not degraded (e.g. in a patient, in the blood stream, at the site of administration, or in the extracellular milieu) for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 minutes, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours.

In embodiments, the compound has a half-life (e.g. in a patient, in the blood stream, at the site of administration, or in the extracellular milieu) of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 minutes, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours. In embodiments, the compound has a half-life (e.g. in a patient, in the blood stream, at the site of administration, or in the extracellular milieu) of an average of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 minutes, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours. In embodiments, the compound has a half-life (e.g. in a patient, in the blood stream, at the site of administration, or in the extracellular milieu) of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 minutes, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours.

In embodiments, the second nucleic acid sequence includes a Class A CpG DNA sequence, Class B CpG DNA sequence, or Class C CpG DNA sequence. In embodiments, the second nucleic acid sequence includes a Class A CpG DNA sequence. In embodiments, the second nucleic acid sequence includes Class B CpG DNA sequence. In embodiments, the second nucleic acid sequence includes Class C CpG DNA sequence. In embodiments, the second nucleic acid sequence is a Class A CpG DNA sequence, Class B CpG DNA sequence, or Class C CpG DNA sequence. In embodiments, the second nucleic acid sequence is a Class A CpG DNA sequence. In embodiments, the second nucleic acid sequence is Class B CpG DNA sequence. In embodiments, the second nucleic acid sequence is Class C CpG DNA sequence. In embodiments, the second nucleic acid sequence is a sequence described herein. In embodiments, the first nucleic acid sequence is a sequence described herein.

In embodiments, the TLR9-binding DNA substituent includes a CpG motif. In embodiments, the TLR9-binding DNA substituent includes an unmethylated CpG motif. In embodiments, the TLR9-binding DNA substituent includes a CpG motif wherein the CpG is not methylated. In embodiments, the TLR9-binding DNA substituent includes a DNA sequence capable of forming a G-quadruplex. In embodiments, the TLR9-binding DNA substituent includes a Class A CpG DNA sequence. In embodiments, the TLR9-binding DNA substituent includes a Class B CpG DNA sequence. In embodiments, the TLR9-binding DNA substituent includes a C-type CpG DNA sequence.

In embodiments, the TLR-binding DNA substituent binds TLR9. In embodiments, the TLR-binding DNA substituent preferentially binds TLR9 over other TLR. In embodiments, the TLR-binding DNA substituent specifically binds TLR9. In embodiments, the TLR-binding DNA substituent includes CpG, wherein C and G are nucleotides connected by a phosphodiester internucleotide linkage or phosphodiester derivative internucleotide linkage. In embodiments, the compound includes CpG, wherein C and G are nucleotides connected by a phosphodiester internucleotide linkage. In embodiments, the compound includes CpG, wherein C and G are nucleotides connected by a phosphodiester derivative internucleotide linkage. In embodiments, the CpG is unmethylated.

In embodiments, the compound includes a phosphodiester derivative linkage (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite linkages). In embodiments, the compound includes a plurality of phosphodiester derivative linkages (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, O-methylphosphoroamidite linkages, or combinations thereof). In embodiments, the compound includes a plurality of phosphodiester derivative linkages (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, O-methylphosphoroamidite linkages, or combinations thereof) in the first nucleic acid sequence or the second nucleic acid sequence. In embodiments, the compound includes a phosphodiester derivative linkage (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite linkages) in the second nucleic acid sequence (e.g., nucleic acid sequence capable of binding a Toll-like receptor (TLR) protein). In embodiments, the compound includes a phosphodiester derivative linkage (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite linkages) in the second nucleic acid sequence (e.g., nucleic acid sequence capable of binding a Toll-like receptor (TLR) protein) (e.g. endosomal TLR-, TLR3-, TLR7-, TLR8-, or TLR9-binding nucleic acid) substituent. In embodiments, the compound includes a phosphodiester derivative linkage (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite linkages) in the first nucleic acid sequence (e.g., nucleic acid sequence capable of binding to Nuclear Factor Kappa-Light-Chain-Enhancer of Activated B Cells (NF-κB)). In embodiments, the compound includes a phosphodiester derivative linkage (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite linkages) in the first nucleic acid sequence (e.g., nucleic acid sequence capable of binding to Nuclear Factor Kappa-Light-Chain-Enhancer of Activated B Cells (NF-κB)). In embodiments, one or more of the nucleic acid internucleotide linkages in the compound is a phosphodiester derivative linkage (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite linkages), (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphodiester derivative linkages (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, O-methylphosphoroamidite linkages, or combinations thereof)). In embodiments, the compound includes a phosphodiester derivative linkage (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite linkages) in the nucleic acid sequence capable of binding to Nuclear Factor Kappa-Light-Chain-Enhancer of Activated B Cells (NF-κB). In embodiments, one or more of the DNA internucleotide linkages in the compound is a phosphodiester derivative linkage (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite linkages), (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphodiester derivative linkages (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, O-methylphosphoroamidite linkages, or combinations thereof)).

In embodiments, the compound includes a phosphorothioate linkage. In embodiments, the compound includes a plurality of phosphorothioate linkages. In embodiments, the compound includes a plurality of phosphorothioate linkages in the first nucleic acid sequence or the second nucleic acid sequence. In embodiments, the compound includes a phosphorothioate linkage in the second nucleic acid sequence capable of binding a Toll-like receptor (TLR) protein (e.g., TLR9). In embodiments, the compound includes a phosphorothioate linkage in the first nucleic acid sequence capable of binding to Nuclear Factor Kappa-Light-Chain-Enhancer of Activated B Cells (NF-κB). In embodiments, one or more of the nucleic acid internucleotide linkages in the compound is a phosphorothioate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphorothioate linkages). In embodiments, the compound includes a phosphorothioate linkage in the nucleic acid sequence capable of binding to Nuclear Factor Kappa-Light-Chain-Enhancer of Activated B Cells (NF-κB). In embodiments, one or more of the DNA internucleotide linkages in the compound is a phosphorothioate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphorothioate linkages).

In embodiments, the compound includes a phosphoramidate linkage. In embodiments, the compound includes a plurality of phosphoramidate linkages. In embodiments, the compound includes a phosphoramidate linkage in the second nucleic acid sequence capable of binding a Toll-like receptor (TLR) protein (e.g., TLR9). In embodiments, the compound includes a phosphoramidate linkage in the first nucleic acid sequence capable of binding to Nuclear Factor Kappa-Light-Chain-Enhancer of Activated B Cells (NF-κB). In embodiments, one or more of the nucleic acid internucleotide linkages in the compound is a phosphoramidate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphoramidate linkages). In embodiments, the compound includes a phosphoramidate linkage in the nucleic acid sequence capable of binding to Nuclear Factor Kappa-Light-Chain-Enhancer of Activated B Cells (NF-κB). In embodiments, one or more of the DNA internucleotide linkages in the compound is a phosphoramidate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphoramidate linkages).

In embodiments, the compound includes a phosphorodiamidate linkage. In embodiments, the compound includes a plurality of phosphorodiamidate linkages. In embodiments, the compound includes a phosphorodiamidate linkage in the second nucleic acid sequence capable of binding a Toll-like receptor (TLR) protein (e.g., TLR9). In embodiments, the compound includes a phosphorodiamidate linkage in the first nucleic acid sequence capable of binding to Nuclear Factor Kappa-Light-Chain-Enhancer of Activated B Cells (NF-κB). In embodiments, one or more of the nucleic acid internucleotide linkages in the compound is a phosphorodiamidate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphorodiamidate linkages). In embodiments, the compound includes a phosphorodiamidate linkage in the nucleic acid sequence capable of binding to Nuclear Factor Kappa-Light-Chain-Enhancer of Activated B Cells (NF-κB). In embodiments, one or more of the DNA internucleotide linkages in the compound is a phosphorodiamidate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphorodiamidate linkages).

In embodiments, the compound includes a phosphorodithioate linkage. In embodiments, the compound includes a plurality of phosphorodithioate linkages. In embodiments, the compound includes a phosphorodithioate linkage in the second nucleic acid sequence capable of binding a Toll-like receptor (TLR) protein (e.g., TLR9). In embodiments, the compound includes a phosphorodithioate linkage in the first nucleic acid sequence capable of binding to Nuclear Factor Kappa-Light-Chain-Enhancer of Activated B Cells (NF-κB). In embodiments, one or more of the nucleic acid internucleotide linkages in the compound is a phosphorodithioate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphorodithioate linkages). In embodiments, the compound includes a phosphorodithioate linkage in the nucleic acid sequence capable of binding to Nuclear Factor Kappa-Light-Chain-Enhancer of Activated B Cells (NF-κB). In embodiments, one or more of the DNA internucleotide linkages in the compound is a phosphorodithioate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphorodithioate linkages).

In embodiments, the compound includes a phosphonocarboxylic linkage. In embodiments, the compound includes a plurality of phosphonocarboxylic linkages. In embodiments, the compound includes a phosphonocarboxylic linkage in the second nucleic acid sequence capable of binding a Toll-like receptor (TLR) protein (e.g., TLR9). In embodiments, the compound includes a phosphonocarboxylic linkage in the first nucleic acid sequence capable of binding to Nuclear Factor Kappa-Light-Chain-Enhancer of Activated B Cells (NF-κB). In embodiments, one or more of the nucleic acid internucleotide linkages in the compound is a phosphonocarboxylic linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphonocarboxylic linkages). In embodiments, the compound includes a phosphonocarboxylic linkage in the nucleic acid sequence capable of binding to Nuclear Factor Kappa-Light-Chain-Enhancer of Activated B Cells (NF-κB). In embodiments, one or more of the DNA internucleotide linkages in the compound is a phosphonocarboxylic linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphonocarboxylic linkages).

In embodiments, the compound includes a phosphonocarboxylate linkage. In embodiments, the compound includes a plurality of phosphonocarboxylate linkages. In embodiments, the compound includes a phosphonocarboxylate linkage in the second nucleic acid sequence capable of binding a Toll-like receptor (TLR) protein (e.g., TLR9). In embodiments, the compound includes a phosphonocarboxylate linkage in the first nucleic acid sequence capable of binding to Nuclear Factor Kappa-Light-Chain-Enhancer of Activated B Cells (NF-κB). In embodiments, one or more of the nucleic acid internucleotide linkages in the compound is a phosphonocarboxylate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphonocarboxylate linkages). In embodiments, the compound includes a phosphonocarboxylate linkage in the nucleic acid sequence capable of binding to Nuclear Factor Kappa-Light-Chain-Enhancer of Activated B Cells (NF-κB). In embodiments, one or more of the DNA internucleotide linkages in the compound is a phosphonocarboxylate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphonocarboxylate linkages).

In embodiments, the compound includes a phosphonoacetic linkage. In embodiments, the compound includes a plurality of phosphonoacetic linkages. In embodiments, the compound includes a phosphonoacetic linkage in the second nucleic acid sequence capable of binding a Toll-like receptor (TLR) protein (e.g., TLR9). In embodiments, the compound includes a phosphonoacetic linkage in the first nucleic acid sequence capable of binding to Nuclear Factor Kappa-Light-Chain-Enhancer of Activated B Cells (NF-κB). In embodiments, one or more of the nucleic acid internucleotide linkages in the compound is a phosphonoacetic linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphonoacetic linkages). In embodiments, the compound includes a phosphonoacetic linkage in the nucleic acid sequence capable of binding to Nuclear Factor Kappa-Light-Chain-Enhancer of Activated B Cells (NF-κB). In embodiments, one or more of the DNA internucleotide linkages in the compound is a phosphonoacetic linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphonoacetic linkages).

In embodiments, the compound includes a phosphonoformic linkage. In embodiments, the compound includes a plurality of phosphonoformic linkages. In embodiments, the compound includes a phosphonoformic linkage in the second nucleic acid sequence capable of binding a Toll-like receptor (TLR) protein (e.g., TLR9). In embodiments, the compound includes a phosphonoformic linkage in the first nucleic acid sequence capable of binding to Nuclear Factor Kappa-Light-Chain-Enhancer of Activated B Cells (NF-κB). In embodiments, one or more of the nucleic acid internucleotide linkages in the compound is a phosphonoformic linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphonoformic linkages). In embodiments, the compound includes a phosphonoformic linkage in the nucleic acid sequence capable of binding to Nuclear Factor Kappa-Light-Chain-Enhancer of Activated B Cells (NF-κB). In embodiments, one or more of the DNA internucleotide linkages in the compound is a phosphonoformic linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are phosphonoformic linkages).

In embodiments, the compound includes a methyl phosphonate linkage. In embodiments, the compound includes a plurality of methyl phosphonate linkages. In embodiments, the compound includes a methyl phosphonate linkage in the second nucleic acid sequence capable of binding a Toll-like receptor (TLR) protein (e.g., TLR9). In embodiments, the compound includes a methyl phosphonate linkage in the first nucleic acid sequence capable of binding to Nuclear Factor Kappa-Light-Chain-Enhancer of Activated B Cells (NF-κB). In embodiments, one or more of the nucleic acid internucleotide linkages in the compound is a methyl phosphonate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are methyl phosphonate linkages). In embodiments, the compound includes a methyl phosphonate linkage in the nucleic acid sequence capable of binding to Nuclear Factor Kappa-Light-Chain-Enhancer of Activated B Cells (NF-κB). In embodiments, one or more of the DNA internucleotide linkages in the compound is a methyl phosphonate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are methyl phosphonate linkages).

In embodiments, the compound includes a boron phosphonate linkage. In embodiments, the compound includes a plurality of boron phosphonate linkages. In embodiments, the compound includes a boron phosphonate linkage in the second nucleic acid sequence capable of binding a Toll-like receptor (TLR) protein (e.g., TLR9). In embodiments, the compound includes a boron phosphonate linkage in the first nucleic acid sequence capable of binding to Nuclear Factor Kappa-Light-Chain-Enhancer of Activated B Cells (NF-κB). In embodiments, one or more of the nucleic acid internucleotide linkages in the compound is a boron phosphonate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are boron phosphonate linkages). In embodiments, the compound includes a boron phosphonate linkage in the nucleic acid sequence capable of binding to Nuclear Factor Kappa-Light-Chain-Enhancer of Activated B Cells (NF-κB). In embodiments, one or more of the DNA internucleotide linkages in the compound is a boron phosphonate linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are boron phosphonate linkages).

In embodiments, the compound includes a O-methylphosphoroamidite linkage. In embodiments, the compound includes a plurality of O-methylphosphoroamidite linkages. In embodiments, the compound includes a O-methylphosphoroamidite linkage in the second nucleic acid sequence capable of binding a Toll-like receptor (TLR) protein (e.g., TLR9). In embodiments, the compound includes a O-methylphosphoroamidite linkage in the first nucleic acid sequence capable of binding to Nuclear Factor Kappa-Light-Chain-Enhancer of Activated B Cells (NF-κB). In embodiments, one or more of the nucleic acid internucleotide linkages in the compound is a O-methylphosphoroamidite linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are O-methylphosphoroamidite linkages). In embodiments, the compound includes a O-methylphosphoroamidite linkage in the nucleic acid sequence capable of binding to Nuclear Factor Kappa-Light-Chain-Enhancer of Activated B Cells (NF-κB). In embodiments, one or more of the DNA internucleotide linkages in the compound is a O-methylphosphoroamidite linkage (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or all internucleotide linkages in the compound are O-methylphosphoroamidite linkages).

In embodiments, the compound further includes a phosphorothioate linkage in the first nucleic acid sequence or the second nucleic acid sequence. In embodiments, the compound further includes a plurality of phosphorothioate linkages (e.g., a plurality of phosphorothioate linkages in the first nucleic acid sequence or a plurality of phosphorothioate linkages in the second nucleic acid sequence). In embodiments, the compound further includes a phosphorothioate linkage in the first nucleic acid sequence. In embodiments, the compound further a phosphorothioate linkage in the second nucleic acid sequence. In embodiments the nucleic acid sequence (e.g., first or second nucleic acid sequence) is a nucleic acid sequence as described herein.

In embodiments, z1 is 0. In embodiments, z1 is 1. In embodiments, z1 is 2. In embodiments, z1 is 3. In embodiments, z1 is 4. In embodiments, z1 is 5. In embodiments, z1 is 6. In embodiments, z1 is 7. In embodiments, z1 is 8. In embodiments, z1 is 9. In embodiments, z1 is 10. In embodiments, z1 is 11. In embodiments, z1 is 12. In embodiments, z1 is 13. In embodiments, z1 is 14. In embodiments, z1 is 15. In embodiments, z1 is 16. In embodiments, z1 is 17. In embodiments, z1 is 18. In embodiments, z1 is 19. In embodiments, z1 is 20. In embodiments, z2 is independently 0. In embodiments, z2 is independently 1. In embodiments, z2 is independently 2. In embodiments, z2 is independently 3. In embodiments, z2 is independently 4. In embodiments, z2 is independently 5. In embodiments, z2 is independently 6. In embodiments, z2 is independently 7. In embodiments, z2 is independently 8. In embodiments, z2 is independently 9. In embodiments, z2 is independently 10. In embodiments, z2 is independently 11. In embodiments, z2 is independently 12. In embodiments, z2 is independently 13. In embodiments, z2 is independently 14. In embodiments, z2 is independently 15. In embodiments, z2 is independently 16. In embodiments, z2 is independently 17. In embodiments, z2 is independently 18. In embodiments, z2 is independently 19. In embodiments, z2 is independently 20. In embodiments, z3 is 0. In embodiments, z3 is 1. In embodiments, z3 is 2. In embodiments, z3 is 3. In embodiments, z3 is 4. In embodiments, z3 is 5. In embodiments, z3 is 6. In embodiments, z3 is 7. In embodiments, z3 is 8. In embodiments, z3 is 9. In embodiments, z3 is 10. In embodiments, z3 is 11. In embodiments, z3 is 12. In embodiments, z3 is 13. In embodiments, z3 is 14. In embodiments, z3 is 15. In embodiments, z3 is 16. In embodiments, z3 is 17. In embodiments, z3 is 18. In embodiments, z3 is 19. In embodiments, z3 is 20. In embodiments, z4 is 0. In embodiments, z4 is 1. In embodiments, z4 is 2. In embodiments, z4 is 3. In embodiments, z4 is 4. In embodiments, z4 is 5. In embodiments, z4 is 6. In embodiments, z4 is 7. In embodiments, z4 is 8. In embodiments, z4 is 9. In embodiments, z4 is 10. In embodiments, z4 is 11. In embodiments, z4 is 12. In embodiments, z4 is 13. In embodiments, z4 is 14. In embodiments, z4 is 15. In embodiments, z4 is 16. In embodiments, z4 is 17. In embodiments, z4 is 18. In embodiments, z4 is 19. In embodiments, z4 is 20.

In embodiments, z5 is 0. In embodiments, z5 is 1. In embodiments, z5 is 2. In embodiments, z5 is 3. In embodiments, z5 is 4. In embodiments, z5 is 5. In embodiments, z5 is 6. In embodiments, z5 is 7. In embodiments, z5 is 8. In embodiments, z5 is 9. In embodiments, z5 is 10. In embodiments, z5 is 11. In embodiments, z5 is 12. In embodiments, z5 is 13. In embodiments, z5 is 14. In embodiments, z5 is 15. In embodiments, z5 is 16. In embodiments, z5 is 17. In embodiments, z5 is 18. In embodiments, z5 is 19. In embodiments, z5 is 20. In embodiments, z6 is independently 0. In embodiments, z6 is independently 1. In embodiments, z6 is independently 2. In embodiments, z6 is independently 3. In embodiments, z6 is independently 4. In embodiments, z6 is independently 5. In embodiments, z6 is independently 6. In embodiments, z6 is independently 7. In embodiments, z6 is independently 8. In embodiments, z6 is independently 9. In embodiments, z6 is independently 10. In embodiments, z6 is independently 11. In embodiments, z6 is independently 12. In embodiments, z6 is independently 13. In embodiments, z6 is independently 14. In embodiments, z6 is independently 15. In embodiments, z6 is independently 16. In embodiments, z6 is independently 17. In embodiments, z6 is independently 18. In embodiments, z6 is independently 19. In embodiments, z6 is independently 20. In embodiments, z7 is 0. In embodiments, z7 is 1. In embodiments, z7 is 2. In embodiments, z7 is 3. In embodiments, z7 is 4. In embodiments, z7 is 5. In embodiments, z7 is 6. In embodiments, z7 is 7. In embodiments, z7 is 8. In embodiments, z7 is 9. In embodiments, z7 is 10. In embodiments, z7 is 11. In embodiments, z7 is 12. In embodiments, z7 is 13. In embodiments, z7 is 14. In embodiments, z7 is 15. In embodiments, z7 is 16. In embodiments, z7 is 17. In embodiments, z7 is 18. In embodiments, z7 is 19. In embodiments, z7 is 20. In embodiments, z8 is 0. In embodiments, z8 is 1. In embodiments, z8 is 2. In embodiments, z8 is 3. In embodiments, z8 is 4. In embodiments, z8 is 5. In embodiments, z8 is 6. In embodiments, z8 is 7. In embodiments, z8 is 8. In embodiments, z8 is 9. In embodiments, z8 is 10. In embodiments, z8 is 11. In embodiments, z8 is 12. In embodiments, z8 is 13. In embodiments, z8 is 14. In embodiments, z8 is 15. In embodiments, z8 is 16. In embodiments, z8 is 17. In embodiments, z8 is 18. In embodiments, z8 is 19. In embodiments, z8 is 20.

In embodiments, z9 is 0. In embodiments, z9 is 1. In embodiments, z9 is 2. In embodiments, z9 is 3. In embodiments, z9 is 4. In embodiments, z9 is 5. In embodiments, z9 is 6. In embodiments, z9 is 7. In embodiments, z9 is 8. In embodiments, z9 is 9. In embodiments, z9 is 10. In embodiments, z9 is 11. In embodiments, z9 is 12. In embodiments, z9 is 13. In embodiments, z9 is 14. In embodiments, z9 is 15. In embodiments, z9 is 16. In embodiments, z9 is 17. In embodiments, z9 is 18. In embodiments, z9 is 19. In embodiments, z9 is 20. In embodiments, z10 is independently 0. In embodiments, z10 is independently 1. In embodiments, z10 is independently 2. In embodiments, z10 is independently 3. In embodiments, z10 is independently 4. In embodiments, z10 is independently 5. In embodiments, z10 is independently 6. In embodiments, z10 is independently 7. In embodiments, z10 is independently 8. In embodiments, z10 is independently 9. In embodiments, z10 is independently 10. In embodiments, z10 is independently 11. In embodiments, z10 is independently 12. In embodiments, z10 is independently 13. In embodiments, z10 is independently 14. In embodiments, z10 is independently 15. In embodiments, z10 is independently 16. In embodiments, z10 is independently 17. In embodiments, z10 is independently 18. In embodiments, z10 is independently 19. In embodiments, z10 is independently 20. In embodiments, z11 is 0. In embodiments, z11 is 1. In embodiments, z11 is 2. In embodiments, z11 is 3. In embodiments, z11 is 4. In embodiments, z11 is 5. In embodiments, z11 is 6. In embodiments, z11 is 7. In embodiments, z11 is 8. In embodiments, z11 is 9. In embodiments, z11 is 10. In embodiments, z11 is 11. In embodiments, z11 is 12. In embodiments, z11 is 13. In embodiments, z11 is 14. In embodiments, z11 is 15. In embodiments, z11 is 16. In embodiments, z11 is 17. In embodiments, z11 is 18. In embodiments, z11 is 19. In embodiments, z11 is 20. In embodiments, z12 is 0. In embodiments, z12 is 1. In embodiments, z12 is 2. In embodiments, z12 is 3. In embodiments, z12 is 4. In embodiments, z12 is 5. In embodiments, z12 is 6. In embodiments, z12 is 7. In embodiments, z12 is 8. In embodiments, z12 is 9. In embodiments, z12 is 10. In embodiments, z12 is 11. In embodiments, z12 is 12. In embodiments, z12 is 13. In embodiments, z12 is 14. In embodiments, z12 is 15. In embodiments, z12 is 16. In embodiments, z12 is 17. In embodiments, z12 is 18. In embodiments, z12 is 19. In embodiments, z12 is 20.

In embodiments, z13 is 0. In embodiments, z13 is 1. In embodiments, z13 is 2. In embodiments, z13 is 3. In embodiments, z13 is 4. In embodiments, z13 is 5. In embodiments, z13 is 6. In embodiments, z13 is 7. In embodiments, z13 is 8. In embodiments, z13 is 9. In embodiments, z13 is 10. In embodiments, z13 is 11. In embodiments, z13 is 12. In embodiments, z13 is 13. In embodiments, z13 is 14. In embodiments, z13 is 15. In embodiments, z13 is 16. In embodiments, z13 is 17. In embodiments, z13 is 18. In embodiments, z13 is 19. In embodiments, z3 is 20. In embodiments, z14 is independently 0. In embodiments, z14 is independently 1. In embodiments, z14 is independently 2. In embodiments, z14 is independently 3. In embodiments, z14 is independently 4. In embodiments, z14 is independently 5. In embodiments, z14 is independently 6. In embodiments, z14 is independently 7. In embodiments, z14 is independently 8. In embodiments, z14 is independently 9. In embodiments, z14 is independently 10. In embodiments, z14 is independently 11. In embodiments, z14 is independently 12. In embodiments, z14 is independently 13. In embodiments, z14 is independently 14. In embodiments, z14 is independently 15. In embodiments, z14 is independently 16. In embodiments, z14 is independently 17. In embodiments, z14 is independently 18. In embodiments, z14 is independently 19. In embodiments, z14 is independently 20. In embodiments, z15 is 0. In embodiments, z15 is 1. In embodiments, z15 is 2. In embodiments, z15 is 3. In embodiments, z15 is 4. In embodiments, z15 is 5. In embodiments, z15 is 6. In embodiments, z15 is 7. In embodiments, z15 is 8. In embodiments, z15 is 9. In embodiments, z15 is 10. In embodiments, z15 is 11. In embodiments, z15 is 12. In embodiments, z15 is 13. In embodiments, z15 is 14. In embodiments, z15 is 15. In embodiments, z15 is 16. In embodiments, z15 is 17. In embodiments, z15 is 18. In embodiments, z15 is 19. In embodiments, z15 is 20. In embodiments, z16 is 0. In embodiments, z16 is 1. In embodiments, z16 is 2. In embodiments, z16 is 3. In embodiments, z16 is 4. In embodiments, z16 is 5. In embodiments, z16 is 6. In embodiments, z16 is 7. In embodiments, z16 is 8. In embodiments, z16 is 9. In embodiments, z16 is 10. In embodiments, z16 is 11. In embodiments, z16 is 12. In embodiments, z16 is 13. In embodiments, z16 is 14. In embodiments, z16 is 15. In embodiments, z16 is 16. In embodiments, z16 is 17. In embodiments, z16 is 18. In embodiments, z16 is 19. In embodiments, z16 is 20.

An example of a compound as described herein, is shown below. NA1-CS-NA2; wherein NA1 is a first nucleic acid sequence, CS is a covalent spacer, and NA2 is a second nucleic acid sequence. In embodiments, the 5′ terminus of the compound is an OH group. In embodiments, the 5′ terminus of the compound is a phosphate or phosphate derivative group. In embodiments, the 3′ terminus of the compound is an OH group. In embodiments, the 3′ terminus of the compound is a phosphate or phosphate derivative group.

An example of a compound as described herein, is shown below.

NFkB BS1-S1-NFkB BS2-CS-NA2; wherein NFkB BS1 is a first NF-κB binding site nucleic acid sequence, Si is a first spacer, NFkB BS2 is a second NF-κB binding site nucleic acid sequence, CS is a covalent spacer, and NA2 is a second nucleic acid sequence. In embodiments, the 5′ terminus of the compound is an OH group. In embodiments, the 5′ terminus of the compound is a phosphate or phosphate derivative group. In embodiments, the 3′ terminus of the compound is an OH group. In embodiments, the 3′ terminus of the compound is a phosphate or phosphate derivative group.

An example of a compound as described herein, is shown below. NFkB BS1-S1-NFkB BS2-CS-TLR BS1-S2-TLR BS2; wherein NFkB BS1 is a first NF-κB binding site nucleic acid sequence, S1 is a first spacer, NFkB BS2 is a second NF-κB binding site nucleic acid sequence, CS is a covalent spacer, TLR BS1 is a first TLR binding site nucleic acid sequence, S2 is a second spacer, and TLR BS2 is a second TLR binding site nucleic acid sequence. In embodiments, the 5′ terminus of the compound is an OH group. In embodiments, the 5′ terminus of the compound is a phosphate or phosphate derivative group. In embodiments, the 3′ terminus of the compound is an OH group. In embodiments, the 3′ terminus of the compound is a phosphate or phosphate derivative group.

An example of a compound as described herein, is shown below. TM1-S3-NA1-CS-NA2-S4-TM2; wherein TM1 is a first terminal moiety, S3 is a third spacer, NA1 is a first nucleic acid sequence, CS is a covalent spacer, NA2 is a second nucleic acid sequence, S4 is a fourth spacer, and TM2 is a second terminal moiety.

An example of a compound as described herein, is shown below. TM1-S3-NFkB BS1-S1-NFkB BS2-CS-TLR BS1-S2-TLR BS2-S4-TM2; wherein TM1 is a first terminal moiety, S3 is a third spacer, NFkB BS1 is a first NF-κB binding site nucleic acid sequence, S1 is a first spacer, NFkB BS2 is a second NF-κB binding site nucleic acid sequence, CS is a covalent spacer, TLR BS1 is a first TLR binding site nucleic acid sequence, S2 is a second spacer, and TLR BS2 is a second TLR binding site nucleic acid sequence, S4 is a fourth spacer, and TM2 is a second terminal moiety.

In embodiments, each of the components shown above (e.g., TM1, S3, NA1, NA2, NFkB BS1, Si, NFkB BS2, CS, TLR BS1, S2, TLR BS2, S4, and TM2) is connected to another component shown above through a phosphodiester or phosphodiester derivative.

### III. Pharmaceutical Compositions

In an aspect is provided a pharmaceutical composition including a pharmaceutically acceptable excipient and a compound, or pharmaceutically acceptable salt thereof, described herein (including in an aspect, embodiment, table, figure, claim, sequence listing, or example). In embodiments of the pharmaceutical compositions, the compound, or pharmaceutically acceptable salt thereof, as described herein (including in an aspect, embodiment, table, figure, claim, sequence listing, or example), is included in a therapeutically effective amount.

In embodiments, the pharmaceutical composition further includes a second agent (e.g. therapeutic agent). In embodiments, the second agent is an anti-cancer agent. In embodiments, the second agent is an anti-viral agent. In embodiments of the pharmaceutical compositions, the pharmaceutical composition includes a second agent (e.g. therapeutic agent) in a therapeutically effective amount.

### IV. Methods of Use

In an aspect is provided a method of treating a disease, selected from cancer, an infectious disease, an autoimmune disease, and an inflammatory disease in a patient in need of such treatment, the method including administering a therapeutically effective amount of a compound as described herein, including in an aspect, embodiment, table, figure, claim, sequence listing, or example, to the patient.

In an aspect is provided a compound as described herein, for use in treating a disease, selected from cancer, an infectious disease, an autoimmune disease, and an inflammatory disease in a patient in need of such treatment, the use including administering a therapeutically effective amount of the compound to the patient.

In embodiments, the disease is cancer.

In an aspect is provided a method of treating cancer in a patient in need of such treatment, the method including administering a therapeutically effective amount of a compound as described herein, including in an aspect, embodiment, table, figure, claim, sequence listing, or example, to the patient.

In an aspect is provided a compound as described herein, for use in treating cancer in a patient in need of such treatment, the use including administering a therapeutically effective amount of the compound to the patient.

In embodiments, the cancer (e.g. cancer cell) has an increased level of NF-κB (e.g. activity, mRNA, or protein) relative to a control (e.g. non-cancerous cell of the same type as the cancer cell). In embodiments, the cancer has an increased level of TLR9 relative to a control (e.g. non-cancerous cell of the same type as the cancer cell). In embodiments, the cancer has an increased level of TLR (e.g. endosomal TLR, TLR3, TLR7, TLR8, or TLR9) relative to a control (e.g. non-cancerous cell of the same type as the cancer cell). In embodiments, the NF-κB is human.

In embodiments, cancer is non-Hodgkin's lymphoma. In embodiments, the cancer is B-cell lymphoma (BCL) or Mantle cell lymphoma (MCL). In embodiments, the cancer is B-cell lymphoma (BCL). In embodiments, the cancer is Mantle cell lymphoma (MCL). In embodiments, the cancer is Diffuse large B-cell lymphoma (DLBCL), activated B-cell subtype Diffuse large B-cell lymphoma (ABC-DBLCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), Chronic lymphocytic leukemia (CLL), or acute lymphoblastic leukemia (ALL). In embodiments, the cancer is Diffuse large B-cell lymphoma (DLBCL). In embodiments, the cancer is activated B-cell subtype Diffuse large B-cell lymphoma (ABC-DBLCL). In embodiments, the cancer is germinal center B-cell like diffuse large B-cell lymphoma. In embodiments, the cancer is Follicular lymphoma. In embodiments, the cancer is Marginal zone B-cell lymphoma (MZL). In embodiments, the cancer is Mucosa-Associated Lymphatic Tissue lymphoma (MALT). In embodiments, the cancer is chronic myeloid leukemia (CML). In embodiments, the cancer is acute myeloid leukemia (AML). In embodiments, the cancer is myelodysplastic syndromes (MDS). In embodiments, the cancer is Chronic lymphocytic leukemia (CLL). In embodiments, the cancer is acute lymphoblastic leukemia (ALL). In embodiments, the cancer is relapsed or resistant B-cell lymphoma (BCL). In embodiments, the cancer is relapsed B-cell lymphoma (BCL). In embodiments, the cancer is resistant B-cell lymphoma (BCL). In embodiments, the cancer is primary mediastinal (thymic) large B cell lymphoma. In embodiments, the cancer is T cell/histiocyte-rich large B-cell lymphoma. In embodiments, the cancer is Primary cutaneous diffuse large B-cell lymphoma, leg type (Primary cutaneous DLBCL, leg type). In embodiments, the cancer is EBV positive diffuse large B-cell lymphoma of the elderly. In embodiments, the cancer is Diffuse large B-cell lymphoma associated with inflammation. In embodiments, the cancer is Burkitt's lymphoma. In embodiments, the cancer is lymphoplasmacytic lymphoma, which may manifest as Waldenstrom's macroglobulinemia. In embodiments, the cancer is Nodal marginal zone B cell lymphoma (NMZL). In embodiments, the cancer is splenic marginal zone lymphoma (SMZL). In embodiments, the cancer is intravascular large B-cell lymphoma. In embodiments, the cancer is Primary effusion lymphoma. In embodiments, the cancer is Lymphomatoid granulomatosis. In embodiments, the cancer is Primary central nervous system lymphoma. In embodiments, the cancer is ALK-positive large B-cell lymphoma. In embodiments, the cancer is Plasmablastic lymphoma. In embodiments, the cancer is double-hit lymphoma. In embodiments, the cancer is double-expressor lymphoma.

In embodiments, the cancer is head and neck squamous cell carcinoma (HNSCC). In embodiments, the cancer is breast cancer. In embodiments, the cancer is glioma.

In an aspect is provided a method of inhibiting the growth of a cancer cell including contacting the cancer cell with a compound described herein (including in an aspect, embodiment, table, figure, claim, sequence listing, or example). In embodiments, the cell forms part of an organism. In embodiments, the organism is a mammal.

In embodiments, the cancer cell includes a level of TLR (e.g. endosomal TLR, TLR3, TLR7, TLR8, or TLR9) greater than a non-cancerous cell control. In embodiments, the cancer cell includes a level of TLR9 greater than a non-cancerous cell control. In embodiments, the TLR is an endosomal TLR. In embodiments, the TLR is TLR3. In embodiments, the TLR is TLR7. In embodiments, the TLR is TLR8. In embodiments, the TLR is TLR9. In embodiments, the method or use includes inducing apoptosis of the cancer cell. In embodiments, the method or use includes inducing apoptosis in a cancer cell but not a non-cancerous cell. In embodiments, the method or use includes inducing apoptosis in a cancer cell in a patient but not a non-cancerous cell in the same patient. In embodiments, the method or use includes inducing apoptosis in a cancer cell but not a non-cancer cell of the same cell type as the cancer cell (e.g. lung cell, breast cell, pancreatic cell, colorectal cell, prostate cell, hematopoietic cell). In embodiments, the cancer cell is in the brain. In embodiments, the cancer cell is in an organ. In embodiments, the cancer cell is in a bone. In embodiments, the cancer cell is in bone marrow.

In embodiments, the method further includes administering an anti-cancer agent, radiation, or a combination thereof. In embodiments, the method further includes administering an anti-cancer agent. In embodiments, the method further includes administering radiation. In embodiments, the method further includes administering an anticancer agent and radiation. In embodiments, the method further includes administering tisagenlecleucel (Kymriah™) or axicabtagene ciloleucel (Yescarta®). In embodiments, the method further includes administering tisagenlecleucel (Kymriah™). In embodiments, the method further includes administering axicabtagene ciloleucel (Yescarta®).

In embodiments, the anti-cancer agent is an immune checkpoint inhibitor.

In embodiments, the immune checkpoint inhibitor is a PD-1 inhibitor, PD-L1 inhibitor, or CTLA-4 inhibitor. In embodiments, the immune checkpoint inhibitor is a PD-1 inhibitor. In embodiments, the immune checkpoint inhibitor is a PD-L1 inhibitor. In embodiments, the immune checkpoint inhibitor is a CTLA-4 inhibitor. In embodiments, the PD-1 inhibitor is pembrolizumab. In embodiments, the PD-1 inhibitor is nivolumab. In embodiments, the PD-1 inhibitor is spartalizumab (PDR001). In embodiments, the PD-1 inhibitor is cemiplimab. In embodiments, the PD-L1 inhibitor is atezolizumab. In embodiments, the PD-L1 inhibitor is avelumab. In embodiments, the PD-L1 inhibitor is durvalumab. In embodiments, the CTLA-4 inhibitor is ipilimumab. In embodiments, the CTLA-4 inhibitor is tremelimumab.

In embodiments, the immune checkpoint inhibitor is pembrolizumab, nivolumab, spartalizumab (PDR001), cemiplimab, AMP-224, AMP-514, PDR001, atezolizumab, avelumab, durvalumab, BMX-936559, CK-301, ipilimumab, or tremelimumab. In embodiments, the immune checkpoint inhibitor is pembrolizumab. In embodiments, the immune checkpoint inhibitor is nivolumab. In embodiments, the immune checkpoint inhibitor is spartalizumab (PDR001). In embodiments, the immune checkpoint inhibitor is cemiplimab. In embodiments, the immune checkpoint inhibitor is AMP-224. In embodiments, the immune checkpoint inhibitor is AMP-514. In embodiments, the immune checkpoint inhibitor is PDR001. In embodiments, the immune checkpoint inhibitor is atezolizumab. In embodiments, the immune checkpoint inhibitor is avelumab. In embodiments, the immune checkpoint inhibitor is durvalumab. In embodiments, the immune checkpoint inhibitor is BMX-936559. In embodiments, the immune checkpoint inhibitor is CK-301. In embodiments, the immune checkpoint inhibitor is ipilimumab. In embodiments, the immune checkpoint inhibitor is tremelimumab.

In embodiments, the anti-cancer agent is a chimeric antigen receptor T cell. In embodiments, the chimeric antigen receptor T cell is autologous. In embodiments, the chimeric antigen receptor T cell is allogeneic. In embodiments, the chimeric antigen receptor T cell binds CD19. In embodiments, the chimeric antigen receptor T cell is tisagenlecleucel. In embodiments, the chimeric antigen receptor T cell is axicabtagene ciloleucel.

In an aspect is provided a method of treating graft-versus-host disease in a patient in need of such treatment, the method including administering a therapeutically effective amount of a compound as described herein to the patient. In embodiments, the patient received a tissue transplant. In embodiments, the patient received an allotransplantation. In embodiments, the patient received allogeneic Hematopoietic cell transplantation (allo-HCT). In embodiments, the graft-versus-host disease is acute graft-versus-host disease.

In an aspect is provided a method of reducing the level of PD-1 protein in a cell, the method including contacting the cell with a compound as described herein. In embodiments, the cell is a T cell. In embodiments, the T cell is an adaptive T cell. In embodiments, the T cell is a CD8+ T cell. In embodiments, the T cell is a CD4+ T cell. In embodiments, the T cell is a helper CD4+ T cell. In embodiments, the T cell is a cytotoxic CD8+ T cell. In embodiments, the T cell is a memory T cell. In embodiments, the T cell is a regulatory CD4+ T cell. In embodiments, the T cell is an innate-like T cell. In embodiments, the T cell is a natural killer T cell. In embodiments, the T cell is a mucosal associated invariant. In embodiments, the T cell is a gamma delta T cell. In embodiments, the cell is a B cell. In embodiments, the B cell is a Plasmablast. In embodiments, the B cell is a Lymphoplasmacytoid cell. In embodiments, the B cell is a memory B cell. In embodiments, the B cell is a follicular (FO) B cell. In embodiments, the B cell is a marginal zone (MZ) B cell. In embodiments, the B cell is a B-1 cell. In embodiments, the B cell is a regulatory B (Breg) cell.

In an aspect is provided a method of reducing the level of PD-L1 protein in a cell, the method including contacting the cell with a compound as described herein. In embodiments, the cell is a CD11b+ cell. In embodiments, the cell is a microphage.

In an aspect is provided a method of treating an autoimmune disease in a patient in need of such treatment, the method including administering a therapeutically effective amount of a compound as described herein to the patient. In embodiments, the disease is graft-vs-host disease, Systemic lupus erythematosus (SLE), or Crohn's disease.

In an aspect is provided a compound as described herein, including embodiments, for use in treating an autoimmune disorder in a patient in need of such treatment, the use including administering a therapeutically effective amount of the compound to the patient.

In embodiments, the method or use includes systemic administration of the compound. In embodiments, the method or use includes parenteral administration of the compound. In embodiments, the method or use includes intravenous administration of the compound. In embodiments, the method or use includes administration directly to a tumor.

In an aspect is provided a method of treating an inflammatory disease in a patient in need of such treatment, the method including administering a therapeutically effective amount of a compound as described herein to the patient.

In an aspect is provided a method of treating an infectious disease in a patient in need of such treatment, the method including administering a therapeutically effective amount of a compound as described herein to the patient. In embodiments, the infectious disease is a viral infection (e.g., an RNA viral infection). In embodiments, the infectious disease is a Zika viral infection.

In an aspect is provided a method of treating a viral disease associated with NF-κB-dependent immunosuppression in a patient in need of the treatment, the method including administering a compound, or pharmaceutically acceptable salt thereof, described herein (including in an aspect, embodiment, table, figure, claim, sequence listing, or example).

In embodiments, the viral disease associated with NF-κB-dependent immunosuppression is HHV-1 infection. In embodiments, the viral disease associated with NF-κB-dependent immunosuppression is HHV-2 infection. In embodiments, the viral disease associated with NF-κB-dependent immunosuppression is HHV-3 infection. In embodiments, the viral disease associated with NF-κB-dependent immunosuppression is HHV-4 infection. In embodiments, the viral disease associated with NF-κB-dependent immunosuppression is HHV-5 infection. In embodiments, the viral disease associated with NF-κB-dependent immunosuppression is HHV-6A infection. In embodiments, the viral disease associated with NF-κB-dependent immunosuppression is HHV-6B infection. In embodiments, the viral disease associated with NF-κB-dependent immunosuppression is HHV-7 infection. In embodiments, the viral disease associated with NF-κB-dependent immunosuppression is HHV-8 infection. In embodiments, the viral disease associated with NF-κB-dependent immunosuppression is hepatitis A virus infection. In embodiments, the viral disease associated with NF-κB-dependent immunosuppression is hepatitis B virus infection. In embodiments, the viral disease associated with NF-κB-dependent immunosuppression is hepatitis C virus infection. In embodiments, the viral disease associated with NF-κB-dependent immunosuppression is hepatitis D virus infection. In embodiments, the viral disease associated with NF-κB-dependent immunosuppression is hepatitis E virus infection. In embodiments, the viral disease associated with NF-κB-dependent immunosuppression is HIV infection. In embodiments, the viral disease associated with NF-κB-dependent immunosuppression is ZIKA infection.

In embodiments is provided a method of inhibiting NF-κB in BCL cells and primary myeloid cells. In embodiments is provided a method of inducing apoptosis. In embodiments is provided a method of elevating caspase-3 activity. In embodiments is provided a method of enhancing the apoptotic effect of irradiation. In embodiments is provided a method of enhancing the cytotoxic effect of doxorubicin-based chemotherapy. In embodiments is provided a method of enhancing the cytotoxic effect of bortezomib. In embodiments is provided a method of enhancing the cytotoxic effect of ibrutinib. In embodiments is provided a method of enhancing the effect of rituximab. In embodiments is provided a method of enhancing the effect of anti-OX-40. In embodiments is provided a method of enhancing the effect of anti-CTLA-4 antibody. In embodiments is provided a method of shrinking the size of a tumor (e.g. in cancer). In embodiments is provided a method of treatment combining irradiation and modulation of NF-κB activity.

In embodiments is provided a method of diminishing tumor size both locally, at the treated site, and systemically, at the distant/untreated tumor site. In embodiments is provided a method of selectively suppressing NF-κB in tumor cells to induce immune-mediated anti-tumor effects. In embodiments is provided a method of selectively suppressing NF-κB in tumor-associated myeloid cells to induce immune-mediated anti-tumor effects. In embodiments is provided a method of selectively inhibiting NF-κB signaling in a tumor. In embodiments is provided a method of selectively inhibiting of NF-κB signaling in TME-associated myeloid cells. In embodiments, is a method of inducing JAK/STAT1-mediated Caspase3 cleavage and apoptosis.

In embodiments is provided a method of enhancing T cell infiltration. In embodiments is provided a method of enhancing T cell infiltration, especially the CD4⁺ T cells. In embodiments is provided a method of enhancing T cell infiltration, especially the CD4⁺ T cells but not CD8⁺ T cells. In embodiments, is a method of reducing PD-1 expression. In embodiments, is a method of reducing PD-L1 expression on CD11b⁺ cells in tumors. In embodiments, is a method of generating systemic antitumor immune responses.

### V. Embodiments

Embodiment P1. A compound comprising a first nucleic acid sequence capable of binding to Nuclear Factor Kappa-Light-Chain-Enhancer of Activated B Cells (NF-κB) and a second nucleic acid sequence capable of binding a Toll-like receptor (TLR) protein covalently bound to said first nucleic acid.

Embodiment P2. The compound of embodiment P1, wherein the first nucleic acid sequence capable of binding to NF-κB comprises a first NF-κB binding site nucleic acid sequence and a second NF-κB binding site nucleic acid sequence connected through a spacer, wherein said spacer is a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment P3. The compound of embodiments P1 or P2, wherein the Toll-like receptor protein is human Toll-like receptor 3, Toll-like receptor 7, Toll-like receptor 8, or Toll-like receptor 9.

Embodiment P4. The compound of embodiments P1 or P2, wherein the Toll-like receptor protein is human Toll-like receptor 9.

Embodiment P5. The compound of any one of embodiments P1 to P3, wherein the second nucleic acid sequence comprises a CpG motif, a GpC motif, or a phosphorothioated nucleic acid sequence having at least 10 nucleotides.

Embodiment P6. The compound of any one of embodiments P1 to P3, wherein the second nucleic acid sequence comprises an unmethylated CpG motif.

Embodiment P7. The compound of one of embodiments P1 to P6, wherein the second nucleic acid sequence comprises a Class A CpG DNA sequence, Class B CpG DNA sequence, or Class C CpG DNA sequence.

Embodiment P8. The compound of one of embodiments P1 to P6, wherein the second nucleic acid sequence capable of binding a TLR protein comprises a first TLR binding site nucleic acid sequence and a second TLR site nucleic acid sequence connected through a spacer, wherein said spacer is a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment P9. The compound of any one of embodiments P1 to P8, further comprising a phosphorothioate linkage in the first nucleic acid sequence or the second nucleic acid sequence.

Embodiment P10. The compound of any one of embodiments P1 to P8, further comprising a plurality of phosphorothioate linkages.

Embodiment P11. The compound of any one of embodiments P1 to P8, further comprising a phosphorothioate linkage in the first nucleic acid sequence.

Embodiment P12. The compound of any one of embodiments P1 to P8, further comprising a phosphorothioate linkage in the second nucleic acid sequence.

Embodiment P13. The compound of any one of embodiments P2 to P12, wherein the spacer has the formula:

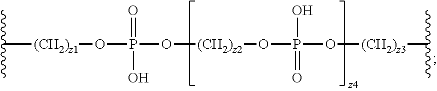

wherein
z1, z2, z3 and z4 are independently integers from 0 to 20.

Embodiment P14. The compound of any one of embodiments P2 to P12, wherein the spacer is a substituted or unsubstituted C₁-C₄₀ alkylene, substituted or unsubstituted 2 to 40 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

Embodiment P15. The compound of any one of embodiments P1 to P14, wherein the compound is covalently bonded to a terminal moiety.

Embodiment P16. The compound of any one of embodiments P1 to P14, wherein the terminal moiety is a hydrogen, monophosphate, polyphosphate, —OH, —NH₂, or

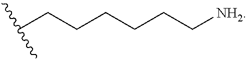 [SMILES: *CCCCCCN]

Embodiment P17. The compound of embodiment P1, having the sequence: SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

Embodiment P18. The compound of embodiment P1, having the sequence: SEQ ID NO: 5.

Embodiment P19. A pharmaceutical composition comprising a pharmaceutically acceptable excipient and the compound of any one of embodiments P1 to P18.

Embodiment P20. A method of treating cancer, an infectious disease, an autoimmune disease, or an inflammatory disease in a patient in need of such treatment, the method comprising administering a therapeutically effective amount of a compound of one of embodiments P1 to P17 to a patient in need thereof.

Embodiment P21. The method of embodiment P20, wherein the cancer is non-Hodgkin's lymphoma.

Embodiment P22. The method of embodiment P20, wherein the cancer is B-cell lymphoma (BCL) or Mantle cell lymphoma (MCL).

Embodiment P23. The method of embodiment P20, wherein the cancer is Diffuse large B-cell lymphoma (DLBCL), Follicular lymphoma, Marginal zone B-cell lymphoma (MZL), Mucosa-Associated Lymphatic Tissue lymphoma (MALT), chronic myeloid leukemia (CML), or myelodysplastic syndromes (MDS), or Chronic lymphocytic leukemia (CLL).

Embodiment P24. The method of embodiment P20, further comprising administering an anti-cancer agent, radiation, or a combination thereof.

Embodiment P25. A method of treating a graft-versus-host disease in a patient in need of such treatment, the method comprising administering a therapeutically effective amount of a compound of one of embodiments P1 to P18 to a patient.

Embodiment P26. The method of embodiment P25, wherein the patient received a tissue transplant.

Embodiment P27. The method of embodiment P25, wherein the patient received an allotransplantation.

Embodiment P28. The method of embodiment P25, wherein the patient received allogeneic Hematopoietic cell transplantation (allo-HCT).

Embodiment P29. The method of embodiment P25, wherein the graft-versus-host disease is acute graft-versus-host disease.

### I. Additional Embodiments

Embodiment 1. A compound comprising a first nucleic acid sequence capable of binding to Nuclear Factor Kappa-Light-Chain-Enhancer of Activated B Cells (NF-κB) and a second nucleic acid sequence capable of binding a Toll-like receptor (TLR) protein, wherein said first nucleic acid sequence and said second nucleic acid sequence are covalently bound through a covalent spacer, wherein said covalent spacer is a bond, a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 2. The compound of embodiment 1, wherein the first nucleic acid sequence capable of binding to NF-κB comprises a first NF-κB binding site nucleic acid sequence and a second NF-κB binding site nucleic acid sequence connected through a first spacer, wherein said first spacer is a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 3. The compound of embodiments 1 or 2, wherein the Toll-like receptor protein is human Toll-like receptor 3, Toll-like receptor 7, Toll-like receptor 8, or Toll-like receptor 9.

Embodiment 4. The compound of embodiments 1 or 2, wherein the Toll-like receptor protein is human Toll-like receptor 9.

Embodiment 5. The compound of any one of embodiments 1 to 3, wherein the second nucleic acid sequence comprises a aliphatic spacer, CpG motif, a GpC motif, or a phosphorothioated nucleic acid having at least 10 nucleotides.

Embodiment 6. The compound of any one of embodiments 1 to 3, wherein the second nucleic acid sequence comprises an unmethylated CpG motif.

Embodiment 7. The compound of any one of embodiments 1 to 6, wherein the second nucleic acid sequence comprises a Class A CpG DNA sequence, Class B CpG DNA sequence, or Class C CpG DNA sequence.

Embodiment 8. The compound of any one of embodiments 1 to 6, wherein the second nucleic acid sequence capable of binding a TLR protein comprises a first TLR binding site nucleic acid sequence and a second TLR site nucleic acid sequence connected through a second spacer, wherein said second spacer is a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 9. The compound of any one of embodiments 1 to 8, further comprising a phosphorothioate linkage in the first nucleic acid sequence or the second nucleic acid sequence.

Embodiment 10. The compound of any one of embodiments 1 to 8, further comprising a plurality of phosphorothioate linkages in the first nucleic acid sequence or the second nucleic acid sequence.

Embodiment 11. The compound of any one of embodiments 1 to 8, further comprising a phosphorothioate linkage in the first nucleic acid sequence.

Embodiment 12. The compound of any one of embodiments 1 to 8, further comprising a phosphorothioate linkage in the second nucleic acid sequence.

Embodiment 13. The compound of any one of embodiments 2 to 12, wherein the first spacer has the formula:

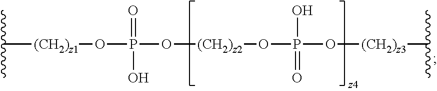

wherein
z1, z2, z3 and z4 are independently integers from 0 to 20.

Embodiment 14. The compound of any one of embodiments 2 to 12, wherein the first spacer is a substituted or unsubstituted C₁-C₄₀ alkylene, substituted or unsubstituted 2 to 40 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

Embodiment 15. The compound of any one of embodiments 1 to 14, wherein the covalent spacer has the formula:

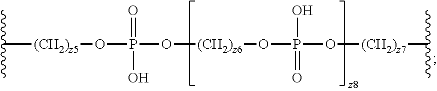

wherein
z5, z6, z7 and z8 are independently integers from 0 to 20.

Embodiment 16. The compound of any one of embodiments 1 to 14, wherein the covalent spacer is a substituted or unsubstituted C₁-C₄₀ alkylene, substituted or unsubstituted 2 to 40 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

Embodiment 17. The compound of any one of embodiments 1 to 16 further comprising a first terminal moiety that is covalently bound through a third spacer to the first nucleic acid sequence.

Embodiment 18. The compound of embodiment 17, wherein the third spacer has the formula:

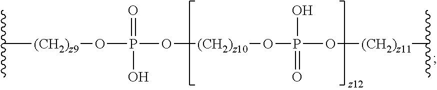

wherein
z9, z10, z11 and z12 are independently integers from 0 to 20.

Embodiment 19. The compound of any one of embodiments 1 to 16, further comprising a second terminal moiety that is covalently bound through a fourth spacer to the second nucleic acid sequence.

Embodiment 20. The compound of embodiment 19, wherein the covalent spacer has the formula:

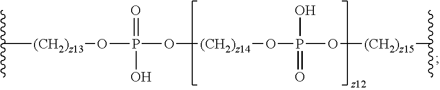

wherein
z13, z14, z15 and z16 are independently integers from 0 to 20.

Embodiment 21. The compound of any one of embodiments 1 to 16, further comprising a first terminal moiety that is covalently bound through a third spacer to the first nucleic acid sequence, and a second terminal moiety that is covalently bound through a fourth spacer to the second nucleic acid sequence.

Embodiment 22. The compound of embodiment 21, wherein the first and second terminal moiety are independently a hydrogen, monophosphate, polyphosphate, —OH, —NH₂,

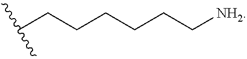 [SMILES: *CCCCCCN]

Embodiment 23. The compound of embodiment 1, comprising the sequence: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, or SEQ ID NO: 21.

Embodiment 24. The compound of embodiment 1, comprising the sequence: SEQ ID NO: 5.

Embodiment 25. A pharmaceutical composition comprising a pharmaceutically acceptable excipient and the compound of any one of embodiments 1 to 24.

Embodiment 26. A method of treating a disease selected from cancer, an infectious disease, an autoimmune disease, and an inflammatory disease in a patient in need of such treatment, the method comprising administering a therapeutically effective amount of a compound of one of embodiments 1 to 24 to a patient in need thereof.

Embodiment 27. The method of embodiment 26, wherein the disease is cancer.

Embodiment 28. The method of embodiment 27, wherein the cancer is non-Hodgkin's lymphoma.

Embodiment 29. The method of embodiment 27, wherein the cancer is B-cell lymphoma (BCL) or Mantle cell lymphoma (MCL).

Embodiment 30. The method of embodiment 27, wherein the cancer is Diffuse large B-cell lymphoma (DLBCL), activated B-cell subtype Diffuse large B-cell lymphoma (ABC-DBLCL), Follicular lymphoma, Marginal zone B-cell lymphoma (MZL), Mucosa-Associated Lymphatic Tissue lymphoma (MALT), chronic myeloid leukemia (CML), acute myeloid leukemia (AML), myelodysplastic syndromes (MDS), Chronic lymphocytic leukemia (CLL), or acute lymphoblastic leukemia (ALL).

Embodiment 31. The method of one of embodiments 27 to 30, further comprising administering an anti-cancer agent, radiation, or a combination thereof.

Embodiment 32. The method of embodiment 31, wherein the anti-cancer agent is an immune checkpoint inhibitor.

Embodiment 33. The method of embodiment 32, wherein the immune checkpoint inhibitor is a PD-1 inhibitor, PD-L1 inhibitor, or CTLA4 inhibitor.

Embodiment 34. The method of embodiment 32, wherein the immune checkpoint inhibitor is pembrolizumab, nivolumab, cemiplimab, AMP-224, AMP-514, PDR001, atezolizumab, avelumab, durvalumab, BMX-936559, CK-301, ipilimumab, or tremelimumab.

Embodiment 35. The method of embodiment 31, wherein the anti-cancer agent is a chimeric antigen receptor T cell.

Embodiment 36. The method of embodiment 35, wherein the chimeric antigen receptor T cell is autologous.

Embodiment 37. The method of embodiment 35, wherein the chimeric antigen receptor T cell is allogeneic.

Embodiment 38. The method of embodiment 35, wherein the chimeric antigen receptor T cell binds CD19.

Embodiment 39. The method of embodiment 35, wherein the chimeric antigen receptor T cell is tisagenlecleucel.

Embodiment 40. The method of embodiment 35, wherein the chimeric antigen receptor T cell is axicabtagene ciloleucel.

Embodiment 41. The method of embodiment 27, further comprising use of tisagenlecleucel or axicabtagene ciloleucel.

Embodiment 42. A method of treating a graft-versus-host disease in a patient in need of such treatment, the method comprising administering a therapeutically effective amount of a compound of one of embodiments 1 to 24 to a patient.

Embodiment 43. The method of embodiment 42, wherein the patient received a tissue transplant.

Embodiment 44. The method of embodiment 42, wherein the patient received an allotransplantation.

Embodiment 45. The method of embodiment 42, wherein the patient received allogeneic Hematopoietic cell transplantation (allo-HCT).

Embodiment 46. The method of embodiment 42, wherein the graft-versus-host disease is acute graft-versus-host disease.

Embodiment 47. A method of reducing the level of PD-1 protein in a cell, the method comprising contacting the cell with a compound of one of embodiments 1 to 24.

Embodiment 48. The method of embodiment 47, wherein the cell is a T cell.

Embodiment 49. The method of embodiment 48, wherein the T cell is a CD8+ T cell.

Embodiment 50. The method of embodiment 48, wherein the T cell is a CD4+ T cell.

Embodiment 51. The method of embodiment 47, wherein the cell is a B cell.

Embodiment 52. A method of reducing the level of PD-L1 protein in a cell, the method comprising contacting the cell with a compound of one of embodiments 1 to 18.

Embodiment 53. The method of embodiment 52, wherein the cell is a CD11b+ cell.

Embodiment 54. The method of one of embodiments 47 to 53, wherein the cell is in a patient.

### Examples

### Example 1: Inhibition of Survival Signaling in B-Cell Lymphoma Using TLR9-Targeted Delivery of NF-κB Decoy Oligodeoxynucleotides In Vitro and In Vivo

Despite of recent advances in treatment of non-Hodgkin B-cell lymphoma (BCL), significant number of patients develops resistance to therapy leading to cancer relapse. NF-κB signaling pathway, which plays critical role in cancer cell survival and proliferation, is known to be partly responsible for treatment resistance in BCL. Despite being an attractive molecular target, transcription factors such as NF-κB are challenging pharmacologic targets. Therefore, we developed an oligonucleotide-based inhibitor of NF-κB, which is based on the conjugation of a double-stranded NF-κB specific DNA sequence (decoy ODN) with CpG motif, which facilitates specific delivery to Toll-like receptor 9 (TLR9) expressing cells, such as B-cell lymphoma. CpG-NF-κB decoy conjugates showed improved uptake in human B-cell and mantle cell lymphoma such as U2932, OCI-Ly3 and RL cells compared to decoy molecules alone. In addition, we confirmed that CpG-NF-κB decoy inhibited NF-κB DNA-binding activity and reduced expression of target genes, such as CCND2 or MYC. The NF-κB inhibition induced dose-dependent cell death in cancer cells and enhanced cytotoxicity induced by irradiation or chemotherapy (e.g., Bortezomib) in vitro. In xenotransplanted models of U2932 B-cell lymphoma, CpG-NF-κB decoy effectively reduced NF-κB activity in whole tumors and significantly inhibited lymphoma progression. These studies suggest that CpG-NF-κB decoy strategy has potential to provide new, clinical relevant approach to reduce therapeutic resistance in B-cell lymphoma.

CpG-NFκB decoy facilitates specific delivery to BCL tumor cells and myeloid cells, prevents nuclear translocation and DNA-binding of NF-κB, inhibits downstream gene expression and results in apoptosis in B-cell lymphoma cells.

Intratumoral injections of CpG-NFκB decoy, alone or in combination with standard therapies, trigger regression of BCL tumors in vivo through inhibition of NF-κB nuclear localization and enhanced cell death.

We demonstrate the feasibility of using CpG-NFκB decoy strategy for targeting of the undruggable NF-κB transcription factor, a key tumorigenic regulator in B-cell lymphomas and many other human cancers.

### Example 2

Despite recent advances in the treatment of non-Hodgkin B-cell lymphoma (BCL), a significant number of patients relapse or remain refractory to current therapies. Treatment resistance in BCL is associated with survival signaling via NF-κB transcription factor. While NF-κB is an attractive molecular target in BCL and other human cancers, as a transcription factor it remains undruggable. Here, we describe a B-cell-selective NF-κB inhibitor consisting of a NF-κB-specific decoy oligodeoxynucleotide (dODN) conjugated to a CpG-B DNA sequence targeting Toll-like receptor-9 (TLR9)-expressing lymphoma cells. Bc-NFκBdODN showed efficient uptake by human diffuse large B-cell lymphoma (DLBCL) and mantle cell lymphoma (MCL) cells, such as U2932, OCI-Ly3, RL and Jekol, respectively. In addition, we confirmed that Bc-NFκBdODN inhibited nuclear translocation and DNA-binding activity of NF-κB together with the expression of CCND2 and MYC target genes. In vitro Bc-NFκBdODN enhanced direct and ionizing radiation-induced cytotoxicity in lymphoma cells. In xenotransplanted models of human U2932 and RL lymphomas, local injections of Bc-NFκBdODN reduced NF-κB activity in whole tumors and effectively arrested lymphoma progression when combined with a single, local 3 Gy dose of radiation. In immune-competent mice, intratumoral treatment with Bc-NFκBdODN suppressed growth of local and distant A20 tumors with evidence of immune-mediated anti-tumor responses. Our results underscore clinical relevance of this strategy, which can restore efficacy of standard radiation therapy to benefit patients with resistant or relapsed BCL.

B-cell lymphoma (BCL) is the most common type of non-Hodgkin lymphoma (NHL), accounting for roughly 85% of NHL cases in the United States.(1) Despite recent advances in BCL therapies, 30-40% patients will relapse or develop resistance to treatment. (2) Among different types of BCL, diffuse large B-cell lymphoma (DLBCL) is the most aggressive type of NHL with 40% percent of patients dying from the disease within five years from diagnosis. (3) Activated B-cell (ABC) subtype of DLBCL has a signature of aberrant NF-κB signaling resulting from oncogenic mutations in upstream signaling pathways such as B-cell receptor (BCR) or Toll-like receptor-9 (TLR9)/MyD88. (5,6) About 30% ABC DLBCL biopsies harbor MyD88 mutations that constitutively upregulates NF-κB gene expression signature. (6) Chronic active BCR signaling is also found in 15% of ABC-DLBCL and 7% of MCL involve mutations in Bruton tyrosine kinase (BTK), CARD11, among of others. (5,7,8) Knockdown of NF-κB upstream factors or the introduction of dominant negative regulators effectively reduce DLBCL cell viability and proliferation. (9) Similar effect can also be achieved by simultaneous knockdown of MyD88 and CARD11 in patient-derived ABC-DLBCL.(6) Furthermore, in addition to ABC DLBCL, NF-κB signaling is also responsible for oncogenesis and the progression of another aggressive subtype of B-cell NHL, mantle cell lymphoma (MCL), especially the relapsed or refractory MCL resistant to BTK inhibitors. (10)

The NF-κB family transcription factors comprise of NF-κB1 (p50 and its precursor p105), NF-κB2 (p52 and its precursor p100), RelA (p65), RelB and c-Rel, which forms homo- or heterodimers and initiates DNA transcription of target genes by binding to the specific κB enhancer element in the genome.(11) Canonical NF-κB signaling, involving p50, p65/RelA and c-Rel, play a critical role in pro-inflammatory cytokine production and immune cell functions.(12) Canonical NF-κB pathway is mediated by phosphorylation and degradation of NF-κB inhibitor IκBα through IκB kinase (IKK) complex containing IKKα, IKKβ and IKKγ (NEMO). Once activated, NF-κB dimers translocate into nucleus and regulate pro-inflammatory cytokine expression e.g. TNFα, (Interleukin) IL-6 and IL-12p40. (13) NF-κB also inhibits apoptosis by inducing c-Flip and Bcl-xL and promotes cell proliferation by enhancing cyclin D1 expression in normal and malignant cells.(14-16)

NF-κB signaling is an attractive target in cancer and inflammatory diseases given its central role in the regulation of cell survival and in the production of pro-inflammatory cytokines. (12,17,18) In addition, studies from our group and others have suggested a role for NF-κB in promoting therapeutic resistance of B-cell lymphoma and many solid tumors, including head and neck squamous cell carcinoma (HNSCC), breast cancer and glioma through upregulation of anti-apoptotic signaling. (1-4) Moreover, we previously reported that TLR9/NF-kB signaling in tumor-associated myeloid cells can shift the outcome of radiation therapy from immune activation to tumor revascularization and recurrence. (22) These effects on NF-κB activation resulted in production of IL-6 that triggers STAT3 mediated immunosuppression and angiogenesis. (22)

However, despite the high therapeutic potential of targeting NF-κB, there are currently no clinically approved, direct pharmacological inhibitors of NF-κB. (24) The majority of available small molecule NF-κB inhibitors including IKKβ or BTK inhibitor Ibrutinib target upstream kinases regulating NF-κB activity.(24,25) However, IKK inhibitors have not succeeded in clinical trials due to their limited therapeutic effects and off-target toxicities. (25,26) Alternatively, inhibitors of protein degradation can effectively interfere with IκB-mediated NF-κB activation. However, despite of outstanding effect in multiple myeloma and mantle cell lymphoma (MCL), proteasome inhibitors such as bortezomib did not demonstrate clinical benefit in hematological malignancies e.g. acute myeloid leukemia (AML), myelodysplastic syndrome (MDS) and acute lymphoblastic leukemia (ALL) and had significant off-target effects in non-malignant cells like plasma cells.(27) Nonetheless, while bortezomib had limited effect on DLBCL patients when applied alone, it significantly improves overall survival when combined with doxorubicin-based chemotherapy.(28) Thus, there is a high unmet need for an NF-κB inhibitor and novel combinational strategy efficacious in the context of refractory or relapsed BCL patients.

The challenges in the generation of small molecule inhibitors to NF-κB signaling underscore the need for alternative strategies. Since NF-κB interacts with specific DNA sequence, oligonucleotide therapeutics, such as decoy DNA could be an alternative approach to small molecules. The decoy ODNs comprise of a consensus DNA sequences recognized by NF-κB, thereby preventing its binding to regulatory elements in target gene promoters. NF-κB dODNs showed promising therapeutic activity in experimental models of inflammatory diseases, such as arthritis, using local delivery,(29,30) in asthma/pulmonary allergy after intranasal administration, (31) and in myocardial infarction using decoy injection into coronary artery. (32,33) NF-κB dODN also inhibited lung metastases in osteosarcoma models in mice. (34) However, clinical translation of decoy ODNs proved challenging, mainly due to the lack of targeted delivery methods. As a result, NF-κB dODNs have not achieved satisfactory results in initial clinical trials for atopic dermatitis and rheumatoid arthritis. (35,36) We have previously developed an approach for the targeted delivery of oligonucleotide therapeutics, such as siRNA, ASO or decoy molecules to TLR9-positive myeloid cells or B-cells in vivo.(37-39) Here, we adapted this approach to block survival NF-κB signaling in B-cell lymphoma cells in order to augment the efficacy of radiation therapy.

Materials and Methods

Cells

Human ABC-DLBCL cells OCI-Ly3, -Ly10 or U2932 cells were acquired. Human BCL cells (HBL-1) and MCL cells (JeKo-1, RL, REC-1) were obtained. Mouse BCL A20 cells were acquired from American Type Culture Collection (ATCC). RAWBlue cells were purchased from InvivoGen (San Diego, Calif.). Cells were cultured in RPMI 1640 with 20% (OCI-Ly3, -Ly10) or 10% (all others) fetal bovine serum (FBS). Cells were tested and confirmed as mycoplasma free using LookOut Mycoplasma PCR Detection kit (Sigma-Aldrich, St. Louis, Mo.).

Animal Studies

All animal experiments followed established institutional guidance and approved protocols from the Institutional Animal Care and Use Committee (CoH). BALB/c mice were purchased from the NCI/Charles River (Wilmington, Mass.). NOD/SCID/IL-2RgKO (NSG) mice, originally from the Jackson Laboratory (Bar Harbor, Me.) were maintained at CoH's animal facility. Mice were injected subcutaneously using 10⁷ of tested lymphoma cells in PBS and tumor progression was monitored using caliper measurements of tumor volume. Mice were treated using a single dose of the local tumor irradiation (15 Gy) under xylazine/ketamine anesthesia.

Oligonucleotide Design

All oligonucleotides were synthesized in the DNA/RNA Synthesis Core (CoH) by linking type-B CpG to the modified sequence of NF-κB decoy ODN (NFkBdODN) similarly as described previously. (43) The resulting conjugates are shown below (o=single C₃ unit, which has the following chemical structure:

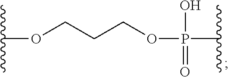 [SMILES: *OCCCOP(*)(=O)O]

asterisks indicate phosphorothioation sites):

Bc-NFkBdODN:
(SEQ ID NO: 13)
5′-T*C*C*A*T*G*A*C*G*T*T*C*C*T*G*A*T*G*C*T-ooooo-
C*C*T*TGAAGGGATTTCCCT CC-oooo-GGAGGGAAATCCCTTCA*A*
G*G*-ooo-(CH₂)₆NH₂-3′. SEQ ID NO: 13 has a 5′
terminal —OH.
Bc-scrODN:
(SEQ ID NO: 21)
5′-T*C*C*A*T*G*A*C*G*T*T*C*C*T*G*A*T*G*C*T-ooooo-
A*C*T*CTTGCCAATTAC-oooo-GTAATTGGCAAGA*G*T*-ooo-
(CH₂)₆NH₂-3′. SEQ ID NO: 21 has a 5′ terminal —OH.
NF-kBdODN:
(SEQ ID NO: 12)
5′-ooooo-C*C*T*TGAAGGGATTTCCCT CC-oooo-
GGAGGGAAATCCCTTCA *A*G*G-ooo-(CH₂)₆NH₂-3′

For internalization studies, oligonucleotides were 3′-labeled using Cy3 fluorochrome.

Electrophoretic Mobility Shift Assay (EMSA): EMSAs to detect DNA-binding NF-kB activity were performed as described previously. (39) Briefly, 10 pg nuclear extracts were incubated with 32P-labeled oligonucleotide probes specific to NF-kB (44). Anti-NF-κB p65, p50 or c-Rel antibodies were used for supershift controls to identify the proteins detected (Santa Cruz Biotechnology, Dallas, Tex.).

Confocal Microscopy: Imaging was performed using LSM 880 with Airyscan confocal microscope (Zeiss, Oberkochen, Germany). For uptake assays, cells were treated with Cy3-labeled CpG-NFkBdODN or NF-κB dODN then fixed on microscopy slides (Fisher Scientific, NH) with 2% PFA. For NF-κB relocation assays, RAW264.7 cells expressing p65-GFP fusion protein were seeded on 12-well plates and treated with 100 ng/mL LPS for 30 min before imaging.

Cell Apoptosis Assays: Cell death was detected using Annexin-V and 7AAD staining as described before. (45,46) Caspase-3 activity in apoptotic cells was examined using Caspase-Glo3/7 Assay (Promega, WI) and the reads were normalized by cell numbers detected using Cell-Titer Glo Assay (Promega) as described before. (47)

Statistical Analysis: An unpaired t test was used to calculate the two-tailed p value to estimate statistical significance of differences between two experimental groups. A one-way ANOVA plus Bonferroni posttest were applied to assess the statistical significance of differences between multiple treatment groups. The relationship between two groups was assessed using correlation and linear regression. The p values are indicated in the figures with asterisks: *p<0.05; **p<0.01; ***p<0.001. Data were analyzed using Prism v.6.03 software (GraphPad).

Cell-Selective Delivery of NF-κB Decoy to B-cell Lymphoma: The CpG-decoy conjugates allow for targeting “undruggable” transcription factors in TLR9-positive human and mouse immune cells as well as cancer cells, including B-cell lymphoma. (45) To adapt this strategy for targeting NF-κB, we selected several previously described decoy sequences and converted them into a nuclease-resistant hairpin design. (FIGS. 9A-9B). (44) The sequence with maximal NF-κB inhibitory effect in target mouse macrophages on the transcriptional activity of NF-κB was then conjugated via a synthetic linker to the 3′ end of a single-stranded B-class CpG ODN as a B cell-targeting domain (FIG. 1A). CpG and NF-κB decoy ODNs were either completely or partially phosphorothioated (PS), respectively, to enhance serum stability. Next, we compared the internalization of B cell-specific NFκBdODN (Bc-NFκBdODN) conjugate and the unconjugated NFκBdODN by target B-cell lymphoma cells. Consistently with previous studies, the mouse A20 BCL cells rapidly internalized the fluorescently-labeled Bc-NFκBdODN^{Cy3}, but not the NFκBdODN^{Cy3} alone, as detected by flow cytometry (FIG. 1D). The confocal microscopy confirmed intracellular/cytoplasmic localization of the conjugate in A20 cells (FIG. 1C). Furthermore, Bc-NFκBdODN showed robust uptake by a panel of human DLBCL cell lines, such as OCI-Ly3, OCI-Ly10, HBL-1, U2932, as well as JeKo-1, RL and REC-1 mantle cell lymphoma cells even at 50 nM low concentration (FIG. 1E). Bc-NFκBdODN was selectively internalized by primary mouse immune cells such as DCs, granulocytic cells, macrophages and B cells, but not by T cells even after overnight incubation (FIG. 1F). Overall, our results demonstrated a pattern of cell selective Bc-NFκBdODN uptake consistent with the scavenger receptor-/TLR9-dependent mechanism of internalization of other CpG-conjugates by myeloid and B-cell lineage cells. (45)

Targeted inhibition of NF-κB activity in BCL cells and immune cells: To characterize the effect of Bc-NFκBdODN in target cells, we first used confocal microscopy to assess the cellular localization of NF-κB in mouse RAW264.7 cells expressing NF-κB RelA/p65-GFP fusion protein. The cells were incubated with Bc-NFκBdODN or control Bc-scrODN and stimulated with LPS. The imaging revealed that Bc-NFκBdODN, but not by Bc-scrODN, prevented LPS-induced nuclear translocation of p65-GFP (FIG. 2B). The NF-κB DNA-binding activity was further assessed using electrophoretic mobility shift assay (EMSA) in the primary mouse immune cells. As shown in FIG. 2A, Bc-NFκBdODN significantly reduced DNA-binding of NF-κB complexes induced by LPS, which comprised mainly of p65/p50 and heterodimers, but not c-Rel, as verified using antibodies specific to each NF-κB subunit. Similar results were derived from human and mouse ABC-DLBCL and MCL cells treated using Bc-NFκBdODN (FIG. 2A). Bc-NFκBdODN eliminated almost completely constitutive nuclear activity of NF-κB in mouse BCL A20 cells, human ABC-DLBCL U2932 cells and human mantle cell lymphoma RL cells. Control unconjugated NF-κB dODN had minimal inhibitory effect, while Bc-scrODN had stimulatory activity in DLBCL cells but not in RL cells, likely resulting from TLR9 activation. Finally, only Bc-NFκBdODN but not the unconjugated decoy or Bc-scrODN, significantly inhibited the NF-κB downstream gene expressions including CCND2 and MYC in U2932 cells as measured using qPCR (FIG. 2C). Together, these results provide evidence that Bc-NFκBdODN is an effective NF-κB inhibition in BCL cells and primary myeloid cells.

Bc-NFκBdODN limits survival signaling in human BCL cells in vitro: NF-κB signaling promotes survival of B-cell lymphoma cells and is associated with lymphoma resistance to therapies. (5,6,19) Thus, we tested direct cytotoxic effects of Bc-NFκBdODN on cultured human ABC-DLBCL cells. OCI-Ly3 and U2932 cells were incubated in the presence of Bc-NFκBdODN or control Bc-scrODN before the assessment of cell viability using flow cytometry after Annexin-V and 7AAD staining. The Bc-NFκBdODN treatment induced apoptosis in ˜60% of Ly3 and ˜40% of U2932 cells on average (FIG. 2F). Correspondingly, both Ly3 and U2932 cells showed dose-dependent elevation of caspase-3 activity starting from 0.5-1 pM concentration. (FIG. 2D) Control Bc-scrODN had some effect on the viability of Ly3 cells, but not on U2932 cells up to 1 pM concentration (FIG. 2D). Next, we assessed whether the NF-κB blockade would improve therapeutic effects of radiation therapy or proteasome inhibitor, bortezomib. As shown in FIG. 2E, Bc-NFκBdODN, but not the control Bc-scrODN, significantly enhanced the apoptotic effect of irradiation in both Ly3 and U2932 cells even at low 0.1 pM concentration of the conjugate. Bc-NFκBdODN also enhanced the cytotoxic effect of bortezomib, a clinically relevant small molecule proteasome inhibitor on U2932 cells (FIG. 2E, right panel). These results underscore the potential of using decoy approach to boost efficacy of clinical B-cell lymphoma therapies.

In vivo targeting of human lymphoma xenotransplants using Bc-NFkBdODN: The in vitro activity of Bc-NFκBdODN prompted us to test the therapeutic effects in mouse BCL models. To verify the inhibitory effect of the conjugate, established xenotransplanted human U2932 lymphoma tumors were treated intratumorally using 10 mg/kg Bc-NFκBdODN, Bc-scrODN or PBS daily for three days. The inhibition of NF-κB activity was verified using immunohistochemical staining to assess nuclear localization of NF-κB. As shown in FIG. 3C, Bc-NFκBdODN treatment reduced nuclear localization of NF-κB compared to control Bc-scrODN or PBS treated tumors similar as observed in vitro previously (FIG. 2B). These results were further confirmed using EMSA assay, which detected reduced NF-κB DNA-binding activity (FIG. 3E). Correspondingly, immunohistochemical staining for caspase-3 activity revealed significantly higher apoptosis in Bc-NFκBdODN-treated tumors vs. control Bc-scrODN or PBS tumors (FIG. 3E). Finally, Bc-NFκBdODN significantly reduced expression of CCND2, a downstream NF-κB target gene, in U2932 tumors (FIG. 3D). Next, we assessed antitumor efficacy of Bc-NFκBdODN used as a single agent or in combination with local tumor irradiation in human ABC-DLBCL xenotransplants. NSG mice with established subcutaneous Ly3 lymphomas were treated using 10 mg/kg Bc-NFκBdODN, Bc-scrODN or PBS injected intratumorally daily for 21 days. The mice were monitored for tumor regrowth after the end of treatment. Interestingly, although both Bc-NFκBdODN and Bc-scrODN induces expressive decrease in the tumor volume at the beginning, likely due to potent effect of TLR9 mediated immunostimulation at the selected concentration as reported before (48). Tumor regression was retained longer in the Bc-NFκBdODN treated group, while Bc-scrODN treated tumors relapsed rapidly after treatment stopped (FIG. 3A). Similar effect was also observed in Ly3 tumor models, in which 10 mg/kg Bc-NFκBdODN induced tumor regression while Bc-scrODN only delayed tumor growth but failed to shrink the tumor (FIG. 3B). All together, we confirmed the therapeutic effect of Bc-NFκBdODN on human DLBCL tumors.

NF-κB inhibition sensitizes xenograft human ABC-DLBCL to radiation therapy: Since the combination of the ionizing irradiation and Bc-NFκBdODN demonstrated synergistic effect in vitro, we assessed whether the Bc-NFκBdODN will sensitize DLBCL tumors to local radiation in vivo. The established, subcutaneously growing Ly3 tumors were treated intratumorally twice with 5 mg/kg Bc-NFκBdODN or scrambled control before 3 Gy irradiation. After irradiation, the mice were then kept on daily Bc-NFκBdODN or Bc-scrODN treatment. Of note, combination of irradiation and Bc-NFκBdODN showed superior synergistic anti-tumor effect which rapidly induced tumor regression and prevented tumor relapse till the end of the experiment. On the contrary, irradiation alone only induced tumor regression at the beginning but failed to control tumor regrowth later, and combination with Bc-scrODN did not improve the effect by irradiation alone. 5 mg/kg Bc-NFκBdODN treatment showed similar tumor growth pattern as irradiation alone, while Bc-scrODN only have marginal effect in disease progression (FIG. 7). Similar effect was also observed in human mantle cell lymphoma RL cell xenograft model receiving the combinational therapy (FIG. 4B). The inhibition of NF-κB activity by Bc-NFκBdODN in combination with irradiation was also confirmed by reduction of NF-κB downstream genes CCND2 and TNF expression (FIG. 4C). These data supported the rationale of irradiation plus selective NF-κB activity for the treatment of lymphoma and suggested potential clinical application of the combinational therapy.

Disruption of the NF-kB signaling triggers immune-mediated anti-tumor responses in syngenic model of mouse B-cell lymphoma: NF-κB operates in both lymphoma cells as well as in non-malignant immune cells, such as DCs, macrophages and B cells often supporting their functions. Thus, we have assessed whether NF-κB inhibition in both compartment will support or diminish the antitumor efficacy of Bc-NFκBdODN in immunocompetent mice. We used A20 lymphoma cells with tumors injected s.c. on both sides of the abdomen. Such dual tumor model allowed for the assessment of oligonucleotide effects both locally, at the treated site, and systemically, at the distant/untreated tumor site. We first verified that Cy3-labeled oligonucleotides injected in the primary tumor do not reach distant tumors (FIG. 10). As shown in FIG. 8A left panel, both Bc-NFκBdODN and Bc-scrODN induced complete tumor regression at the treated site, likely due to potent effect of TLR9 mediated immunostimulation at the selected concentration as reported before. (48) However, only Bc-NFκBdODN suppressed tumor growth in the opposite site suggesting potential generation of systemic antitumor immune responses (FIG. 8A, right panel). Importantly, the flow cytometric analysis of distant tumors revealed that Bc-NFκBdODN significantly enhanced T cell infiltration, especially the CD4⁺ T cells but not CD8⁺ T cells, compared to control treatments (FIG. 8B). Both CD4⁺ (FIG. 8C) and CD8⁺ T cell (FIG. 8D) infiltrating tumors showed lower PD-1 expression. In parallel, we also observed reduced PD-L1 expression on CD11b⁺ cells in tumors (FIG. 8E). In conclusion, the disruption of NF-κB signaling in the lymphoma microenvironment led to systemic activation of T-cell antitumor immune responses.

In this study, we demonstrate the potential of using decoy-based Bc-NFκBdODN strategy for targeting NF-κB signaling specifically in B cell and mantle cell lymphomas, thereby reducing cancer cell survival and augmenting lymphoma sensitivity to radiation therapy. When conjugated together, the Bc-NFκBdODN prevented the nucleus translocation of activated NF-κB dimers and blocked NF-κB activity in the nucleus. It also significantly inhibited NF-κB downstream gene expression and induced apoptosis in BCL cells in vitro and in vivo. Importantly, Bc-NFκBdODN showed efficacy in vivo inhibiting NF-κB signaling and inducing tumor cell death and leading to regression of both human and mouse lymphoma models in mice. The effect of Bc-NFκBdODN was synergistic with cancer cell irradiation. Local administration of Bc-NFκBdODN induced lymphoma regression when combined with a single dose of radiation therapy. Since radiotherapy is a standard regimen widely used in BCL patients, these results indicate clinical relevance of our strategy Bc-NFκB. Interestingly, we found that Bc-NFκBdODN treatment suppressed distant tumor growth in immune competent mice and converted immune-suppressive TME in the opposite site, which indicates selective suppression of NF-κB in tumor cells and tumor-associated myeloid cells induced immune-mediated anti-tumor effects. The mechanism of these abscopal effects seems to rely on the immune activation, reduction of PD1/PD-L1 immune checkpoint regulation and improved T-cell recruitment into tumors and will be further explored in future studies. The promising recent results demonstrating synergism of CTLA4 blockade with radiation therapy in NSCLC patients suggest that other immune checkpoints inhibitors can be tested in combination with C-NFkBdODN and tumor irradiation. (49)

Multiple approaches have been utilized to improve the effect of existing chemo- or radiation therapies by combining NF-κB inhibition. For example, bortezomib sensitizes recurrent DLBCL to doxorubicin-based chemotherapy. (28) Detailed analysis found that inhibition of NF-κB prevented DNA damage repair in response to doxorubicin treatment and lead to chromosome instability in DLBCL patients.(50) Besides that, blockage of NF-κB activation sensitizes prostate cancer cells to paclitaxel or ionizing radiation-induced apoptosis (51,52). These studies suggested the feasibility of combining NF-κB inhibition with genotoxic or radiation therapies. In addition to survival and resistance to chemo- and radiation therapies, NF-κB has been reported to facilitate immune suppression by tumor cells. It plays important roles in PD-L1 activation through strong binding on PD-L1 and enhancer 9 promoter. (53,54) Besides lymphoma cells, NF-kB activity in tumor associated myeloid cells also promotes immune suppression and tumor angiogenesis by driving expression of cytokines like IL-6. (22,55,56) We have shown that IL-6 induced by TLR9/NF-κB axis after radiation enhances STAT3 activity in tumor-associated myeloid cells and promotes immune suppression and revascularization that favors oncogenesis.(22) Based on these findings, NF-κB inhibition could break the immune suppression by both tumor cells and tumor-associated myeloid cells through multiple mechanisms. In addition to that, NF-κB subunits confer different functions in survival and immune response. NF-κB1/p50 and RelA/p65 is one of the most stable heterodimer among all NF-κB subsets.(11) RelA promotes mouse embryonic fibroblasts survival in response to TNF-associated apoptosis while c-Rel acts in the opposite way.(57) RelA is also more important for type I IFN expression in dendritic cells (DCs), while c-Rel is required for DC activation and survival after CpG stimulation.(58,59) c-Rel is also critical for the activation and differentiation of mature B cells. (60) In this study, Bc-NFκBdODN specifically interact with NF-κB1/p50 and RelA/p65 but not c-Rel, which precisely inhibits tumor-promoting inflammation while preserves necessary immune cell functions.

On the other hand, global inhibition of NF-κB without cell selectivity may increase the risk of side effects. Since NF-κB is critical for T cell activation, broad abrogation of NF-κB activities in immune cells could compromise host defense to infection (12,61). Mouse studies also showed that global ikkb^{-/-} and rela^{-/-} leads to embryonic lethal liver apoptosis. (62,63) Therefore, selective inhibition of NF-κB signaling in tumor and the TME-associated myeloid cells is essential for successful anti-tumor immune responses and to avoid toxicity due to broad NF-κB inhibition. This can be achieved with our CpG ODN-conjugation strategy, which facilitates rapid cellular uptake and cell-specific delivery to myeloid cells in vivo, (40-42) while leaving T cell activation intact.

TLR9 stimulation reagent CpG ODN inhibits proliferation of B cell lymphoma cells in vitro. In murine BCL, CpG ODN induced apoptosis by decreasing Bcl-xL and enhancing Fas/Fas-L expression(64) and in human B-cell chronic lymphocytic leukemia (B-CLL), CpG ODN treatments leads to JAK/STAT1-mediated Caspase3 cleavage and apoptosis.(65) However, the outcome of CpG treatment in vivo is controversial. For example, supernatant from in vivo implanted murine BCL tumors can suppress the inhibitory effect of CpG, indicating interference by tumor microenvironment.(66) Many approaches have been tested to improve CpG ODN's effect in lymphoma by combination with radiation, BTK inhibitor ibrutinib, and monoclonal antibodies including rituximab, anti-OX-40 and anti-CTLA-4 antibody among others to improve their therapeutic effects.(67-71) Interestingly, in another study using subcutaneous mouse BCL A20 model, combined treatment using CpG ODN with ibrutinib induced tumor regression in both treated and opposite site, which is similar to our findings. The study suggested that ibrutinib can trigger immunogenic cell-death with tumor antigen release, while CpG stimulation enhances the tumor antigen presentation by APCs to induce CD4⁺ and CD8⁺ T cells-mediated anti-tumor immune responses.(67) These results were also supported by the recent clinical study in low-grade BCL patients treated using CpG ODN and ibrutinib, in which increased CD4⁺ T cell infiltration was observed in both injected site and distant sites.(68) Overall, these observations highlight the feasibility of inhibiting NF-κB signaling while promoting anti-tumor immune response through TLR9 stimulation in B-cell malignancies. Correspondingly, our data postulates that a combination of CpG ODN and NF-κB inhibitor in one molecule can optimize the outcome of TLR9 signaling in B-cell lymphoma. The Bc-NFκBdODN conjugate minimizes release of potentially tumor-promoting cytokines, such as IL-6 or IL-10, while preserving the inhibitory effect in downstream NF-κB targets among survival genes such as MYC or CCND2. As a result, the Bc-NFκBdODN oligonucleotides can effectively sensitizes B-cell lymphoma to radiation therapy. More importantly, the Bc-NFκBdODN strategy overcomes the immunosuppressive tumor microenvironment and thereby facilitates the systemic antitumor immune responses. Further studies will investigate more detailed molecular and cellular mechanisms of these immune mediated effects to identify additional therapeutic targets as well as predictive and/or prognostic biomarkers.

TABLE 1

| | | |
|---|---|---|
| NF-kB decoy 1A (13 nt) | No | 5′ ppppp-TGG GGA CTT TCC A-pppp-T GGA AAG |
| SEQ ID NO: 1 | | TCC CCA-pppp 3′ |
| NF-kB decoy 1B (13 nt) | No | 5′ ppppp-T GGA AAG TCC CCA-pppp-TGG GGA |
| SEQ ID NO: 2 | | CTT TCC A-pppp 3′ |
| NF-kB decoy 2 (22 nt) | *PS | 5′ ppppp-G*A*T* CGA GGG GAC TTT CCC TAG C- |
| SEQ ID NO: 3 | | pppp-G CTA GGG AAA GTC CCC TCG *A*T*C- |
| | | pppp 3′ |
| NF-kB decoy 3 (20 nt) | *PS | 5′ ppppp-C*C*T* TGA AGG GAT TTC CCT CC- |
| SEQ ID NO: 4 | | pppp-GG AGG GAA ATC CCT TCA *A*G*G-pppp |
| | | 3′ |
| CpG(1668)-NF-kB decoy | *PS | 5′ |
| 3 | | T*C*C*A*T*G*A*C*G*T*T*C*C*T*G*A*T*G*C*T- |
| SEQ ID NO: 5 | | ppppp-C*C*T* TGA AGG GAT TTC CCT CC- |
| | | pppp-GG AGG GAA ATC CCT TCA *A*G*G-pppp |
| | | 3′ |
| GpC (1668)-NF-kB | *PS | 5′ |
| decoy 3 | | T*C*C*A*T*G*A*G*C*T*T*C*C*T*G*A*T*G*C*T- |
| SEQ ID NO: 6 | | ppppp-C*C*T* TGA AGG GAT TTC CCT CC- |
| | | pppp-GG AGG GAA ATC CCT TCA *A*G*G-pppp |
| | | 3′ |
| CpG(D19)-NF-kB decoy | *PS | 5′ G*G*T GCA TCG ATG CAG G*G*G*G*G-ppppp- |
| 3 | | C*C*T* TGA AGG GAT TTC CCT CC-pppp-GG |
| SEQ ID NO: 7 | | AGG GAA ATC CCT TCA *A*G*G-pppp 3′ |
| GpC(D19)-NF-kB decoy | *PS | 5′ G*G*T GCA TGC ATG CAG G*G*G*G*G-ppppp- |
| 3 | | C*C*T* TGA AGG GAT TTC CCT CC-pppp-GG |
| SEQ ID NO: 8 | | AGG GAA ATC CCT TCA *A*G*G-pppp 3′ |
| #1 | | 5′-ooooo-TGG GGA CTT TCC A-oooo-T GGA |
| SEQ ID NO: 1 | | AAG TCC CCA-oooo-3′ |
| #2 | | 5′-ooooo T GGA AAG TCC CCA-oooo TGG |
| SEQ ID NO: 2 | | GGA CTT TCC A-oooo-3′ |
| #3 | | 5′-ooooo-G*A*T* CGA GGG GAC TTT CCC TAG |
| SEQ ID NO: 3 | | C-oooo-GCTAGGGAAAGTCCCC TCG*A*T*C- |
| | | oooo-3′ |
| #4 | | 5′-ooooo-C*C*T*TGA AGG GAT TTC CCT CC- |
| SEQ ID NO: 4 | | 0000-GG AGG GAA ATC CCT TCA* A*G*G-oooo- |
| | | 3′ |
| NF-kB decoy 1A (13 nt) | No | 5′ ppppp-TGG GGA CTT TCC A-pppp-T GGA AAG |
| SEQ ID NO: 9 | | TCC CCA-ppp-(CH₂)₆NH₂ 3′ |
| NF-kB decoy 1B (13 nt) | No | 5′ ppppp-T GGA AAG TCC CCA-pppp-TGG GGA |
| SEQ ID NO: 10 | | CTT TCC A-ppp-(CH₂)₆NH₂ 3′ |
| NF-kB decoy 2 (22 nt) | *PS | 5′ ppppp-G*A*T* CGA GGG GAC TTT CCC TAG C- |
| SEQ ID NO: 11 | | pppp-G CTA GGG AAA GTC CCC TCG *A*T*C- |
| | | ppp-(CH₂)₆NH₂ 3′ |
| NF-kB decoy 3 (20 nt) | *PS | 5′ ppppp-C*C*T* TGA AGG GAT TTC CCT CC- |
| SEQ ID NO: 12 | | pppp-GG AGG GAA ATC CCT TCA *A*G*G-ppp- |
| | | (CH₂)₆NH₂ 3′ |
| CpG(1668)-NF-kB decoy | *PS | 5′ |
| 3 | | T*C*C*A*T*G*A*C*G*T*T*C*C*T*G*A*T*G*C*T- |
| SEQ ID NO: 13 | | ppppp-C*C*T* TGA AGG GAT TTC CCT CC- |
| | | pppp-GG AGG GAA ATC CCT TCA *A*G*G-ppp- |
| | | (CH₂)₆NH₂ 3′ |
| GpC (1668)-NF-kB | *PS | 5′ |
| decoy 3 | | T*C*C*A*T*G*A*G*C*T*T*C*C*T*G*A*T*G*C*T- |
| SEQ ID NO: 14 | | ppppp-C*C*T* TGA AGG GAT TTC CCT CC- |
| | | pppp-GG AGG GAA ATC CCT TCA *A*G*G-ppp- |
| | | (CH₂)₆NH₂ 3′ |
| CpG(D19)-NF-kB decoy | *PS | 5′ G*G*T GCA TCG ATG CAG G*G*G*G*G-ppppp- |
| 3 | | C*C*T* TGA AGG GAT TTC CCT CC-pppp-GG |
| SEQ ID NO: 15 | | AGG GAA ATC CCT TCA *A*G*G-ppp-(CH₂)₆NH₂ |
| | | 3′ |
| GpC(D19)-NF-kB decoy | *PS | 5′ G*G*T GCA TGC ATG CAG G*G*G*G*G-ppppp- |
| 3 | | C*C*T* TGA AGG GAT TTC CCT CC-pppp-GG |
| SEQ ID NO: 16 | | AGG GAA ATC CCT TCA *A*G*G-ppp-(CH₂)₆NH₂ |
| | | 3′ |
| #1 | | 5′-ooooo-TGG GGA CTT TCC A-oooo-T GGA |
| SEQ ID NO: 9 | | AAG TCC CCA-ooo-(CH₂)₆NH₂-3′ |
| #2 | | 5′-ooooo-T GGA AAG TCC CCA-oooo-TGG |
| SEQ ID NO: 10 | | GGA CTT TCC A-ooo-(CH₂)₆NH₂-3′ |
| #3 | | 5′-ooooo-G*A*T* CGA GGG GAC TTT CCC TAG |
| SEQ ID NO: 11 | | C-oooo-GCTAGGGAAAGTCCCC TCG*A*T*C- |
| | | ooo-(CH₂)₆NH₂-3′ |
| #4 | | 5′-ooooo-C*C*T*TGA AGG GAT TTC CCT CC- |
| SEQ ID NO: 12 | | oooo-GG AGG GAA ATC CCT TCA* A*G*G-ooo- |
| | | (CH₂)₆NH₂-3′ |
| Bc-scrODN: | | 5′- |
| SEQ ID NO: 17 | | T*C*C*A*T*G*A*C*G*T*T*C*C*T*G*A*T*G*C*T- |
| | | ooooo-A*C*T*CTTGCCAATTAC-oooo- |
| | | GTAATTGGCAAGA*G*T*-oooo-3′ |
| Bc-scrODN: | | 5′- |
| SEQ ID NO: 21 | | T*C*C*A*T*G*A*C*G*T*T*C*C*T*G*A*T*G*C*T- |
| | | ooooo-A*C*T*CTTGCCAATTAC-oooo- |
| | | GTAATTGGCAAGA*G*T*-ooo-(CH₂)₆NH₂-3′ |

### SEQUENCE LISTING

The sequences described below have been marked with a ‘*’ which is indicative of a phosphorothioate linkage. The symbol “p” and “o” are an internal C3 spacer, which has the following chemical structure:

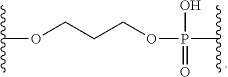 [SMILES: *OCCCOP(*)(=O)O]

SEQ ID NO: 5, SEQ ID NO: 6, SEQ ID NO: 7, SEQ ID NO: 8, SEQ ID NO: 13, SEQ ID NO: 14, SEQ ID NO: 15, SEQ ID NO: 16, SEQ ID NO: 17, and SEQ ID NO: 21 have a 5′ terminal —OH.

### REFERENCES

1. Siegel R L, Miller K D, Jemal A. Cancer statistics, 2016. C A Cancer J Clin. 2016 February; 66(1):7-30.
2. Klyuchnikov E, Bacher U, Kroll T, Shea T C, Lazarus H M, Bredeson C, et al. Allogeneic hematopoietic cell transplantation for diffuse large B cell lymphoma: who, when and how? Bone Marrow Transplant. 2014 January; 49(1):1-7.
3. Diffuse Large B-Cell Lymphoma—Cancer Stat Facts [Internet]. [cited 2019 May 10]. Available from: https://seer.cancer.gov/statfacts/html/dlbcl.html
4. Fu S, Wang M, Lairson D R, Li R, Zhao B, Du X L. Trends and variations in mantle cell lymphoma incidence from 1995 to 2013: A comparative study between Texas and National SEER areas. Oncotarget. 2017 Dec. 22; 8(68):112516-112529.
5. Davis R E, Ngo V N, Lenz G, Tolar P, Young R M, Romesser P B, et al. Chronic active B-cell-receptor signalling in diffuse large B-cell lymphoma. Nature. 2010 Jan. 7; 463(7277):88-92.
6. Ngo V N, Young R M, Schmitz R, Jhavar S, Xiao W, Lim K-H, et al. Oncogenically active MYD88 mutations in human lymphoma. Nature. 2011 Feb. 3; 470(7332):115-119.
7. Lenz G, Davis R E, Ngo V N, Lam L, George T C, Wright G W, et al. Oncogenic CARD11 mutations in human diffuse large B cell lymphoma. Science. 2008 Mar. 21; 319(5870):1676-1679.
8. Young R M, Staudt L M. Targeting pathological B cell receptor signalling in lymphoid malignancies. Nat Rev Drug Discov. 2013 March; 12(3):229-243.
9. Davis R E, Brown K D, Siebenlist U, Staudt L M. Constitutive nuclear factor kappaB activity is required for survival of activated B cell-like diffuse large B cell lymphoma cells. J Exp Med. 2001 Dec. 17; 194(12):1861-1874.
10. Balaji S, Ahmed M, Lorence E, Yan F, Nomie K, Wang M. NF-κB signaling and its relevance to the treatment of mantle cell lymphoma. J Hematol Oncol. 2018 Jun. 15; 11(1):83.
11. Huxford T, Ghosh G. A structural guide to proteins of the NF-kappaB signaling module. Cold Spring Harb Perspect Biol. 2009 September; 1(3):a000075.
12. Liu T, Zhang L, Joo D, Sun S-C. NF-κB signaling in inflammation. Signal transduction and targeted therapy. 2017 Jul. 14; 2.
13. Kawai T, Akira S. The role of pattern-recognition receptors in innate immunity: update on Toll-like receptors. Nat Immunol. 2010 May; 11(5):373-384.
14. Micheau O, Lens S, Gaide O, Alevizopoulos K, Tschopp J. NF-kappaB signals induce the expression of c-FLIP. Mol Cell Biol. 2001 August; 21(16):5299-5305.
15. Wurster A L, Rodgers V L, White M F, Rothstein T L, Grusby M J. Interleukin-4-mediated protection of primary B cells from apoptosis through Stat6-dependent up-regulation of Bcl-xL. J Biol Chem. 2002 Jul. 26; 277(30):27169-27175.
16. Joyce D, Albanese C, Steer J, Fu M, Bouzahzah B, Pestell R G. NF-kappaB and cell-cycle regulation: the cyclin connection. Cytokine Growth Factor Rev. 2001 March; 12(1):73-90.
17. Karin M. NF-kappaB as a critical link between inflammation and cancer. Cold Spring Harb Perspect Biol. 2009 November; 1(5):a000141.
18. Jost P J, Ruland J. Aberrant NF-kappaB signaling in lymphoma: mechanisms, consequences, and therapeutic implications. Blood. 2007 Apr. 1; 109(7):2700-2707.
19. Bernal-Mizrachi L, Lovly C M, Ratner L. The role of NF-{kappa}B-1 and N F-{kappa}B-2-mediated resistance to apoptosis in lymphomas. Proc Natl Acad Sci USA. 2006 Jun. 13; 103(24):9220-9225.
20. Ahmed K M, Cao N, Li J J. HER-2 and NF-kappaB as the targets for therapy-resistant breast cancer. Anticancer Res. 2006 December; 26(6B):4235-4243.
21. Bhat K P L, Balasubramaniyan V, Vaillant B, Ezhilarasan R, Hummelink K, Hollingsworth F, et al. Mesenchymal differentiation mediated by NF-κB promotes radiation resistance in glioblastoma. Cancer Cell. 2013 Sep. 9; 24(3):331-346.
22. Gao C, Kozlowska A, Nechaev S, Li H, Zhang Q, Hossain D M S, et al. TLR9 signaling in the tumor microenvironment initiates cancer recurrence after radiotherapy. Cancer Res. 2013 Dec. 15; 73(24):7211-7221.
23. Sampath S, Won H, Massarelli E, Li M, Frankel P, Vora N, et al. Combined modality radiation therapy promotes tolerogenic myeloid cell populations and STAT3-related gene expression in head and neck cancer patients. Oncotarget. 2018 Feb. 16; 9(13):11279-11290.
24. Bennett J, Capece D, Begalli F, Verzella D, D'Andrea D, Tornatore L, et al. NF-κB in the crosshairs: Rethinking an old riddle. Int J Biochem Cell Biol. 2018; 95:108-112.
25. Prescott J A, Cook S J. Targeting ikkβ in cancer: challenges and opportunities for the therapeutic utilisation of ikko inhibitors. Cells. 2018 Aug. 23; 7(9).
26. Yamamoto Y, Gaynor R B. Therapeutic potential of inhibition of the NF-kappaB pathway in the treatment of inflammation and cancer. J Clin Invest. 2001 January; 107(2):135-142.
27. Manasanch E E, Orlowski R Z. Proteasome inhibitors in cancer therapy. Nat Rev Clin Oncol. 2017 July; 14(7):417-433.
28. Dunleavy K, Pittaluga S, Czuczman M S, Dave S S, Wright G, Grant N, et al. Differential efficacy of bortezomib bortezomib plus chemotherapy within molecular subtypes of diffuse large B-cell lymphoma. Blood. 2009 Jun. 11; 113(24):6069-6076.
29. Tomita T, Takeuchi E, Tomita N, Morishita R, Kaneko M, Yamamoto K, et al. Suppressed severity of collagen-induced arthritis by in vivo transfection of nuclear factor kappaB decoy oligodeoxynucleotides as a gene therapy. Arthritis Rheum. 1999 December; 42(12):2532-2542.
30. Tomita T, Takano H, Tomita N, Morishita R, Kaneko M, Shi K, et al. Transcription factor decoy for NFkappaB inhibits cytokine and adhesion molecule expressions in synovial cells derived from rheumatoid arthritis. Rheumatology (Oxford). 2000 July; 39(7):749-757.
31. Desmet C, Gosset P, Pajak B, Cataldo D, Bentires-Alj M, Lekeux P, et al. Selective blockade of N F-kappa B activity in airway immune cells inhibits the effector phase of experimental asthma. J Immunol. 2004 Nov. 1; 173(9):5766-5775.
32. Yamasaki K, Asai T, Shimizu M, Aoki M, Hashiya N, Sakonjo H, et al. Inhibition of NFkappaB activation using cis-element “decoy” of NFkappaB binding site reduces neointimal formation in porcine balloon-injured coronary artery model. Gene Ther. 2003 February; 10(4):356-364.
33. Morishita R, Sugimoto T, Aoki M, Kida I, Tomita N, Moriguchi A, et al. In vivo transfection of cis element “decoy” against nuclear factor-κB binding site prevents myocardial infarction. Nat Med. 1997 August; 3(8):894-899.
34. Nishimura A, Akeda K, Matsubara T, Kusuzaki K, Matsumine A, Masuda K, et al. Transfection of NF-κB decoy oligodeoxynucleotide suppresses pulmonary metastasis by murine osteosarcoma. Cancer Gene Ther. 2011 April; 18(4):250-259.
35. NF-kappaB Decoy oligonucleotide—Anesiva—AdisInsight [Internet]. [cited 2019 May 10]. Available from: https://adisinsight.springer.com/drugs/800018437
36. Fabre S, Apparailly F. Gene therapy for rheumatoid arthritis: current status and future prospects. BioDrugs. 2011 Dec. 1; 25(6):381-391.
37. Moreira D, Adamus T, Zhao X, Su Y-L, Zhang Z, White S V, et al. STAT3 Inhibition Combined with CpG Immunostimulation Activates Antitumor Immunity to Eradicate Genetically Distinct Castration-Resistant Prostate Cancers. Clin Cancer Res. 2018 Dec. 1; 24(23):5948-5962.
38. Zhang Q, Hossain DMS, Duttagupta P, Moreira D, Zhao X, Won H, et al. Serum-resistant CpG-STAT3 decoy for targeting survival and immune checkpoint signaling in acute myeloid leukemia. Blood. 2016 Mar. 31; 127(13):1687-1700.
39. Zhao X, Zhang Z, Moreira D, Su Y-L, Won H, Adamus T, et al. B Cell Lymphoma Immunotherapy Using TLR9-Targeted Oligonucleotide STAT3 Inhibitors. Mol Ther. 2018 Mar. 7; 26(3):695-707.
40. Hossain DMS, Moreira D, Zhang Q, Nechaev S, Swiderski P, Kortylewski M. TLR9-Targeted SiRNA Delivery In Vivo. Methods Mol Biol. 2016; 1364:183-196.
41. Kortylewski M, Nechaev S. How to train your dragon: targeted delivery of microRNA to cancer cells in vivo. Mol Ther. 2014 June; 22(6):1070-1071.
42. Kortylewski M, Nechaev S. Cancer therapy using oligonucleotide-based STAT3 inhibitors: will they deliver? Ther Deliv. 2014 March; 5(3):239-242.
43. Kortylewski M, Swiderski P, Herrmann A, Wang L, Kowolik C, Kujawski M, et al. In vivo delivery of siRNA to immune cells by conjugation to a TLR9 agonist enhances antitumor immune responses. Nat Biotechnol. 2009 October; 27(10):925-932.
44. Wan F, Lenardo M J. Specification of DNA binding activity of NF-kappaB proteins. Cold Spring Harb Perspect Biol. 2009 October; 1(4):a000067.
45. Zhang Q, Hossain DMS, Nechaev S, Kozlowska A, Zhang W, Liu Y, et al. TLR9-mediated siRNA delivery for targeting of normal and malignant human hematopoietic cells in vivo. Blood. 2013 Feb. 21; 121(8):1304-1315.
46. Qaqish A, Huang D, Chen C Y, Zhang Z, Wang R, Li S, et al. Adoptive Transfer of Phosphoantigen-Specific γδ T Cell Subset Attenuates Mycobacterium tuberculosis Infection in Nonhuman Primates. J Immunol. 2017 Jun. 15; 198(12):4753-4763.
47. Zhang Z, Yang E, Hu C, Cheng H, Chen C Y, Huang D, et al. Cell-Based High-Throughput Screening Assay Identifies 2′,2′-Difluoro-2′-deoxycytidine Gemcitabine as a Potential Antipoliovirus Agent. ACS infectious diseases. 2017 Jan. 13; 3(1):45-53.
48. Krieg A M. CpG still rocks! Update on an accidental drug. Nucleic Acid Ther. 2012 April; 22(2):77-89.
49. Formenti S C, Rudqvist N-P, Golden E, Cooper B, Wennerberg E, Lhuillier C, et al. Radiotherapy induces responses of lung cancer to CTLA-4 blockade. Nat Med. 2018 Nov. 5; 24(12):1845-1851.
50. Ramachandiran S, Adon A, Guo X, Wang Y, Wang H, Chen Z, et al. Chromosome instability in diffuse large B cell lymphomas is suppressed by activation of the noncanonical NF-κB pathway. Int J Cancer. 2015 May 15; 136(10):2341-2351.
51. Flynn V, Ramanitharan A, Moparty K, Davis R, Sikka S, Agrawal K C, et al. Adenovirus-mediated inhibition of NF-kappaB confers chemo-sensitization and apoptosis in prostate cancer cells. Int J Oncol. 2003 August; 23(2):317-323.
52. Kim B Y, Kim K A, Kwon O, Kim S O, Kim M S, Kim B S, et al. NF-kappaB inhibition radiosensitizes Ki-Ras-transformed cells to ionizing radiation. Carcinogenesis. 2005 August; 26(8):1395-1403.
53. Gowrishankar K, Gunatilake D, Gallagher S J, Tiffen J, Rizos H, Hersey P. Inducible but not constitutive expression of P D-L1 in human melanoma cells is dependent on activation of NF-κB. PLoS ONE. 2015 Apr. 6; 10(4):e0123410.
54. Chen H, Li C, Peng X, Zhou Z, Weinstein J N, Cancer Genome Atlas Research Network, et al. A Pan-Cancer Analysis of Enhancer Expression in Nearly 9000 Patient Samples. Cell. 2018 Apr. 5; 173(2):386-399.e12.
55. Nishio H, Yaguchi T, Sugiyama J, Sumimoto H, Umezawa K, Iwata T, et al. Immunosuppression through constitutively activated NF-κB signalling in human ovarian cancer and its reversal by an NF-κB inhibitor. Br J Cancer. 2014 Jun. 10; 110(12):2965-2974.
56. Wang D, DuBois R N. Immunosuppression associated with chronic inflammation in the tumor microenvironment. Carcinogenesis. 2015 October; 36(10):1085-1093.
57. Chen X, Kandasamy K, Srivastava R K. Differential roles of RelA (p65) and c-Rel subunits of nuclear factor kappa B in tumor necrosis factor-related apoptosis-inducing ligand signaling. Cancer Res. 2003 Mar. 1; 63(5):1059-1066.
58. Wang X, Wang J, Zheng H, Xie M, Hopewell E L, Albrecht R A, et al. Differential requirement for the IKKβ/NF-κB signaling module in regulating TLR- versus RLR-induced type 1 IFN expression in dendritic cells. J Immunol. 2014 Sep. 1; 193(5):2538-2545.
59. O'Keeffe M, Grumont R J, Hochrein H, Fuchsberger M, Gugasyan R, Vremec D, et al. Distinct roles for the NF-kappaB1 and c-Rel transcription factors in the differentiation and survival of plasmacytoid and conventional dendritic cells activated by TLR-9 signals. Blood. 2005 Nov. 15; 106(10):3457-3464.
60. Gilmore T D, Gerondakis S. The c-Rel Transcription Factor in Development and Disease. Genes Cancer. 2011 July; 2(7):695-711.
61. Oh H, Ghosh S. NF-κB: roles and regulation in different CD4(+) T-cell subsets. Immunol Rev. 2013 March; 252(1):41-51.
62. Li Q, Van Antwerp D, Mercurio F, Lee K F, Verma I M. Severe liver degeneration in mice lacking the IkappaB kinase 2 gene. Science. 1999 Apr. 9; 284(5412):321-325.
63. Li Z W, Chu W, Hu Y, Delhase M, Deerinck T, Ellisman M, et al. The IKKbeta subunit of IkappaB kinase (IKK) is essential for nuclear factor kappaB activation and prevention of apoptosis. J Exp Med. 1999 Jun. 7; 189(11):1839-1845.
64. Arunkumar N, Liu C, Hang H, Song W. Toll-like receptor agonists induce apoptosis in mouse B-cell lymphoma cells by altering NF-κB activation. Cell Mol Immunol. 2013 July; 10(4):360-372.
65. Liang X, Moseman E A, Farrar M A, Bachanova V, Weisdorf D J, Blazar B R, et al. Toll-like receptor 9 signaling by CpG-B oligodeoxynucleotides induces an apoptotic pathway in human chronic lymphocytic leukemia B cells. Blood. 2010 Jun. 17; 115(24):5041-5052.
66. Ben Abdelwahed R, Cosette J, Donnou S, Crozet L, Ouakrim H, Fridman W H, et al. Lymphoma B-cell responsiveness to CpG-DNA depends on the tumor microenvironment. J Exp Clin Cancer Res. 2013 Apr. 5; 32:18.
67. Sagiv-Barfi I, Kohrt H E, Burckhardt L, Czerwinski D K, Levy R. Ibrutinib enhances the antitumor immune response induced by intratumoral injection of a TLR9 ligand in mouse lymphoma. Blood. 2015 Mar. 26; 125(13):2079-2086.
68. Czerwinski D K, Frank M J, Shree T, Khodadoust M S, Long S R, Levy R. Intratumoral Injection of CpG-ODN Plus Systemic Ibrutinib Induces an Anti-Tumor Immune Response Affecting T Cell Subsets in the Microenvironment of Both Injected and Non-Injected Tumor Sites in Patients with Low-Grade Lymphoma. Blood [Internet]. 2018 Nov. 21; Available from: http://www.bloodjournal.org/content/132/Suppl_1/1612
69. TLR9 Agonist SD-101, Anti-OX40 Antibody BMS 986178, and Radiation Therapy in Treating Patients With Low-Grade B-Cell Non-Hodgkin Lymphomas—Full Text View—ClinicalTrials.gov [Internet]. [cited 2019 May 14]. Available from: https://clinicaltrials.gov/ct2/show/NCT03410901
70. Brody J D, Ai W Z, Czerwinski D K, Torchia J A, Levy M, Advani R H, et al. In situ vaccination with a TLR9 agonist induces systemic lymphoma regression: a phase I/II study. J Clin Oncol. 2010 Oct. 1; 28(28):4324-4332.
71. Houot R, Levy R. T-cell modulation combined with intratumoral CpG cures lymphoma in a mouse model without the need for chemotherapy. Blood. 2009 Apr. 9; 113(15):3546-3552.
72. Wang D, Jiang W, Zhu F, Mao X, Agrawal S. Modulation of the tumor microenvironment by intratumoral administration of IMO-2125, a novel TLR9 agonist, for cancer immunotherapy. Int J Oncol. 2018 September; 53(3):1193-1203.
73. Bai L, Chen W, Chen J, Li W, Zhou L, Niu C, et al. Heterogeneity of Toll-like receptor 9 signaling in B cell malignancies and its potential therapeutic application. J Transl Med. 2017 Feb. 27; 15(1):51.
74. Jahrsdorfer B, Mühlenhoff L, Blackwell S E, Wagner M, Poeck H, Hartmann E, et al. B-cell lymphomas differ in their responsiveness to CpG oligodeoxynucleotides. Clin Cancer Res. 2005 Feb. 15; 11(4):1490-1499.
75. Noack J, Jordi M, Zauner L, Alessi D, Burch A, Tinguely M, et al. TLR9 agonists induced cell death in Burkitt's lymphoma cells is variable and influenced by TLR9 polymorphism. Cell Death Dis. 2012 Jun. 21; 3:e323.
76. Guiducci C, Ghirelli C, Marloie-Provost M-A, Matray T, Coffman R L, Liu Y-J, et al. PI3K is critical for the nuclear translocation of IRF-7 and type I IFN production by human plasmacytoid predendritic cells in response to TLR activation. J Exp Med. 2008 Feb. 18; 205(2):315-322.
77. Nandagopal N, Ali A K, Komal A K, Lee S-H. The Critical Role of IL-15-PI3K-mTOR Pathway in Natural Killer Cell Effector Functions. Front Immunol. 2014 Apr. 23; 5:187.
78. Finotti A, Borgatti M, Bezzerri V, Nicolis E, Lampronti I, Dechecchi M, et al. Effects of decoy molecules targeting NF-kappaB transcription factors in Cystic fibrosis IB3-1 cells: recruitment of NF-kappaB to the IL-8 gene promoter and transcription of the IL-8 gene. Artif DNA PNA XNA. 2012 June; 3(2):97-296.
79. De Stefano D, Coletta C, Bianca R d'Emmanuele di V, Falcone L, d Angelo I, Ungaro F, et al. A decoy oligonucleotide to NF-κB delivered through inhalable particles prevents LPS-induced rat airway inflammation. Am J Respir Cell Mol Biol. 2013 August; 49(2):288-295.
80. Fichtner-Feigl S, Fuss I J, Preiss J C, Strober W, Kitani A. Treatment of murine Th1- and Th2-mediated inflammatory bowel disease with N F-kappa B decoy oligonucleotides. J Clin Invest. 2005 November; 115(11):3057-3071.
81. Fisher L, Soomets U, Cortes Toro V, Chilton L, Jiang Y, Langel U, et al. Cellular delivery of a double-stranded oligonucleotide NFkappaB decoy by hybridization to complementary PNA linked to a cell-penetrating peptide. Gene Ther. 2004 August; 11(16):1264-1272.
82. Wardwell P R, Bader R A. Immunomodulation of cystic fibrosis epithelial cells via NF-κB decoy oligonucleotide-coated polysaccharide nanoparticles. J Biomed Mater Res A. 2015 May; 103(5):1622-1631.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound comprising a first nucleic acid sequence capable of binding to Nuclear Factor Kappa-Light-Chain-Enhancer of Activated B Cells (NF-κB) and a second nucleic acid sequence capable of binding a Toll-like receptor (TLR) protein, wherein said first nucleic acid sequence and said second nucleic acid sequence are covalently bound through a covalent spacer, wherein said covalent spacer is a bond, a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

2. The compound of claim 1, wherein the first nucleic acid sequence capable of binding to NF-κB comprises a first NF-κB binding site nucleic acid sequence and a second NF-κB binding site nucleic acid sequence connected through a first spacer, wherein said first spacer is a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

3. The compound of claim 1, wherein the Toll-like receptor protein is human Toll-like receptor 3, Toll-like receptor 7, Toll-like receptor 8, or Toll-like receptor 9.

4. (canceled)

5. (canceled)

6. The compound of claim 1, wherein the second nucleic acid sequence comprises an unmethylated CpG motif.

7. (canceled)

8. The compound of claim 1, wherein the second nucleic acid sequence capable of binding a TLR protein comprises a first TLR binding site nucleic acid sequence and a second TLR site nucleic acid sequence connected through a second spacer, wherein said second spacer is a substituted or unsubstituted polyglycol, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

9. The compound of claim 1, further comprising a phosphorothioate linkage in the first nucleic acid sequence or the second nucleic acid sequence.

10. The compound of claim 1, further comprising a plurality of phosphorothioate linkages in the first nucleic acid sequence or the second nucleic acid sequence.

11. (canceled)

12. (canceled)

13. The compound of claim 2, wherein the first spacer has the formula: 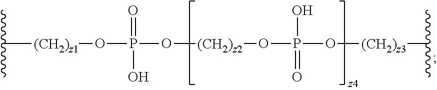
wherein
z1, z2, z3 and z4 are independently integers from 0 to 20.

14. The compound of claim 2, wherein the first spacer is a substituted or unsubstituted C₁-C₄₀ alkylene, substituted or unsubstituted 2 to 40 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

15. The compound of claim 1, wherein the covalent spacer has the formula: 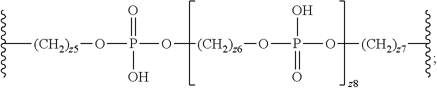
wherein
z5, z6, z7 and z8 are independently integers from 0 to 20.

16. The compound of claim 1, wherein the covalent spacer is a substituted or unsubstituted C₁-C₄₀ alkylene, substituted or unsubstituted 2 to 40 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

17. The compound of claim 1, further comprising a first terminal moiety that is covalently bound through a third spacer to the first nucleic acid sequence.

18. The compound of claim 17, wherein the third spacer has the formula: 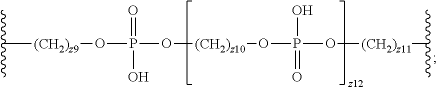
wherein
z9, z10, z11 and z12 are independently integers from 0 to 20.

19. The compound of claim 1, further comprising a second terminal moiety that is covalently bound through a fourth spacer to the second nucleic acid sequence.

20. The compound of claim 19, wherein the fourth spacer has the formula: 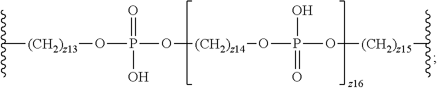
wherein
z13, z14, z15 and z16 are independently integers from 0 to 20.

21. The compound of claim 1, further comprising a first terminal moiety that is covalently bound through a third spacer to the first nucleic acid sequence, and a second terminal moiety that is covalently bound through a fourth spacer to the second nucleic acid sequence.

22. The compound of claim 21, wherein the first terminal moiety and second terminal moiety are independently a hydrogen, monophosphate, polyphosphate, —OH, —NH₂, or

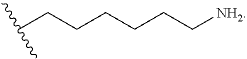 [SMILES: *CCCCCCN]

23. (canceled)

24. The compound of claim 1, comprising the sequence: SEQ ID NO: 5.

25. A pharmaceutical composition comprising a pharmaceutically acceptable excipient and the compound of claim 1.

26. A method of treating a disease selected from cancer, an infectious disease, an autoimmune disease, and an inflammatory disease in a patient in need of such treatment, the method comprising administering a therapeutically effective amount of a compound of claim 1 to a patient in need thereof.

27.-53. (canceled)


SEQUENCE LISTING (21 sequences)

SEQ ID NO: 1 - DNA, 26 bp, Artificial Sequence
  Synthetic polynucleotide
  misc_feature (1)..(1): 5′ terminus modified by 3-(((3-(((3-(((3-(((3-hydroxypropoxy)oxidophosphoryl)oxy)propoxy) oxidophosphoryl)oxy)propoxy)oxidophosphoryl)oxy)propoxy)oxidophos phoryl)oxy)propyl
  misc_feature (13)..(14): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (26)..(26): 3′ terminus modified by 3-hydroxypropyl (3-((oxido(3-((oxido(3-((oxidophosphoryl)oxy)propoxy)phosphoryl) oxy)propoxy)phosphoryl)oxy)propyl)phosphate
tggggactttccatggaaagtcccca

SEQ ID NO: 2 - DNA, 26 bp, Artificial Sequence
  Synthetic polynucleotide
  misc_feature (1)..(1): 5′ terminus modified by 3-(((3-(((3-(((3-(((3-hydroxypropoxy)oxidophosphoryl)oxy)propoxy) oxidophosphoryl)oxy)propoxy)oxidophosphoryl)oxy)propoxy)oxidophos phoryl)oxy)propyl
  misc_feature (13)..(14): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (26)..(26): 3′ terminus modified by 3-hydroxypropyl (3-((oxido(3-((oxido(3-((oxidophosphoryl)oxy)propoxy)phosphoryl) oxy)propoxy)phosphoryl)oxy)propyl)phosphate
tggaaagtccccatggggactttcca

SEQ ID NO: 3 - DNA, 44 bp, Artificial Sequence
  Synthetic polynucleotide
  misc_feature (1)..(1): 5′ terminus modified by 3-(((3-(((3-(((3-(((3-hydroxypropoxy)oxidophosphoryl)oxy)propoxy) oxidophosphoryl)oxy)propoxy)oxidophosphoryl)oxy)propoxy)oxidophos phoryl)oxy)propyl
  misc_feature (1)..(2): phosphorothioate linker
  misc_feature (2)..(3): phosphorothioate linker
  misc_feature (3)..(4): phosphorothioate linker
  misc_feature (22)..(23): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (41)..(42): phosphorothioate linker
  misc_feature (42)..(43): phosphorothioate linker
  misc_feature (43)..(44): phosphorothioate linker
  misc_feature (44)..(44): 3′ terminus modified by 3-hydroxypropyl (3-((oxido(3-((oxido(3-((oxidophosphoryl)oxy)propoxy)phosphoryl)o xy)propoxy)phosphoryl)oxy)propyl) phosphate
gatcgaggggactttccctagcgctagggaaagtcccctcgatc

SEQ ID NO: 4 - DNA, 40 bp, Artificial Sequence
  Synthetic polynucleotide
  misc_feature (1)..(1): 5′ terminus modified by 3-(((3-(((3-(((3-(((3-hydroxypropoxy)oxidophosphoryl)oxy)propoxy) oxidophosphoryl)oxy)propoxy)oxidophosphoryl)oxy)propoxy)oxidophos phoryl)oxy)propyl
  misc_feature (1)..(2): phosphorothioate linker
  misc_feature (2)..(3): phosphorothioate linker
  misc_feature (3)..(4): phosphorothioate linker
  misc_feature (20)..(21): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (37)..(38): phosphorothioate linker
  misc_feature (38)..(39): phosphorothioate linker
  misc_feature (39)..(40): phosphorothioate linker
  misc_feature (40)..(40): 3′ terminus modified by 3-hydroxypropyl (3-((oxido(3-((oxido(3-((oxidophosphoryl)oxy)propoxy)phosphoryl) oxy)propoxy)phosphoryl)oxy)propyl)phosphate
ccttgaagggatttccctccggagggaaatcccttcaagg

SEQ ID NO: 5 - DNA, 60 bp, Artificial Sequence
  Synthetic polynucleotide
  misc_feature (1)..(1): deoxythymidine
  misc_feature (1)..(2): phosphorothioate linker
  misc_feature (2)..(3): phosphorothioate linker
  misc_feature (3)..(4): phosphorothioate linker
  misc_feature (4)..(5): phosphorothioate linker
  misc_feature (5)..(6): phosphorothioate linker
  misc_feature (6)..(7): phosphorothioate linker
  misc_feature (7)..(8): phosphorothioate linker
  misc_feature (8)..(9): phosphorothioate linker
  misc_feature (9)..(10): phosphorothioate linker
  misc_feature (10)..(11): phosphorothioate linker
  misc_feature (11)..(12): phosphorothioate linker
  misc_feature (12)..(13): phosphorothioate linker
  misc_feature (13)..(14): phosphorothioate linker
  misc_feature (14)..(15): phosphorothioate linker
  misc_feature (15)..(16): phosphorothioate linker
  misc_feature (16)..(17): phosphorothioate linker
  misc_feature (17)..(18): phosphorothioate linker
  misc_feature (18)..(19): phosphorothioate linker
  misc_feature (19)..(20): phosphorothioate linker
  misc_feature (20)..(21): 3-(((3-(((3-(((3-(((3-propoxy)oxidophosphoryl) oxy)propoxy)oxidophosphoryl)oxy)propoxy)oxidophosphoryl)oxy) propoxy)oxidophosphoryl)oxy)propyl phosphoryl linker
  misc_feature (21)..(22): phosphorothioate linker
  misc_feature (22)..(23): phosphorothioate linker
  misc_feature (23)..(24): phosphorothioate linker
  misc_feature (40)..(41): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (57)..(58): phosphorothioate linker
  misc_feature (58)..(59): phosphorothioate linker
  misc_feature (59)..(60): phosphorothioate linker
  misc_feature (60)..(60): 3′ terminus modified by 3-hydroxypropyl (3-((oxido(3-((oxido(3-((oxidophosphoryl)oxy)propoxy)phosphoryl) oxy)propoxy)phosphoryl)oxy)propyl)phosphate
tccatgacgttcctgatgctccttgaagggatttccctccggagggaaatcccttcaagg

SEQ ID NO: 6 - DNA, 60 bp, Artificial Sequence
  Synthetic polynucleotide
  misc_feature (1)..(1): deoxythymidine
  misc_feature (1)..(2): phosphorothioate linker
  misc_feature (2)..(3): phosphorothioate linker
  misc_feature (3)..(4): phosphorothioate linker
  misc_feature (4)..(5): phosphorothioate linker
  misc_feature (5)..(6): phosphorothioate linker
  misc_feature (6)..(7): phosphorothioate linker
  misc_feature (7)..(8): phosphorothioate linker
  misc_feature (8)..(9): phosphorothioate linker
  misc_feature (9)..(10): phosphorothioate linker
  misc_feature (10)..(11): phosphorothioate linker
  misc_feature (11)..(12): phosphorothioate linker
  misc_feature (12)..(13): phosphorothioate linker
  misc_feature (13)..(14): phosphorothioate linker
  misc_feature (14)..(15): phosphorothioate linker
  misc_feature (15)..(16): phosphorothioate linker
  misc_feature (16)..(17): phosphorothioate linker
  misc_feature (17)..(18): phosphorothioate linker
  misc_feature (18)..(19): phosphorothioate linker
  misc_feature (19)..(20): phosphorothioate linker
  misc_feature (20)..(21): 3-(((3-(((3-(((3-(((3-propoxy)oxidophosphoryl) oxy)propoxy)oxidophosphoryl)oxy)propoxy)oxidophosphoryl)oxy) propoxy)oxidophosphoryl)oxy)propyl phosphoryl linker
  misc_feature (21)..(22): phosphorothioate linker
  misc_feature (22)..(23): phosphorothioate linker
  misc_feature (23)..(24): phosphorothioate linker
  misc_feature (40)..(41): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (57)..(58): phosphorothioate linker
  misc_feature (58)..(59): phosphorothioate linker
  misc_feature (59)..(60): phosphorothioate linker
  misc_feature (60)..(60): 3′ terminus modified by 3-hydroxypropyl (3-((oxido(3-((oxido(3-((oxidophosphoryl)oxy)propoxy)phosphoryl) oxy)propoxy)phosphoryl)oxy)propyl)phosphate
tccatgagcttcctgatgctccttgaagggatttccctccggagggaaatcccttcaagg

SEQ ID NO: 7 - DNA, 60 bp, Artificial Sequence
  Synthetic polynucleotide
  misc_feature (1)..(1): deoxyguanosine
  misc_feature (1)..(2): phosphorothioate linker
  misc_feature (2)..(3): phosphorothioate linker
  misc_feature (16)..(17): phosphorothioate linker
  misc_feature (17)..(18): phosphorothioate linker
  misc_feature (18)..(19): phosphorothioate linker
  misc_feature (19)..(20): phosphorothioate linker
  misc_feature (20)..(21): 3-(((3-(((3-(((3-(((3-propoxy)oxidophosphoryl) oxy)propoxy)oxidophosphoryl)oxy)propoxy)oxidophosphoryl)oxy) propoxy)oxidophosphoryl)oxy)propyl phosphoryl linker
  misc_feature (21)..(22): phosphorothioate linker
  misc_feature (22)..(23): phosphorothioate linker
  misc_feature (23)..(24): phosphorothioate linker
  misc_feature (40)..(41): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (57)..(58): phosphorothioate linker
  misc_feature (58)..(59): phosphorothioate linker
  misc_feature (59)..(60): phosphorothioate linker
  misc_feature (60)..(60): 3′ terminus modified by 3-hydroxypropyl (3-((oxido(3-((oxido(3-((oxidophosphoryl)oxy)propoxy)phosphoryl) oxy)propoxy)phosphoryl)oxy)propyl)phosphate
ggtgcatcgatgcaggggggccttgaagggatttccctccggagggaaatcccttcaagg

SEQ ID NO: 8 - DNA, 60 bp, Artificial Sequence
  Synthetic polynucleotide
  misc_feature (1)..(1): deoxyguanosine
  misc_feature (1)..(2): phosphorothioate linker
  misc_feature (2)..(3): phosphorothioate linker
  misc_feature (16)..(17): phosphorothioate linker
  misc_feature (17)..(18): phosphorothioate linker
  misc_feature (18)..(19): phosphorothioate linker
  misc_feature (19)..(20): phosphorothioate linker
  misc_feature (20)..(21): 3-(((3-(((3-(((3-(((3-propoxy)oxidophosphoryl) oxy)propoxy)oxidophosphoryl)oxy)propoxy)oxidophosphoryl)oxy) propoxy)oxidophosphoryl)oxy)propyl phosphoryl linker
  misc_feature (21)..(22): phosphorothioate linker
  misc_feature (22)..(23): phosphorothioate linker
  misc_feature (23)..(24): phosphorothioate linker
  misc_feature (40)..(41): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (57)..(58): phosphorothioate linker
  misc_feature (58)..(59): phosphorothioate linker
  misc_feature (59)..(60): phosphorothioate linker
  misc_feature (60)..(60): 3′ terminus modified by 3-hydroxypropyl (3-((oxido(3-((oxido(3-((oxidophosphoryl)oxy)propoxy)phosphoryl) oxy)propoxy)phosphoryl)oxy)propyl)phosphate
ggtgcatgcatgcaggggggccttgaagggatttccctccggagggaaatcccttcaagg

SEQ ID NO: 9 - DNA, 26 bp, Artificial Sequence
  Synthetic polynucleotide
  misc_feature (1)..(1): 5′ terminus modified by 3-(((3-(((3-(((3-(((3-hydroxypropoxy)oxidophosphoryl)oxy)propoxy) oxidophosphoryl)oxy)propoxy)oxidophosphoryl)oxy)propoxy)oxidophos phoryl)oxy)propyl
  misc_feature (13)..(14): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (26)..(26): 3′ terminus modified by 6-aminohexyl (3-((oxido(3-((oxido(3-((oxidophosphoryl)oxy)propoxy)phosphoryl) oxy)propoxy)phosphoryl)oxy)propyl)phosphate
tggggactttccatggaaagtcccca

SEQ ID NO: 10 - DNA, 26 bp, Artificial Sequence
  Synthetic polynucleotide
  misc_feature (1)..(1): 5′ terminus modified by 3-(((3-(((3-(((3-(((3-hydroxypropoxy)oxidophosphoryl)oxy)propoxy) oxidophosphoryl)oxy)propoxy)oxidophosphoryl)oxy)propoxy) oxidophosphoryl)oxy)propyl
  misc_feature (13)..(14): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (26)..(26): 3′ terminus modified by 6-aminohexyl (3-((oxido(3-((oxido(3-((oxidophosphoryl)oxy)propoxy)phosphoryl) oxy)propoxy)phosphoryl)oxy)propyl)phosphate
tggaaagtccccatggggactttcca

SEQ ID NO: 11 - DNA, 44 bp, Artificial Sequence; identical to SEQ ID NO: 3
  Synthetic polynucleotide
  misc_feature (1)..(1): 5′ terminus modified by 3-(((3-(((3-(((3-(((3-hydroxypropoxy)oxidophosphoryl)oxy)propoxy) oxidophosphoryl)oxy)propoxy)oxidophosphoryl)oxy)propoxy) oxidophosphoryl)oxy)propyl
  misc_feature (1)..(2): phosphorothioate linker
  misc_feature (2)..(3): phosphorothioate linker
  misc_feature (3)..(4): phosphorothioate linker
  misc_feature (22)..(23): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (41)..(42): phosphorothioate linker
  misc_feature (42)..(43): phosphorothioate linker
  misc_feature (43)..(44): phosphorothioate linker
  misc_feature (44)..(44): 3′ terminus modified by 6-aminohexyl (3-((oxido(3-((oxido(3-((oxidophosphoryl)oxy)propoxy)phosphoryl) oxy)propoxy)phosphoryl)oxy)propyl) phosphate

SEQ ID NO: 12 - DNA, 40 bp, Artificial Sequence; identical to SEQ ID NO: 4
  Synthetic polynucleotide
  misc_feature (1)..(1): 5′ terminus modified by 3-(((3-(((3-(((3-(((3-hydroxypropoxy)oxidophosphoryl)oxy)propoxy) oxidophosphoryl)oxy)propoxy)oxidophosphoryl)oxy)propoxy) oxidophosphoryl)oxy)propyl
  misc_feature (1)..(2): phosphorothioate linker
  misc_feature (2)..(3): phosphorothioate linker
  misc_feature (3)..(4): phosphorothioate linker
  misc_feature (20)..(21): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (37)..(38): phosphorothioate linker
  misc_feature (38)..(39): phosphorothioate linker
  misc_feature (39)..(40): phosphorothioate linker
  misc_feature (40)..(40): 3′ terminus modified by 6-aminohexyl (3-((oxido(3-((oxido(3-((oxidophosphoryl)oxy)propoxy)phosphoryl) oxy)propoxy)phosphoryl)oxy)propyl) phosphate

SEQ ID NO: 13 - DNA, 60 bp, Artificial Sequence; identical to SEQ ID NO: 5
  Synthetic polynucleotide
  misc_feature (1)..(1): deoxythymidine
  misc_feature (1)..(2): phosphorothioate linker
  misc_feature (2)..(3): phosphorothioate linker
  misc_feature (3)..(4): phosphorothioate linker
  misc_feature (4)..(5): phosphorothioate linker
  misc_feature (5)..(6): phosphorothioate linker
  misc_feature (6)..(7): phosphorothioate linker
  misc_feature (7)..(8): phosphorothioate linker
  misc_feature (8)..(9): phosphorothioate linker
  misc_feature (9)..(10): phosphorothioate linker
  misc_feature (10)..(11): phosphorothioate linker
  misc_feature (11)..(12): phosphorothioate linker
  misc_feature (12)..(13): phosphorothioate linker
  misc_feature (13)..(14): phosphorothioate linker
  misc_feature (14)..(15): phosphorothioate linker
  misc_feature (15)..(16): phosphorothioate linker
  misc_feature (16)..(17): phosphorothioate linker
  misc_feature (17)..(18): phosphorothioate linker
  misc_feature (18)..(19): phosphorothioate linker
  misc_feature (19)..(20): phosphorothioate linker
  misc_feature (20)..(21): 3-(((3-(((3-(((3-(((3-propoxy)oxidophosphoryl) oxy)propoxy)oxidophosphoryl)oxy)propoxy)oxidophosphoryl)oxy) propoxy)oxidophosphoryl)oxy)propyl phosphoryl linker
  misc_feature (21)..(22): phosphorothioate linker
  misc_feature (22)..(23): phosphorothioate linker
  misc_feature (23)..(24): phosphorothioate linker
  misc_feature (40)..(41): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (57)..(58): phosphorothioate linker
  misc_feature (58)..(59): phosphorothioate linker
  misc_feature (59)..(60): phosphorothioate linker
  misc_feature (60)..(60): 3′ terminus modified by 6-aminohexyl (3-((oxido(3-((oxido(3-((oxidophosphoryl)oxy)propoxy)phosphoryl) oxy)propoxy)phosphoryl)oxy)propyl) phosphate

SEQ ID NO: 14 - DNA, 60 bp, Artificial Sequence; identical to SEQ ID NO: 6
  Synthetic polynucleotide
  misc_feature (1)..(1): deoxythymidine
  misc_feature (1)..(2): phosphorothioate linker
  misc_feature (2)..(3): phosphorothioate linker
  misc_feature (3)..(4): phosphorothioate linker
  misc_feature (4)..(5): phosphorothioate linker
  misc_feature (5)..(6): phosphorothioate linker
  misc_feature (6)..(7): phosphorothioate linker
  misc_feature (7)..(8): phosphorothioate linker
  misc_feature (8)..(9): phosphorothioate linker
  misc_feature (9)..(10): phosphorothioate linker
  misc_feature (10)..(11): phosphorothioate linker
  misc_feature (11)..(12): phosphorothioate linker
  misc_feature (12)..(13): phosphorothioate linker
  misc_feature (13)..(14): phosphorothioate linker
  misc_feature (14)..(15): phosphorothioate linker
  misc_feature (15)..(16): phosphorothioate linker
  misc_feature (16)..(17): phosphorothioate linker
  misc_feature (17)..(18): phosphorothioate linker
  misc_feature (18)..(19): phosphorothioate linker
  misc_feature (19)..(20): phosphorothioate linker
  misc_feature (20)..(21): 3-(((3-(((3-(((3-(((3-propoxy)oxidophosphoryl) oxy)propoxy)oxidophosphoryl)oxy)propoxy)oxidophosphoryl)oxy) propoxy)oxidophosphoryl)oxy)propyl phosphoryl linker
  misc_feature (21)..(22): phosphorothioate linker
  misc_feature (22)..(23): phosphorothioate linker
  misc_feature (23)..(24): phosphorothioate linker
  misc_feature (40)..(41): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (57)..(58): phosphorothioate linker
  misc_feature (58)..(59): phosphorothioate linker
  misc_feature (59)..(60): phosphorothioate linker
  misc_feature (60)..(60): 3′ terminus modified by 6-aminohexyl (3-((oxido(3-((oxido(3-((oxidophosphoryl)oxy)propoxy)phosphoryl) oxy)propoxy)phosphoryl)oxy)propyl) phosphate

SEQ ID NO: 15 - DNA, 60 bp, Artificial Sequence; identical to SEQ ID NO: 7
  Synthetic polynucleotide
  misc_feature (1)..(1): deoxyguanosine
  misc_feature (1)..(2): phosphorothioate linker
  misc_feature (2)..(3): phosphorothioate linker
  misc_feature (16)..(17): phosphorothioate linker
  misc_feature (17)..(18): phosphorothioate linker
  misc_feature (18)..(19): phosphorothioate linker
  misc_feature (19)..(20): phosphorothioate linker
  misc_feature (20)..(21): 3-(((3-(((3-(((3-(((3-propoxy)oxidophosphoryl) oxy)propoxy)oxidophosphoryl)oxy)propoxy)oxidophosphoryl)oxy) propoxy)oxidophosphoryl)oxy)propyl phosphoryl linker
  misc_feature (21)..(22): phosphorothioate linker
  misc_feature (22)..(23): phosphorothioate linker
  misc_feature (23)..(24): phosphorothioate linker
  misc_feature (40)..(41): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (57)..(58): phosphorothioate linker
  misc_feature (58)..(59): phosphorothioate linker
  misc_feature (59)..(60): phosphorothioate linker
  misc_feature (60)..(60): 3′ terminus modified by 6-aminohexyl (3-((oxido(3-((oxido(3-((oxidophosphoryl)oxy)propoxy)phosphoryl) oxy)propoxy)phosphoryl)oxy)propyl) phosphate

SEQ ID NO: 16 - DNA, 60 bp, Artificial Sequence; identical to SEQ ID NO: 8
  Synthetic polynucleotide
  misc_feature (1)..(1): deoxyguanosine
  misc_feature (1)..(2): phosphorothioate linker
  misc_feature (2)..(3): phosphorothioate linker
  misc_feature (16)..(17): phosphorothioate linker
  misc_feature (17)..(18): phosphorothioate linker
  misc_feature (18)..(19): phosphorothioate linker
  misc_feature (19)..(20): phosphorothioate linker
  misc_feature (20)..(21): 3-(((3-(((3-(((3-(((3-propoxy)oxidophosphoryl) oxy)propoxy)oxidophosphoryl)oxy)propoxy)oxidophosphoryl)oxy) propoxy)oxidophosphoryl)oxy)propyl phosphoryl linker
  misc_feature (21)..(22): phosphorothioate linker
  misc_feature (22)..(23): phosphorothioate linker
  misc_feature (23)..(24): phosphorothioate linker
  misc_feature (40)..(41): 3-(((3-(((3-(((3-propoxy)oxidophosphoryl) oxy)propoxy)oxidophosphoryl)oxy)propoxy)oxidophosphoryl)oxy) propyl phosphoryl linker
  misc_feature (57)..(58): phosphorothioate linker
  misc_feature (58)..(59): phosphorothioate linker
  misc_feature (59)..(60): phosphorothioate linker
  misc_feature (60)..(60): 3′ terminus modified by 6-aminohexyl (3-((oxido(3-((oxido(3-((oxidophosphoryl)oxy)propoxy)phosphoryl) oxy)propoxy)phosphoryl)oxy)propyl) phosphate

SEQ ID NO: 17 - DNA, 50 bp, Artificial Sequence
  Synthetic polynucleotide
  misc_feature (1)..(1): deoxythymidine
  misc_feature (1)..(2): phosphorothioate linker
  misc_feature (2)..(3): phosphorothioate linker
  misc_feature (3)..(4): phosphorothioate linker
  misc_feature (4)..(5): phosphorothioate linker
  misc_feature (5)..(6): phosphorothioate linker
  misc_feature (6)..(7): phosphorothioate linker
  misc_feature (7)..(8): phosphorothioate linker
  misc_feature (8)..(9): phosphorothioate linker
  misc_feature (9)..(10): phosphorothioate linker
  misc_feature (10)..(11): phosphorothioate linker
  misc_feature (11)..(12): phosphorothioate linker
  misc_feature (12)..(13): phosphorothioate linker
  misc_feature (13)..(14): phosphorothioate linker
  misc_feature (14)..(15): phosphorothioate linker
  misc_feature (15)..(16): phosphorothioate linker
  misc_feature (16)..(17): phosphorothioate linker
  misc_feature (17)..(18): phosphorothioate linker
  misc_feature (18)..(19): phosphorothioate linker
  misc_feature (19)..(20): phosphorothioate linker
  misc_feature (20)..(21): 3-(((3-(((3-(((3-(((3-propoxy)oxidophosphoryl) oxy)propoxy)oxidophosphoryl)oxy)propoxy)oxidophosphoryl)oxy) propoxy)oxidophosphoryl)oxy)propyl phosphoryl linker
  misc_feature (21)..(22): phosphorothioate linker
  misc_feature (22)..(23): phosphorothioate linker
  misc_feature (23)..(24): phosphorothioate linker
  misc_feature (35)..(36): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (48)..(49): phosphorothioate linker
  misc_feature (49)..(50): phosphorothioate linker
  misc_feature (50)..(50): phosphorothioate linker
  misc_feature (50)..(50): 3′ terminus modified by 3-hydroxypropyl (3-((oxido(3-((oxido(3-((oxidophosphoryl)oxy)propoxy)phosphoryl) oxy)propoxy)phosphoryl)oxy)propyl) phosphate
tccatgacgttcctgatgctactcttgccaattacgtaattggcaagagt

SEQ ID NO: 18 - protein, 288 aa, Artificial Sequence
  Synthetic polypeptide
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 19 - protein, 290 aa, Artificial Sequence
  Synthetic polypeptide
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 20 - protein, 223 aa, Artificial Sequence
  Synthetic polypeptide
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 21 - DNA, 50 bp, Artificial Sequence; identical to SEQ ID NO: 17
  Synthetic polynucleotide
  misc_feature (1)..(1): deoxythymidine
  misc_feature (1)..(2): phosphorothioate linker
  misc_feature (2)..(3): phosphorothioate linker
  misc_feature (3)..(4): phosphorothioate linker
  misc_feature (4)..(5): phosphorothioate linker
  misc_feature (5)..(6): phosphorothioate linker
  misc_feature (6)..(7): phosphorothioate linker
  misc_feature (7)..(8): phosphorothioate linker
  misc_feature (8)..(9): phosphorothioate linker
  misc_feature (9)..(10): phosphorothioate linker
  misc_feature (10)..(11): phosphorothioate linker
  misc_feature (11)..(12): phosphorothioate linker
  misc_feature (12)..(13): phosphorothioate linker
  misc_feature (13)..(14): phosphorothioate linker
  misc_feature (14)..(15): phosphorothioate linker
  misc_feature (15)..(16): phosphorothioate linker
  misc_feature (16)..(17): phosphorothioate linker
  misc_feature (17)..(18): phosphorothioate linker
  misc_feature (18)..(19): phosphorothioate linker
  misc_feature (19)..(20): phosphorothioate linker
  misc_feature (20)..(21): 3-(((3-(((3-(((3-(((3-propoxy)oxidophosphoryl) oxy)propoxy)oxidophosphoryl)oxy)propoxy)oxidophosphoryl)oxy) propoxy)oxidophosphoryl)oxy)propyl phosphoryl linker
  misc_feature (21)..(22): phosphorothioate linker
  misc_feature (22)..(23): phosphorothioate linker
  misc_feature (23)..(24): phosphorothioate linker
  misc_feature (35)..(36): 3-(((3-(((3-(((3-propoxy)oxidophosphoryl)oxy) propoxy)oxidophosphoryl)oxy)propoxy)oxidophosphoryl)oxy)propyl phosphoryl linker
  misc_feature (48)..(49): phosphorothioate linker
  misc_feature (49)..(50): phosphorothioate linker
  misc_feature (50)..(50): phosphorothioate linker
  misc_feature (50)..(50): 3′ terminus modified by 6-aminohexyl (3-((oxido(3-((oxido(3-((oxidophosphoryl)oxy)propoxy)phosphoryl) oxy)propoxy)phosphoryl)oxy)propyl) phosphate